(19) United States
(12) Patent Application Publication
(10) Pub. No.: US 2026/0115107 A1
(43) Pub. Date: Apr. 30, 2026

(54) FUNCTIONALIZED ANTI-MICROBIAL PARTICLES

(71) Applicant: NOBIO LTD., Kadima (IL)

(72) Inventors: Nathan ZALTSMAN, Hadera (IL); Yuval SOLLENBERGER, Kadima (IL)

(73) Assignee: NOBIO LTD., Kadima (IL)

(21) Appl. No.: 18/835,772

(22) PCT Filed: Feb. 12, 2023
(86) PCT No.: PCT/IL2023/050146

§ 371 Date: Aug. 5, 2024

Related U.S. Application Data
(60) Provisional application No. 63/309,583, filed on Feb. 13, 2022.

(51) Int. Cl. A61K 6/896; A61K 6/54; A61K 6/69

(52) U.S. Cl. CPC A61K 6/896; A61K 6/54; A61K 6/69

(57) ABSTRACT

Provided herein an anti-microbial particle comprising organic or inorganic core, at least one anti-microbial active unit and at least one polymerizable unit, wherein both the at least one anti-microbial active unit and the at least one polymerizable unit are covalently attached to the core; uses and compositions thereof.

### FIELD OF THE INVENTION

Provided herein are anti-microbial particles comprising organic or inorganic core, at least one anti-microbial active unit and at least one polymerizable unit, wherein both the at least one anti-microbial active unit and the at least one polymerizable unit are covalently attached to the core; uses and compositions thereof. Matrices for various uses that incorporate the anti-microbial particles are also provided herein.

### BACKGROUND OF THE INVENTION

The overwhelming diversity of bacteria in one individual's skin, gastro-intestinal tract and oral cavity is well documented, demonstrating a complex ecosystem anatomically and dynamically in which poly-microbial biofilms are the norm.

Biofilms formed on tissues outside and inside the organism are the major cause of infectious diseases. For example in the oral cavity, biofilm formed on dental hard or soft tissue are the major cause of caries and periodontal disease (Sbordone L., Bortolaia C., Clin Oral Invesig 2003; 7:181-8). Bacterial biofilm forms on both natural and artificial surfaces.

Special attention is paid in recent years to artificial surfaces contacting organisms, as these surfaces lack the epithelial shedding, a major natural mechanism to combat biofilms, thus biofilm accumulation is becoming a major source of medical problems that may result in life threatening complications. Two major factors influence the susceptibility of a surface to accumulate bacteria: surface roughness and the surface-free energy which is a property of the material used. Surface roughness has a higher influence on the adhesion of bacteria than surface-free energy. In this context, artificial restorative materials typically have a higher surface roughness than natural surfaces, and therefore are more prone to bacterial accumulation. Therefore, the development of new materials that diminishes biofilm formation is a critical topic.

The ultimate goal of the development of materials with antibiofilm properties is to improve health and reduce disease occurrence. None of the existing medical devices can guarantee immediate and comprehensive elimination of biofilm or prevention of secondary infection.

For example, in order to sustain the oral defense, dental materials with the following antibiofilm properties are sought after: (1) inhibition of initial binding of microorganisms (2) preventing biofilm growth, (3) affecting microbial metabolism in the biofilm, (4) killing biofilm bacteria, and (5) detaching biofilm (Busscher H J, Rinastiti M. Siswomihardjo W, van der Mei H C., J Dent Res, 2010; 89:657-65; Marsh P D. J Dent, 2010; 38).

Quaternary ammonium compounds (QACs) are widely used in disinfection of water, surfaces and instruments as well as in textile, leather and food industries because of their relatively low toxicity, broad antimicrobial spectrum, non-volatility and chemical stability. Anti-microbial particles based on quaternary ammonium groups have been developed as disclosed in U.S. Pat. Nos. 11,134,676 and 11,178,867. However, resin-based composites comprising such anti-microbial particles, resulted in leaching from the resin-based composite, and thereby led to decay of anti-microbial activity and increase risk of higher toxicity.

This invention provides anti-microbial particles and resin-based composites comprising the same, with reduced leaching properties and still keeping the anti-microbial activity.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides an anti-microbial particle, comprising a polyhedral oligomeric silsesquioxane (POSS) core wherein each silicon atom of the POSS is functionalized by at least one anti-microbial group or by at least one functional polymerizable group wherein the functional polymerizable group comprises an acrylate (e.g., a substituted or non-substituted acrylate group), an epoxy (e.g., a substituted or non-substituted epoxy group), an isocyanate and/or a vinyl group (e.g., a substituted or non-substituted vinyl group); and wherein the molar ratio of the silicon atom functionalized by at least one anti-microbial group and the silicon atom functionalized by at least one functional polymerizable group is between 10:1 to 1:10, respectively.

One aspect of the present invention provides an anti-microbial particle comprising a polyhedral oligomeric silsesquioxane (POSS) core wherein the silicon atoms of the POSS are functionalized by at least one anti-microbial group and by at least one functional polymerizable group,

wherein the at least one functional polymerizable group comprises a substituted and/or non-substituted acrylate group, epoxy group, vinyl group and/or isocyanate group; and

wherein the anti-microbial active unit is represented by structure (1):

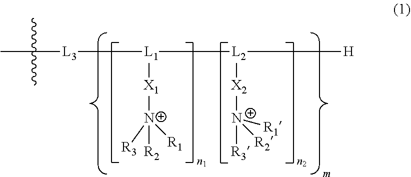

wherein
L₁ is a first linker or a bond;
L₂ is a second linker;
L₃ is a third linker or a bond;
R₁ and R₁′ are each independently alkyl, terpenoid, cycloalkyl, aryl, heterocycle, alkenyl, alkynyl or any combination thereof;
R₂ and R₂′ are each independently alkyl, terpenoid, cycloalkyl, aryl, heterocycle, alkenyl, alkynyl or any combination thereof;
R₃ and R₃′ are each independently nothing, hydrogen, alkyl, terpenoid moiety, cycloalkyl, aryl, heterocycle, a conjugated alkyl, alkenyl, alkynyl or any combination thereof; wherein if R₃ or
R₃′ are nothing, the nitrogen is not charged;
X₁ and X₂ is each independently a bond, alkylene, alkenylene, or alkynylene;
n₁ is each independently an integer between 0 to 200;
n₂ is each independently an integer between 0 to 200;
wherein n₁+n₂≥1;
m is an integer between 1 to 200 and the repeating unit is the same or different; and
the notion [symbol] denotes a covalent bond to an organic or inorganic core.
   In another embodiment, the anti-microbial group is —N(R₁)(R₂)(R₃)⁺, —N(R₁)(R₂)⁺—, —N(R₁′)(R₂′)(R₃′)⁺ or —N(R₁′)(R₂′)⁺— (all possibilities are connected covalently to X₁ or X₂).

One aspect of the present invention provides composition comprising a plurality of disclosed anti-microbial particles and a polymeric material as a matrix.

These, additional, and/or other aspects and/or advantages of the present invention are set forth in the detailed description which follows; possibly inferable from the detailed description; and/or learnable by practice of the present invention.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1C depict a scheme of the anti-microbial particle of this invention, which comprises anti-microbial active unit(s) and polymerizable unit(s), according to some embodiments of the invention. FIG. 1A: a particle having an identical number of anti-microbial active units and polymerizable units (6); FIG. 1B: a particle having more anti-microbial active units compared to polymerizable units (7 compared to 5, respectively); and FIG. 1C: a particle having more polymerizable units compared to anti-microbial active units (7 compared to 5, respectively). In FIGS. 1A-1C, it can be seen that the (anti-microbial active and polymerizable) units can be identical or different as can be inferred from the terms “ . . . unit 1” or “ . . . unit 2”.

FIGS. 2A-2B depict anti-microbial active unit and (anti-microbial) monomeric unit), according to some embodiments of the invention. FIG. 2A: anti-microbial active unit; and FIG. 2B: an (anti-microbial) monomeric unit; wherein the notion “ [symbol] ” is defined as a bond to the inorganic or organic core of the anti-microbial particle of this invention.

FIGS. 3A-3B depict polymerizable unit and (polymerizable) monomeric unit), according to some embodiments of the invention. FIG. 3A: polymerizable unit; and FIG. 3B: a (polymerizable) monomeric unit; wherein the notion “ [symbol] ” is defined as a bond to the inorganic or organic core of the anti-microbial particle of this invention.

FIG. 4 depicts a representative scheme for the preparation of standard particles), according to some embodiments of the invention, wherein the anti-microbial active group is a tertiary amine or a quaternary ammonium group comprising at least one terpenoid moiety and the anti-microbial unit has one monomeric unit (a monomeric backbone); the circles represent the organic or inorganic core; and R¹—Y—R¹ is a C₁-C₄ alkyl and Y is a leaving group such as halogen or sulfonate.

FIG. 5 depicts a representative scheme for the preparation of a standard particle, according to some embodiments of the invention, with the particle having cinnamyl groups with a core (represented by a circle) via amino-functional linker wherein the anti-microbial unit has one monomeric unit (a monomeric backbone). Conversion of the tertiary amine to the quaternary ammonium group is optional, and involves reaction of the tertiary amine with a group R¹—Y wherein R¹ is a C₁-C₄ alkyl and Y is a leaving group such as halogen or sulfonate.

FIGS. 6A-6C depicts a representative scheme of three pathways for the preparation of quaternary ammonium salts (QAS) functionalized standard particle), according to some embodiments of the invention, wherein the anti-microbial unit has one monomeric unit (a monomeric backbone); the circles represents organic or inorganic core. FIG. 6A) by reductive amination to achieve tertiary amine, followed by an alkylation reaction; FIG. 6B) by stepwise alkylation reactions; and FIG. 6C): by reacting a linker functionalized with a leaving group (e.g., Cl or another halogen) with tertiary amine. R¹ and R² represent C₁-C₄ alkyls such as methyl, ethyl, propyl or isopropyl. R¹ and R² may be different or the same group. Y represents any leaving group, for example Cl, Br or I, or a sulfonate (e.g., mesyl, tosyl).

FIGS. 7A-7C depicts a representative scheme of three pathways for the preparation of quaternary ammonium salts (QAS) of functionalized particles with enhanced thermal stability wherein the anti-microbial unit has one monomeric unit (a monomeric backbone), according to some embodiments of the invention. The circles represent organic or inorganic core. FIG. 7A) by alkylation with R₁—Y/R₂—Y to achieve tertiary amine, followed by an benzylation reaction; FIG. 7B) by a similar pathway as in A), done in the reversed order; and FIG. 7C): by reacting a linker functionalized with a leaving group (e.g., Cl or other halogen) with tertiary amine. R₄ and R₅ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —CH₂C(═O)R, —CH₂C(═S)R, —CH₂CF₃, —CH₂NO₂, 1-alkenyl, 1-alkynyl, 2-alkenyl, 2-alkynyl or any combination thereof; where R is alkyl, aryl, cycloalkyl, heterocycle or any combination thereof. Y represents any leaving group, for example Cl, Br or I, or a sulfonate (e.g., mesyl, tosyl).

FIG. 8 depicts schemes of solid support and solution methods for the preparation of standard particles), according to some embodiments of the invention, wherein the anti-microbial unit has one monomeric unit (a monomeric backbone). The circles represent an organic or inorganic core. Q¹, Q² and Q³ are independently selected from the group consisting of ethoxy, methoxy, methyl, ethyl, hydrogen, sulfonate and halide, wherein at least one of Q¹, Q² and Q³ is a leaving group selected from ethoxy, methoxy, sulfonate (e.g., mesyl, tosyl) and halide. For the sake of clarity the scheme presents a case where Q¹, Q² and Q³ represent leaving groups; Q⁴ represents an anti-microbial group; W is selected from the group consisting of NH₂, halide, sulfonate and hydroxyl; and n is an integer between 1 and 16.

FIG. 9 depicts a representative scheme for the preparation of standard particles), according to some embodiments of the invention, the particles having di-cinnamyl groups with core particle (represented as a circle) functionalized utilizing a (EtO)₃Si-C H₂—(CH₂)ₙ—CH₂NH₂ linker by both solid support method and solution method, wherein the anti-microbial unit has one monomeric unit (a monomeric backbone). n is an integer of 1 to 16.

FIG. 10 depicts a representative scheme for the preparation of standard particles by a solid support method, according to some embodiments of the invention, wherein the anti-microbial unit has an oligomeric or polymeric backbone (more than one monomeric unit). The circles represent a core. The starting material is a core terminated on the surface with hydroxyl groups; Q¹⁰¹, Q¹⁰² and Q¹⁰³ and independently alkoxy, alkyl or aryl; LG is Cl, Br, I, mesylate, tosylate or triflate; Hal is Cl, Br or I; q, q¹, q² and q³ are independently an integer between 0-16; R¹ and R² are each independently alkyl, terpenoid, cycloalkyl, aryl, heterocycle, a conjugated alkyl, alkenyl or any combination thereof, and R³ is nothing, hydrogen, alkyl, terpenoid moiety, cycloalkyl, aryl, heterocycle, alkenyl, alkynyl or any combination thereof.

FIG. 11 depicts a representative scheme for the preparation of particles with enhanced thermal stability by a solid support method, according to some embodiments of the invention, wherein the anti-microbial unit has an oligomeric or polymeric backbone (more than one monomeric unit). The circles represent a core. The starting material is a core terminated on the surface with hydroxyl groups; Q¹⁰¹, Q¹⁰² and Q¹⁰³ are each independently alkoxy, alkyl or aryl; LG is Cl, Br, I, mesylate, tosylate or triflate; Hal is Cl, Br or I; q, q¹, q² and q³ are each independently an integer between 0-16; R₄ and R₅ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, and R₆ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof.

FIGS. 12A-12C depict self-polymerization of trialkoxysilane linker of a standard particle), according to some embodiments of the invention. FIG. 12A: self-polymerization of trialkoxysilane linker via solid support method; FIG. 12B: self-polymerization of trialkoxysilane linker in solution; and FIG. 12C: comparison of polymerization of the silane groups versus simple silanization.

FIGS. 13A-13C depict self-polymerization of trialkoxysilane linker of a particle with enhanced thermal stability, according to some embodiments of the invention. FIG. 13A: self-polymerization of trialkoxysilane linker via solid support method; FIG. 13B: self-polymerization of trialkoxysilane linker in solution; and FIG. 13C: comparison of polymerization of the silane groups versus simple silanization.

FIG. 14 depicts a representative scheme for the preparation of standard particles in a solution method, according to some embodiments of the invention, wherein the anti-microbial unit has more than one monomeric unit (e.g., has an oligomeric or polymeric backbone). The circles represent a core. The starting material is a core terminated on the surface with hydroxyl groups; Q¹⁰¹, Q¹⁰² and Q¹⁰³ and independently alkoxy, alkyl or aryl; LG is Cl, Br, I, mesylate, tosylate or triflate; Hal is Cl, Br or I; q, q¹, q² and q³ are independently an integer between 0-16; R¹ and R² are each independently alkyl, terpenoid, cycloalkyl, aryl, heterocycle, a conjugated alkyl, alkenyl or any combination thereof, and R³ is nothing, hydrogen, alkyl, terpenoid moiety, cycloalkyl, aryl, heterocycle, alkenyl, alkynyl or any combination thereof.

FIG. 15 depicts a representative scheme for the preparation of particles with enhanced thermal stability in a solution method, wherein the anti-microbial unit has more than one monomeric unit (e.g., has an oligomeric or polymeric backbone). The circles represent a core. The starting material is a core terminated on the surface with hydroxyl groups; Q¹⁰¹, Q¹⁰² and Q¹⁰³ and independently alkoxy, alkyl or aryl; LG is Cl, Br, I, mesylate, tosylate or triflate; Hal is Cl, Br or I; q, q¹, q² and q³ are independently an integer between 0-16; R₄ and R₅ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof; and R₆ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof.

FIG. 16 depicts a scheme for the preparation of silica based anti-microbial standard particles comprising dimethylethylammonium as the anti-microbial active group, in a solid support method, according to some embodiments of the invention, wherein the anti-microbial unit has more than one monomeric unit (e.g., has an oligomeric or polymeric backbone).

FIG. 17 depicts a scheme for the preparation of silica based anti-microbial standard particles comprising dimethylethylammonium as the anti-microbial active group, in a solution method, according to some embodiments of the invention, wherein the anti-microbial unit has more than one monomeric unit (e.g., has an oligomeric or polymeric backbone).

FIG. 18 depicts a scheme for the preparation of silica based anti-microbial particles with enhanced thermal stability comprising dimethylbenzylammonium as the anti-microbial active group, in a solid support method, according to some embodiments of the invention, wherein the anti-microbial unit has more than one monomeric unit (e.g., has an oligomeric or polymeric backbone).

FIG. 19 depicts a scheme for the preparation of silica based anti-microbial particles with enhanced thermal stability comprising dimethylbenzylammonium as the anti-microbial active group, in a solution method, according to some embodiments of the invention, wherein the anti-microbial unit has more than one monomeric unit (e.g., has an oligomeric or polymeric backbone).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that this invention may be practiced without these specific details. In other instances, well-known methods, procedures, and components have not been described in detail so as not to obscure this invention.

### Anti-Microbial Particles

In some embodiments, provided herein is an anti-microbial particle comprising organic or inorganic core, at least one anti-microbial active unit and at least one polymerizable unit, wherein both the at least one anti-microbial active unit and the at least one polymerizable unit are covalently attached to the core. In one embodiment, the anti-microbial particle comprises a plurality of identical or different anti-microbial active units. In one embodiment, the anti-microbial particle comprises a plurality of identical or different polymerizable units. In one further embodiment, the anti-microbial particle comprises a molar ratio of between 10:1 to 1:10 of identical or different anti-microbial active units to identical or different polymerizable units. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. In another embodiment, each anti-microbial active unit comprises one or more anti-microbial groups, and said anti-microbial active unit is bound chemically (covalently) to the core (surface) directly or indirectly (e.g., via a linker and/or other chemical moieties). In one other embodiment, each polymerizable unit comprises one or more functional polymerizable groups and said polymerizable unit is bound chemically (covalently) to the core (surface) directly or indirectly (e.g., via a linker and/or other chemical moieties). In one specific embodiment, the anti-microbial particle comprises an inorganic core, wherein each silicon or metal atom within the surface of the inorganic core is functionalized by at least one anti-microbial group or by at least one functional polymerizable group, wherein the molar ratio of the silicon or metal atom within the surface of the inorganic core, functionalized by at least one anti-microbial group; and the silicon or metal atom within the surface of the inorganic core, functionalized by at least one functional polymerizable group—is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. In one embodiment, the functional polymerizable group comprises an acrylate, an epoxy, a vinyl or an isocyanate group. In one embodiment, the anti-microbial group is a quaternary ammonium, a tertiary amine, or a tertiary ammonium.

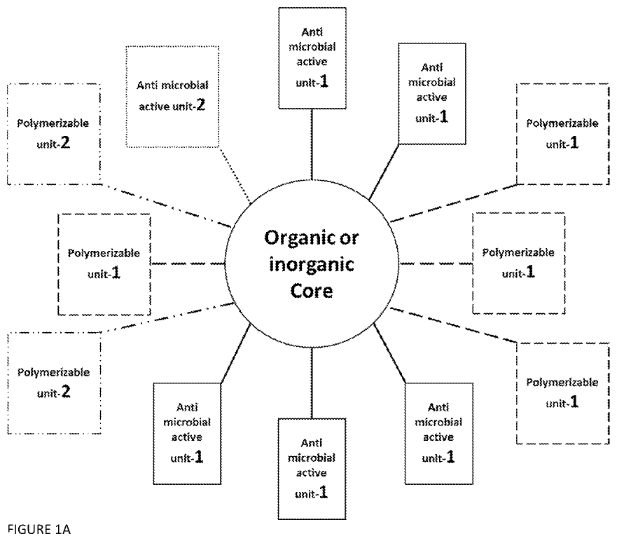

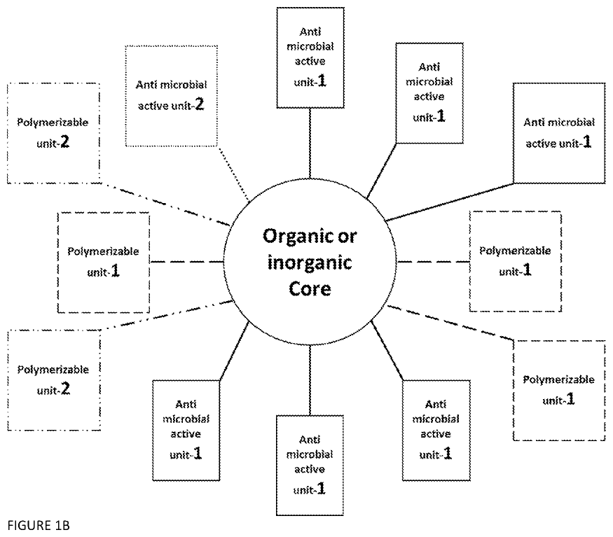

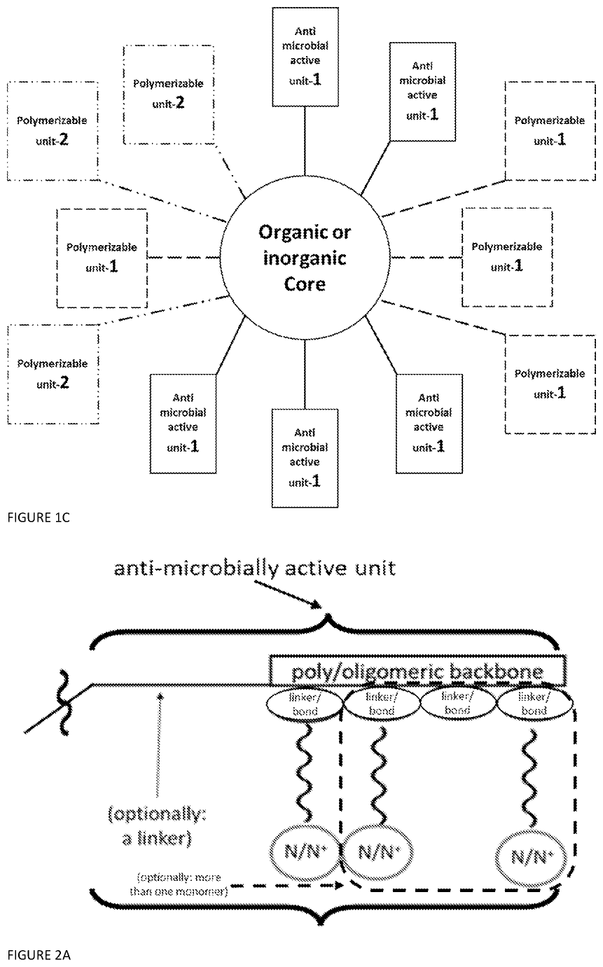

In some non-limiting embodiments, the anti-microbial particles are schematically represented by FIGS. 1A-1C. In FIGS. 1A-1C, it can be seen that the (anti-microbial active and polymerizable) units can be identical or different as can be inferred from the terms “ . . . unit 1” or “ . . . unit 2”. In FIG. 1A the number of anti-microbial active and polymerizable units is identical (6). In FIG. 1B, there are more anti-microbial active units compared to polymerizable units (7 compared to 5, respectively). In FIG. 1C, there are more polymerizable units compared to anti-microbial active units (7 compared to 5, respectively).

In some embodiments, the anti-microbial active unit is schematically represented by FIG. 2A. In one embodiment, the anti-microbial active unit comprises a (polymeric/oligomeric) backbone connected covalently to one or more monomers (or “monomeric units”), wherein said backbone is connected covalently via a bond or a linker to the inorganic or organic core of the anti-microbial particle of this invention (via the notion “ [symbol] ”). In one further embodiment, the monomer or monomeric unit is represented schematically by FIG. 2B. In other embodiment, each monomer or monomeric unit comprises an anti-microbial group.

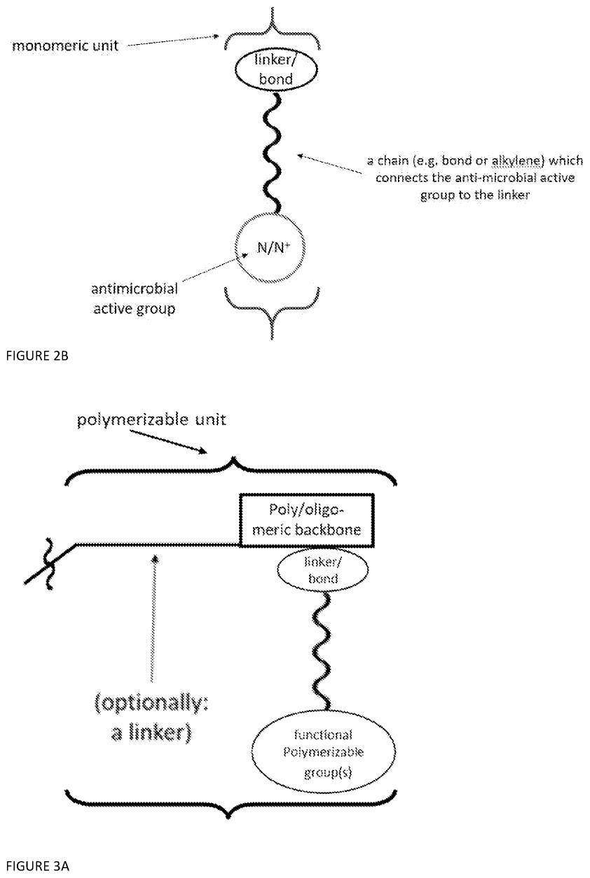

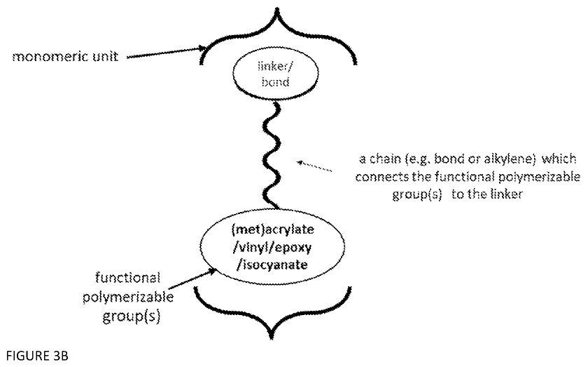

In some embodiments, the polymerizable unit is schematically represented by FIG. 3A. In one embodiment, the polymerizable unit comprises a (polymeric/oligomeric) backbone connected covalently to a monomer (or “monomeric unit”), wherein said backbone is connected covalently via a bond or a linker to the inorganic or organic core of the anti-microbial particle of this invention (via the notion “ [symbol] ”). In one further embodiment, the monomer or monomeric unit is represented schematically by FIG. 3B. In other embodiment, each monomer or monomeric unit comprises at least one functional polymerizable group.

In some embodiments, the number of anti-microbial active units is of between 0.01-30 per one sq nm (nm²) of the core surface of the particle. In another embodiment, the number is between 0.01-20 anti-microbial active units per 1 sq nm of the core surface. In another embodiment, the number is between 0.01-10 anti-microbial active units per 1 sq nm of the core surface. In another embodiment, the number is between 0.01-15 anti-microbial active units per 1 sq nm of the core surface. In another embodiment, the number is between 0.01-5 anti-microbial active units per 1 sq nm of the core surface. Each possibility represents a separate embodiment of this invention.

In some embodiments, the number of polymerizable units is of between 0.01-30 per one sq nm (nm²) of the core surface of the particle. In another embodiment, the number is between 0.01-20 polymerizable units per 1 sq nm of the core surface. In another embodiment, the number is between 0.01-10 polymerizable units per 1 sq nm of the core surface. In another embodiment, the number is between 0.01-15 polymerizable units per 1 sq nm of the core surface. In another embodiment, the number is between 0.01-5 polymerizable units per 1 sq nm of the core surface. Each possibility represents a separate embodiment of this invention.

### Anti-Microbial Active Units

### A) Branched and Non-Branched Units

In some embodiment, the anti-microbial active unit is represented by structure (1):

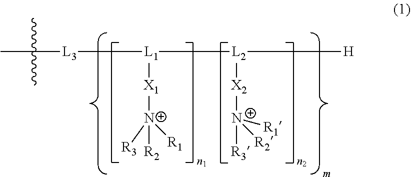

wherein
L₁ is a first linker or a bond;
L₂ is a second linker;
L₃ is a third linker or a bond;
R₁ and R₁′ are each independently alkyl, terpenoid, cycloalkyl, aryl, heterocycle, alkenyl, alkynyl or any combination thereof;
R₂ and R₂′ are each independently alkyl, terpenoid, cycloalkyl, aryl, heterocycle, alkenyl, alkynyl or any combination thereof;
R₃ and R₃′ are each independently nothing, hydrogen, alkyl, terpenoid moiety, cycloalkyl, aryl, heterocycle, a conjugated alkyl, alkenyl, alkynyl or any combination thereof; wherein if R₃ or
R₃′ are nothing, the nitrogen is not charged;
X₁ and X₂ is each independently a bond, alkylene, alkenylene, or alkynylene;
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.
   In another embodiment, the anti-microbial group is —N(R₁)(R₂)(R₃)⁺, —N(R₁)(R₂)⁺—, —N(R₁′)(R₂′)(R₃′)⁺ or —N(R₁′)(R₂′)⁺— (all possibilities are connected covalently to X₁ or X₂).

In another embodiment, the number of the anti-microbial groups per each anti-microbial active unit is at least two, e.g., n₁+n₂≥2 and m≥1 (“branched units”). In another embodiment, the number of the anti-microbial groups per each anti-microbial active unit is one, e.g., n₁+n₂=1 and m=1 (“non-branched units”).

In some embodiments, the anti-microbial active unit is represented by structure (2):

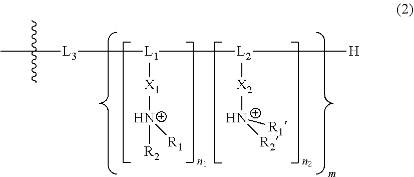

wherein
L₁ is a first linker or a bond;
L₂ is a second linker;
L₃ is a third linker or a bond;
R₁ and R₁′ are each independently alkyl, terpenoid, cycloalkyl, aryl, heterocycle, alkenyl, alkynyl or any combination thereof,
R₂ and R₂′ are each independently alkyl, terpenoid, cycloalkyl, aryl, heterocycle, alkenyl, alkynyl or any combination thereof,
X₁ and X₂ is each independently a bond, alkylene, alkenylene, or alkynylene;
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.
   In another embodiment, the anti-microbial group is —HN(R₁)(R₂)⁺, —HN(R₁)⁺—, —HN(R₁′)(R₂′)⁺ or —HN(R₁′)⁺— (all possibilities are connected covalently to X₁ or X₂).

In another embodiment, the number of the anti-microbial groups per each anti-microbial active unit is at least two, e.g., n₁+n₂≥2 and m≥1 (“branched units”). In another embodiment, the number of the anti-microbial groups per each anti-microbial active unit is one, e.g., n₁+n₂=1 and m=1 (“non-branched units”).

In some embodiments, the anti-microbial active unit is represented by structure (3):

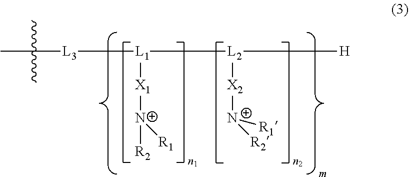

wherein
L₁ is a first linker or a bond;
L₂ is a second linker;
L₃ is a third linker or a bond;
R₁ and R₁′ are each independently alkyl, terpenoid, cycloalkyl, aryl, heterocycle, alkenyl, alkynyl or any combination thereof,
R₂ and R₂′ are each independently alkyl, terpenoid, cycloalkyl, aryl, heterocycle, alkenyl, alkynyl or any combination thereof,
X₁ and X₂ is each independently a bond, alkylene, alkenylene, or alkynylene;
n₁ is each independently an integer between 0 to 200;
n₂ is each independently an integer between 0 to 200;
wherein n₁+n_{2≥}1;
m is an integer between 1 to 200 and the repeating unit is the same or different; and
the notion “ [symbol] ” denotes a covalent bond to an organic or inorganic core.
   In another embodiment, the anti-microbial group is —N(R₁)(R₂), —N(R₁)—, —N(R₁′)(R₂′) or —N(R₁′)— (all possibilities are connected covalently to X₁ or X₂).

In another embodiment, the number of the anti-microbial groups per each anti-microbial active unit is at least two, e.g., n₁+n₂≥2 and m≥1 (“branched units”). In another embodiment, the number of the anti-microbial groups per each anti-microbial active unit is one, e.g., n₁+n₂=1 and m=1 (“non-branched units”).

In another embodiment, the anti-microbial active units of structures (1) to (3) comprise one monomeric unit per one anti-microbial active unit. In another embodiment, the anti-microbial active units of structures (1) to (3) comprise more than one anti-microbial group per one anti-microbial active unit.

### B) Non-Branched Units

In some embodiments, the anti-microbial active unit is represented by structure (4):

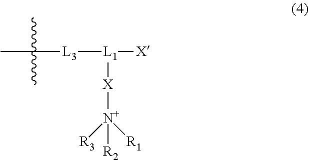

wherein
L₁ is a first linker or a bond;
L₃ is a third linker or a bond;
R₁ is alkyl, terpenoid, cycloalkyl, aryl, heterocycle, alkenyl, alkynyl or any combination thereof;
R₂ is alkyl, terpenoid, cycloalkyl, aryl, heterocycle, alkenyl, alkynyl or any combination thereof;
R₃ is nothing, hydrogen, alkyl, terpenoid moiety, cycloalkyl, aryl, heterocycle, alkenyl, alkynyl or any combination thereof; wherein if R₃ or R₃′ are nothing, the nitrogen is not charged;
X is a bond, alkyl, alkenyl, or alkynyl;
X′ is nothing or hydrogen; and
wherein if L₁ and X are bonds, then the nitrogen is part of an organic or inorganic core;
wherein at least one of R₁, R₂, R₃ is hydrophobic; and
the notion “ [symbol] ” denotes a covalent bond to the organic or inorganic core.
   In another embodiment, the anti-microbial group is {N(R₁)(R₂)(R₃)}⁺ (connected covalently to X).

In some embodiments, the anti-microbial active unit is represented by structure (5):

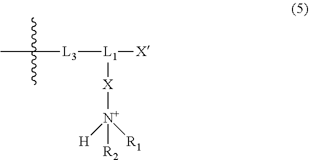

wherein
L₁ is a first linker or a bond;
L₃ is a third linker or a bond;
R₁ is alkyl, terpenoid, cycloalkyl, aryl, heterocycle, alkenyl, alkynyl or any combination thereof;
R₂ is alkyl, terpenoid, cycloalkyl, aryl, heterocycle, alkenyl, alkynyl or any combination thereof;
X is a bond, alkyl, alkenyl or alkynyl;
X′ is nothing or hydrogen; and
wherein if L₁ and X are bonds, then the nitrogen is an integral part of an organic or inorganic core;
wherein at least one of R₁, R₂ is hydrophobic; and
the notion “ [symbol] ” denotes a covalent bond to the organic or inorganic core.

In another embodiment, the anti-microbial group is {HN(R₁)(R₂)}⁺ (connected covalently to X).

In some embodiments, the anti-microbial active unit is represented by structure (6):

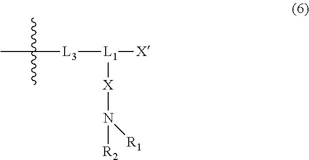

wherein
L₁ is a first linker or a bond;
L₃ is a third linker or a bond;
R₁ is alkyl, terpenoid, cycloalkyl, aryl, heterocycle, alkenyl, alkynyl or any combination thereof;
R₂ is alkyl, terpenoid, cycloalkyl, aryl, heterocycle, alkenyl, alkynyl or any combination thereof;
X is a bond, alkyl, alkenyl, or alkynyl;
X′ is nothing or hydrogen; and
wherein if L₁ and X are bonds, then the nitrogen is an integral part of an organic or inorganic core;
wherein at least one of R₁, R₂ is hydrophobic; and
the notion “ [symbol] ” denotes a covalent bond to the organic or inorganic core.

In another embodiment, the anti-microbial group is {N(R₁)(R₂)}(connected covalently to X).

Specific examples of anti-microbial active units of this invention include:

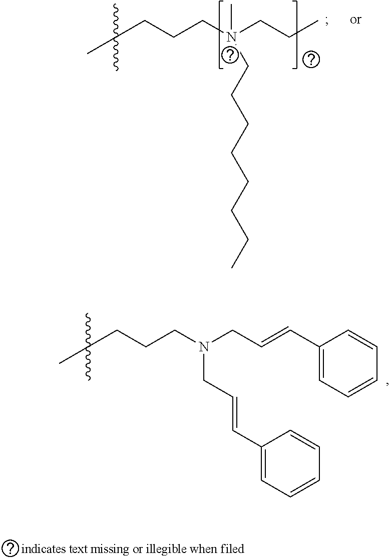

wherein n=1-200; and the notion “ [symbol] ” denotes a covalent bond to an organic or inorganic core. In another embodiment, n=1-3. In another embodiment, n=3-20. In another embodiment, n=20-50. In another embodiment, n=50-100. In another embodiment, n=100-200. In another embodiment, the core is silica or polyhedral oligomeric silsesquioxane (POSS).

In some embodiments, the term “anti-microbial group” and the term “monomeric anti-microbial group” refer to the same and comprise a protonated tertiary amine, a tertiary amine or a quaternary ammonium, as represented by the following formulas:

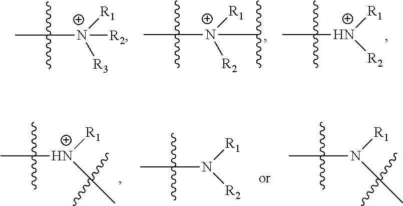

wherein:
R₁ is alkyl, terpenoid, cycloalkyl, aryl, heterocycle, alkenyl, alkynyl or any combination thereof;
R₂ is alkyl, terpenoid, cycloalkyl, aryl, heterocycle, alkenyl, alkynyl or any combination thereof;
R₃ is nothing, hydrogen, alkyl, terpenoid moiety, cycloalkyl, aryl, heterocycle, alkenyl, alkynyl or any combination thereof; wherein if R₃ or R₃′ are nothing, the nitrogen is not charged.

In another embodiment, at least one of R₁, R₂ or R₃ is hydrophobic.

In another embodiment, the number of the anti-microbial groups per each anti-microbial active unit is at least two, e.g., n₁+n₂≥2 and m≥1. In another embodiment, the number of the anti-microbial groups per each anti-microbial active unit is one, e.g., n₁+n₂=1 and m=1.

In another embodiment, the anti-microbial active units of structures (4) to (6) comprise one monomeric unit. In another embodiment, the anti-microbial active units of structures (1) to (3) comprise one or more than one anti-microbial groups.

In another embodiment, the anti-microbial active unit of structures (1) to (6) is covalently bonded to an inorganic core. In another embodiment, the anti-microbial active unit of structure (1) to (6) is covalently bonded to an organic core. In another embodiment, the organic core is a polymeric organic core. In another embodiment, the core is inert. In one embodiment, the anti-microbial active units of this invention represented by structures (1)-(3) comprise an anti-microbial group of —⁺N(R₁)(R₂)(R₃), —⁺N(R₁)(R₂)—, —⁺NH(R₁)(R₂), —⁺NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′) or —N(R₁′)—. In one embodiment R₁ and/or R₁′, R₂ and/or R₂′ and R₃ and/or R₃′ are the same or different and are independently alkyl, terpenoid, cycloalkyl, aryl, heterocycle, alkenyl, alkynyl or any combination thereof. In another embodiment, R₁, R₂ and R₃ are independently an alkyl. In another embodiment, R₁ and/or R₁′, R₂ and/or R₂′ and R₃ and/or R₃′ are independently a terpenoid. In another embodiment, R₁ and/or R₁′, R₂ and/or R₂′ and R₃ and/or R₃′ are independently a cycloalkyl. In another embodiment, R₁ and/or R₁′, R₂ and/or R₂′ and R₃ and/or R₃′ are independently an aryl. In another embodiment, R₁ and/or R₁′, R₂ and/or R₂′ and R₃ and/or R₃′ are independently a heterocycle. In another embodiment, R₁ and/or R₁′, R₂ and/or R₂′ and R₃ and/or R₃′ are independently an alkenyl. In another embodiment, R₁ and/or R₁′, R₂ and/or R₂′ and R₃ and/or R₃′ are independently an alkynyl. In another embodiment, R₃ is nothing. In another embodiment, R₃ and/or R₃′ is hydrogen. In another embodiment at least one of R₁ and/or R₁′, R₂ and/or R₂′ and R₃ and/or R₃′ is hydrophobic alkyl, terpenoid, cycloalkyl, aryl, heterocycle, alkenyl, alkynyl or any combination thereof. Each represents a separate embodiment of this invention.

In another embodiment R₁ and R₁′ are the same. In another embodiment R₂ and R₂′ are the same. In another embodiment R₃ and R₃′ are the same. In another embodiment R₁ and R₁′ are different. In another embodiment R₂ and R₂′ are different. In another embodiment R₃ and R₃′ are different.

In one embodiment, at least one of R₁, R₂ and R₃ and/or at least one of R₁′, R₂′ and R₃′ of structure (1) is hydrophobic. In one embodiment, at least one of R₁ and R₂ and/or at least one of R₁′ and R₂′ of structures (2) and (3) is hydrophobic.

The term “hydrophobic” refers to an alkyl, alkenyl or alkynyl having at least four carbons, or the term hydrophobic refers to terpenoid, to cycloalkyl, aryl or heterocycle having at least six carbons. Each possibility represents a separate embodiment of this invention

In one embodiment, at least one of R₁, R₂ and R₃ and/or at least one of R₁′, R₂′ and R₃′ of structure (1) is a C₄-C₂₄ alkyl, C₄-C₂₄ alkenyl, C₄-C₂₄ alkynyl or a terpenoid. In one embodiment, at least one of R₁ and R₂ and/or at least one of R₁′ and R₂′ of structures (2) and (3) is a C₄-C₂₄ alkyl, C₄-C₂₄ alkenyl, C₄-C₂₄ alkynyl or a terpenoid. Each possibility represents a separate embodiment of this invention.

In one embodiment, at least one of R₁, R₂ and R₃ and/or at least one of R₁′, R₂′ and R₃′ of structure (1) is a C₄-C₈ alkyl, C₄-C₈ alkenyl, C₄-C₈ alkynyl or a terpenoid. In one embodiment, at least one of R₁ and R₂ and/or at least one of R₁′ and R₂′ of structures (2) and (3) is a C₄-C₈ alkyl, C₄-C₈ alkenyl, C₄-C₈ alkynyl or a terpenoid. Each possibility represents a separate embodiment of this invention.

In one embodiment, R₁ and/or R₁′ of structures (1) to (6) is a terpenoid. In another embodiment, R₁ and/or R₁′ is a terpenoid and R₂ and/or R₂′ is a C₁-C₄ alkyl. In another embodiment, the core is an organic polymeric core, R₃ and/or R₃′ is nothing and R₁ and/or R₁′ is a terpenoid. In another embodiment, the core is an organic polymeric core, R₃ and/or R₃′ is a hydrogen and R₁ and/or R₁′ is a terpenoid. In another embodiment, the core is an inorganic core, R₃ and/or R₃′ is nothing and R₁ and/or R₁′ is a terpenoid. In another embodiment, the core is an inorganic core, R₃ and/or R₃′ is a hydrogen and R₁ and/or R₁′ is a terpenoid. In another embodiment, the core is an inorganic core, R₃ and/or R₃′ is a C₁-C₂₄ alkyl, terpenoid, cycloalkyl, aryl, heterocycle, a conjugated C₁-C₂₄ alkyl, C₁-C₂₄ alkenyl, C₁-C₂₄ alkynyl or any combination thereof and R₁ and/or R₁′ is a terpenoid.

In one embodiment, the particles of this invention comprise an anti-microbial active unit and an inert core, wherein the anti-microbial active unit and the core are linked indirectly.

In some embodiments L₁, L₂ or L₃ is each independently the same or a different linker. In some embodiments, L₁, L₂ and L₃ are connected to each other, in any possible way. In some embodiment, L₃ is nothing (or a bond) and L₁ or L₂ is connected to the core covalently. In another embodiment, L₃ is connected to the core covalently and L₁ or L₂ is connected to L₃. In another embodiment, L₁ is connected to X, X′ and L₃ or core. In another embodiment, a “linker” comprises any possible chemical moiety capable of connecting at least two other chemical moieties which are adjacent to such linker. In another embodiment, the monomeric unit of the anti-microbial active unit comprises a first and/or second linker/s (L₁ or L₂) and an anti-microbial group. In another embodiment, L₁ and/or L₂ are/is the backbone of the anti-microbial active unit. In another embodiment, the monomeric unit of the anti-microbial active unit comprises a first and/or second linker/s (L₁ or L₂) and an anti-microbial group. In another embodiment, L₁ and/or L₂ are/is the backbone (they are e.g., alkylene, polypeptide or oligosiloxane (—Si(OH)₂—O— or —Si(CH₃)₂—O—) moieties) of the anti-microbial active unit.

In some embodiments, the linker comprises a functional group. In another embodiment, the linker comprises two (same or different) functional groups. In another embodiment, the functional group comprises phosphate, phosphonate, siloxane, silane, ether acetal, amide, amine, anhydride, ester, ketone, or aromatic ring or rings functionalized with any of the preceding moieties. Each possibility represents a separate embodiment of this invention.

In another embodiment, L₁ or L₂ is a C1 to C18 alkylene, alkenylene, alkynylene or aryl substituted with at least one carboxyl moiety, wherein the carboxyl end is attached to the core. This linker may be derived from a C1 to C18 alkylene substituted with at least one carboxyl moiety and having an amino end which is modified to anti-microbial group [—⁺N(R₁)(R₂)(R₃), —⁺N(R₁)(R₂)—, —⁺NH(R₁)(R₂), —⁺NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′) or —N(R₁′)— (defined in structures (1) to (6))]. This linker may be derived from an amino acid of natural or synthetic source having a chain length of between 2 and 18 carbon atoms (polypeptide), or an acyl halide of said amino acid. Non-limiting examples for such amino acids are 18-amino octadecanoic acid and 18-amino stearic acid. In another embodiment, L₁ or L₂ is a C1 to C18 alkylene substituted with at least one amine or amide moiety.

In another embodiment, L₁, L₂, L₃ or any combination thereof is a C1 to C18 alkylene, alkenylene, alkynylene or aryl. This linker may be derived from a di-halo alkylene, which is functionalized at each end with the core and anti-microbial group, respectively, by replacement of the halogen moiety to a functional group that binds to the core and replacement of the halogen moiety to obtain [—⁺N(R₁)(R₂)(R₃), —⁺N(R₁)(R₂)—, —⁺NH(R₁)(R₂), —⁺NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′) or —N(R₁′)— (defined in structures (1) to (6))].

In another embodiment, L₁, L₂, L₃ or any combination thereof is an aromatic group derived from non-limiting examples of 4,4-biphenol, dibenzoic acid, dibenzoic halides, dibenzoic sulphonates, terephthalic acid, terephthalic halides, and terephthalic sulphonates. This linker is functionalized with the core and anti-microbial group, respectively, through the functional group thereof (e.g., hydroxyl, carboxy or sulfonate). In another embodiment, this linker is directly attached to the core at one end or indirectly, via a third linker (L₃) and is modified at the other end to anti-microbial group [—⁺N(R₁)(R₂)(R₃), —⁺N(R₁)(R₂)—, —+NH(R₁)(R₂), —⁺NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′) or —N(R₁′)— (defined in structures (1) to (6))].

In another embodiment, L₁, L₂, L₃ or any combination thereof, is a siloxane or silane group derived and/or selected from non-limiting examples of trialkoxyalkylsilane, trialkoxyarylsilane, trihaloalkylsilane, trihaloarylsilane, 3-aminopropyltriethoxysilane (APTES), (3-glycidyloxypropyl)trimethoxysilane and N-2-aminoethyl-3-aminopropyl trimethoxysilane. This linker is functionalized with the core and anti-microbial group, respectively, through the functional group thereof (e.g., hydroxyl, siloxane, carboxy, amide or sulfonate). In another embodiment, this linker is directly attached to the core at one end directly or indirectly, via a third linker (L₃) and is modified at the other end to anti-microbial group [—⁺N(R₁)(R₂)(R₃), —⁺N(R₁)(R₂)—, —⁺NH(R₁)(R₂), —⁺NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′) or —N(R₁′)— (defined in structures (1) to (6))].

In one embodiment, the anti-microbial group of this invention may be selected from: (a) a tertiary amine (e.g., R₃ and/or R₃′ is nothing) or tertiary ammonium (e.g., R₃ and/or R₃′ is hydrogen) comprising at least one terpenoid moiety (b) a quaternary ammonium group comprising at least one terpenoid moiety (c) a quaternary ammonium group, comprising at least one alkyl group having from 4 to 24 carbon atoms; and (d) a tertiary amine (e.g., R₃ and/or R₃′ is nothing) or tertiary ammonium (e.g., R₃ and/or R₃′ is hydrogen) comprising at least one alkyl group having from 4 to 24 carbon atoms. Each possibility represents a separate embodiment of this invention.

This linker is functionalized with the core and anti-microbial active unit, respectively, through the functional group thereof (e.g., hydroxyl, siloxane, carboxy, amide or sulfonate). In another embodiment, this linker is directly attached to the core at one end or indirectly, via a third linker (L₃) and is modified at the other end to anti-microbial group [—⁺N(R₁)(R₂)(R₃), —⁺N(R₁)(R₂)—, —⁺NH(R₁)(R₂), —⁺NH(R₁)—, —N(R₁)(R₂), —N(R₁), —⁺N(R₁′)(R₂′)(R₃′), —⁺N(R₁′)(R₂′)—, —NH(R₁′)(R₂′), —⁺NH(R₁′)—, —N(R₁′)(R₂′) or —N(R₁′)— (defined in structures (1) to (6))].

In another embodiment, a monomeric unit (as described in e.g., FIGS. 2A-2B and formulas 1-6) within the anti-microbial active unit of this invention is represented by the structure of formula IA:

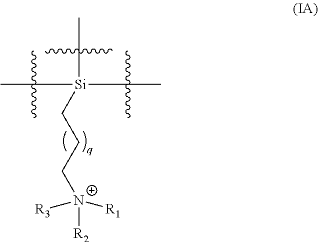

wherein
R₁ and R₂ are independently linear or branched alkyl, terpenoid, cycloalkyl, aryl, heteroaryl, alkenyl, alkynyl or any combination thereof, and
R₃ is nothing, linear or branched alkyl, terpenoid, cycloalkyl, aryl, heteroaryl, alkenyl, alkynyl or any combination thereof, wherein if R₃ is nothing, the nitrogen is not charged
q is an integer between 0 and 16;
wherein said monomeric unit is chemically bound to the surface of an inorganic core directly or via a third linker (L₃).

In another embodiment, a monomeric unit (as described in e.g., FIGS. 2A-2B and formulas 1-6) within the anti-microbial active unit of this invention is represented by the structure of formula IB:

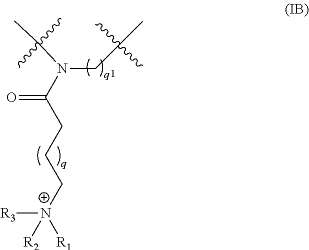

wherein
R₁ and R₂ are independently linear or branched alkyl, terpenoid, cycloalkyl, aryl, heteroarylalkenyl, alkynyl or any combination thereof; and
R₃ is nothing, linear or branched alkyl, terpenoid, cycloalkyl, aryl, heteroaryl, alkenyl, alkynyl or any combination thereof; wherein if R₃ in nothing, the nitrogen is not charged
q and q¹ are independently an integer between 0 and 16;
wherein said monomeric unit is chemically bound to the surface of an inorganic core directly or via a third linker (L₃).

In another embodiment, a linker molecule which may be used in the processes of preparing the anti-microbial particles of this invention is represented by the structure of formula IC:

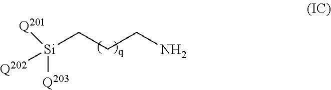

wherein
Q²⁰¹, Q²⁰² and Q²⁰³ are independently selected from the group consisting of alkoxy, methyl, ethyl, hydrogen, sulfonate and halide, wherein at least one of Q²⁰¹, Q²⁰² and Q²⁰³ is selected from ethoxy, methoxy, sulfonate (e.g., mesyl, tosyl) and halide; and
q is an integer between 0 and 16;
the linker molecule is capable of being chemically bound to the surface of the inorganic core through the silicon atom; and
the anti-microbial group is introduced by functionalizing the primary amine to obtain an anti-microbial active tertiary amine or quaternary ammonium group containing at least one terpenoid group, as described above.

In another embodiment, a linker molecule which may be used in the processes of preparing the anti-microbial particles of this invention is represented by the structure of formula ID:

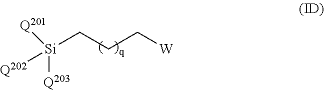

wherein
Q²⁰¹, Q²⁰² and Q²⁰³ are independently selected from the group consisting of alkoxy, methyl, ethyl, hydrogen, sulfonate and halide, wherein at least one of Q²⁰¹, Q²⁰² and Q²⁰³ is selected from ethoxy, methoxy, sulfonate (e.g., mesyl, tosyl) and halide;
W is selected from the group consisting of NH₂, halide, sulfonate and hydroxyl; and
q is an integer between 0 and 16;
said linker is capable of being chemically bound to the surface of said inorganic core through the silicon atom; and
the anti-microbial group is introduced by substituting the group W with an anti-microbial group, or converting the group W to an anti-microbial group.

### Anti-Microbial Groups Comprising One Long Alkyl Group.

In accordance with another embodiment, the anti-microbial group of this invention [—⁺N(R₁)(R₂)(R₃), —⁺N(R₁)(R₂)—, —⁺NH(R₁)(R₂), —NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′) or —N(R₁′)— (defined in structures (1) to (6))] is a quaternary ammonium group, a tertiary amine or a tertiary ammonium, the nitrogen atom of each amine/ammonium group having at least one bond X₁ or X₂, at least one bond to an alkyl group having from 4 to 24 carbon atoms (R₁ and/or R₁′). In another embodiment, the nitrogen atom of each amine/ammonium group having one bond to the core, one bond to an alkyl group having from 4 to 24 carbon atoms (R₁ and/or R₁′).

In some embodiments, the nitrogen atom of each quaternary ammonium or tertiary ammonium group has (i) at least one bond to X₁ or X₂; and (ii) at least one bond to the alkyl group having from 4 to 24 carbon atoms.

In some embodiments, the anti-microbial group of formula (1) to (6) is selected from: (a) a tertiary amine (R₃ and/or R₃′ is nothing) or tertiary ammonium (R₃ and/or R₃′ is H), wherein the nitrogen atom of each tertiary amine/ammonium having at least one bond to X₁ or X₂ and one bond to the alkyl group having from 4 to 24 carbon atoms; (b) a tertiary amine (R₃ and/or R₃′ is nothing), or tertiary ammonium (R₃ and/or R₃′ is H), wherein the nitrogen atom of each tertiary amine/ammonium having one bond to X₁ or X₂ and two bonds to alkyl groups having from 4 to 24 carbon atoms which may be the same or different from each other, or a salt of said tertiary amine; (c) a quaternary ammonium group wherein the nitrogen atom of each quaternary ammonium group having at least one bond to X₁ or X₂ and one or two bonds to the alkyl groups having from 4 to 24 carbon atoms which may be the same or different from each other. Each possibility represents a separate embodiment of this invention.

The term “quaternary ammonium group” refers to a group of atoms consisting of a nitrogen atom with four substituents (different than hydrogen) attached thereto. In another embodiment, a “quaternary ammonium group” refers to a group of atoms consisting of a nitrogen atom with four groups wherein each of the group is attached to the nitrogen through a carbon atom. The term “long alkyl group” or chain refers to such an alkyl group or chain which is substituted on the nitrogen atom of the quaternary ammonium group and which has between 4 and 24 carbon atoms. In some embodiments, the alkyl group is an alkyl group having 4 to 18 carbon atoms. In some embodiments, the alkyl group is an alkyl group having 4 to 8 carbon atoms. In some embodiments, the alkyl group is an alkyl group having 4 to 10 carbon atoms. In other embodiments, the alkyl group is an alkyl group having 6, 7, or 8 carbon atoms, with each possibility representing a separate embodiment of this invention.

### C) Thermally Stable Anti-Microbial Units

In one embodiment, the anti-microbial active unit is represented by structure (I):

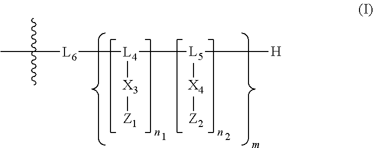

wherein
the core is an organic polymer or an inorganic material;
L₄ is a first linker or a bond;
L₅ is a second linker;
L₆ is a third linker or a bond;
Z₁ is

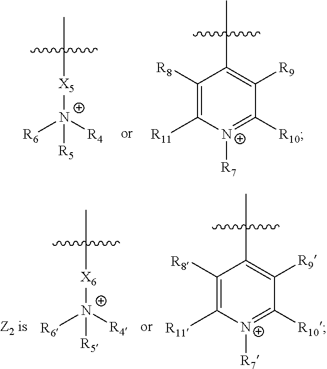

R₄ and R₄′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ and R₅′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or
any combination thereof,
R₆ and R₆′ are each independently absent, methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof,
R₇ and R₇′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof,
R₇ and R₇′ are each independently H, alkyl, terpenoid moiety, cycloalkyl, aryl, heterocycle, a conjugated alkyl, alkenyl, alkynyl or any combination thereof,
R₉ and R₉′ are each independently H, alkyl, terpenoid moiety, cycloalkyl, aryl, heterocycle, a conjugated alkyl, alkenyl, alkynyl or any combination thereof,
R₁₀ and R₁₀′ are each independently H, alkyl, terpenoid moiety, cycloalkyl, aryl, heterocycle, a conjugated alkyl, alkenyl, alkynyl or any combination thereof,
R₁₁ and R₁₁′ are each independently H, alkyl, terpenoid moiety, cycloalkyl, aryl, heterocycle, a conjugated alkyl, alkenyl, alkynyl or any combination thereof,
X₃ and X₄ are each independently a bond, alkylene, arylene, alkenylene, alkynylene or any combination thereof,
X₅ and X₆ are each independently a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof,
R is alkyl, aryl, cycloalkyl, heterocycle or any combination thereof,
n₁ is each independently an integer between 0 to 200;
n₂ is each independently an integer between 0 to 200;
wherein n₁+n₂≥1;
m is an integer between 1 to 200 and the repeating unit is the same or different; and the notion “ [symbol] ” denotes a covalent bond to an organic or inorganic core.
   In another embodiment, the anti-microbial group is Z₁ or Z₂ (both possibilities are connected covalently to X₃ or X₄).

In another embodiment, provided that Z₁ or Z₂ comprises an ammonium nitrogen (not pyridinium)—in each of the anti-microbial active units only one moiety on the ammonium may have beta hydrogens available for Hofmann elimination. In another embodiment, provided that Z₁ or Z₂ comprises an ammonium nitrogen (not pyridinium)—in each of the anti-microbial active units two moieties on the ammonium may have beta hydrogens available for Hofmann elimination. In another embodiment, beta hydrogens available for Hofmann elimination are those which are found on beta (compared to the ammonium nitrogen) aliphatic carbon and can be eliminated to release an olefin and a tertiary amine.

In another embodiment, the anti-microbial active unit is represented by structure (IE):

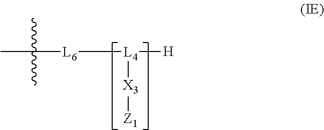

wherein
the core is an organic polymer or an inorganic material;
L₄ is a first linker or a bond;
L₆ is a third linker or a bond;
Z₁ is

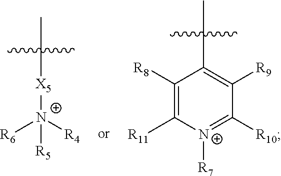

R₄ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof,
R₆ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof,
R₇ is methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof,
R₈ is H, alkyl, terpenoid moiety, cycloalkyl, aryl, heterocycle, a conjugated alkyl, alkenyl, alkynyl or any combination thereof,
R₉ is H, alkyl, terpenoid moiety, cycloalkyl, aryl, heterocycle, a conjugated alkyl, alkenyl, alkynyl or any combination thereof,
R₁₀ is H, alkyl, terpenoid moiety, cycloalkyl, aryl, heterocycle, a conjugated alkyl, alkenyl, alkynyl or any combination thereof,
R₁₁ is H, alkyl, terpenoid moiety, cycloalkyl, aryl, heterocycle, a conjugated alkyl, alkenyl, alkynyl or any combination thereof,
X₃ is a bond, alkylene, arylene, alkenylene, alkynylene or any combination thereof,
X₅ is a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof,
R is alkyl, aryl, cycloalkyl, heterocycle or any combination thereof, and
the notion “ [symbol] ” denotes a covalent bond to an organic or inorganic core.
   In another embodiment, the anti-microbial group is Z₁ (connected covalently to X₃).

In another embodiment, provided that Z₁ comprises an ammonium nitrogen (not pyridinium)—in each of the anti-microbial active units only one moiety on the ammonium may have beta hydrogens available for Hofmann elimination. In another embodiment, provided that Z₁ or Z₂ comprises an ammonium nitrogen (not pyridinium)—in each of the anti-microbial active units two moieties on the ammonium may have beta hydrogens available for Hofmann elimination.

In another embodiment, the anti-microbial active unit is represented by structure (II):

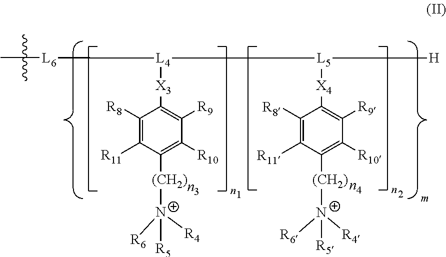

wherein
the core is an organic polymer or an inorganic material;
L₄ is a first linker or a bond;
L₅ is a second linker;
L₆ is a third linker or a bond;
R₄ and R₄′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₅ and R₅′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof,
R₆ and R₆′ are each independently methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof,
R₇ and R₇′ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof,
R₈ and R₈′ are each independently H, alkyl, terpenoid moiety, cycloalkyl, aryl, heterocycle, a conjugated alkyl, alkenyl, alkynyl or any combination thereof,
R₉ and R₉′ are each independently H, alkyl, terpenoid moiety, cycloalkyl, aryl, heterocycle, a conjugated alkyl, alkenyl, alkynyl or any combination thereof,
R₁₀ and R₁₀′ are each independently H, alkyl, terpenoid moiety, cycloalkyl, aryl, heterocycle, a conjugated alkyl, alkenyl, alkynyl or any combination thereof;
R₁₁ and R₁₁′ are each independently H, alkyl, terpenoid moiety, cycloalkyl, aryl, heterocycle, a conjugated alkyl, alkenyl, alkynyl or any combination thereof;
X₃ and X₄ are each independently a bond, alkylene, arylene, alkenylene, alkynylene or any combination thereof;
R is alkyl, aryl, cycloalkyl, heterocycle or any combination thereof;
n₁ is each independently an integer between 0 to 200;
n₂ is each independently an integer between 0 to 200;
n₃ and n₄ are each independently 0 or 1;
wherein n₁+n₂≥1; and
m is an integer between 1 to 200 and the repeating unit is the same or different; and
the notion “ [symbol] ” denotes a covalent bond to an organic or inorganic core.
   In another embodiment, the anti-microbial group is —N(R₄)(R₅)(R₆)⁺ or —N(R₄′)(R₅′)(R₆′)⁺ (all possibilities are connected covalently to {(CH₂)n₃} or {(CH₂)n₄}).

In another embodiment, in each of the anti-microbial active units only one moiety on the ammonium may have beta hydrogens available for Hofmann elimination.

In another embodiment, the anti-microbial active unit is represented by structure (III):

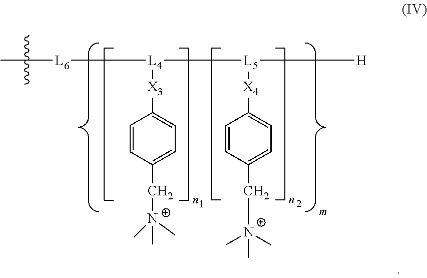

wherein
the core is an organic polymer or an inorganic material;
L₄ is a first linker or a bond;
L₅ is a second linker;
L₆ is a third linker or a bond;
X₃ and X₄ are each independently a bond, alkylene, arylene, alkenylene, alkynylene or any combination thereof;
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.

In another embodiment, the anti-microbial group is —N(CH₃)₃⁺ (connected covalently to {CH₂}).

In another embodiment, the anti-microbial active unit is represented by structure (IV):

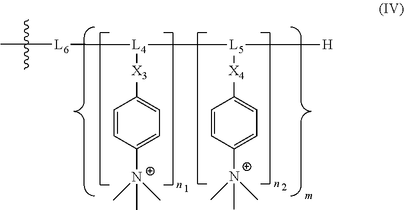

wherein
the core is an organic polymer or an inorganic material;
L₄ is a first linker or a bond;
L₅ is a second linker;
L₆ is a third linker or a bond;
X₃ and X₄ are each independently a bond, alkylene, arylene, alkenylene, alkynylene or any combination thereof;
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.

In another embodiment, the anti-microbial group is —N(CH₃)₃⁺ (connected covalently to {C₆H₄}).

In another embodiment, the anti-microbial active unit is represented by structure (V):

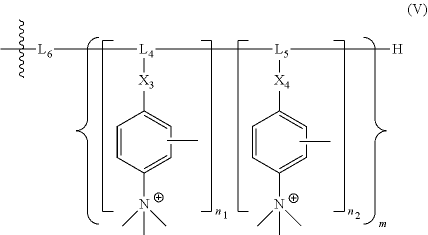

wherein
the core is an organic polymer or an inorganic material;
L₄ is a first linker or a bond;
L₅ is a second linker;
L₆ is a third linker or a bond;
X₃ and X₄ are each independently a bond, alkylene, arylene, alkenylene, alkynylene or any combination thereof,
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.

In another embodiment, the anti-microbial group is —N(CH₃)₃⁺ (connected covalently to {C₆H₄}).

In another embodiment, the anti-microbial active unit is represented by structure (VI):

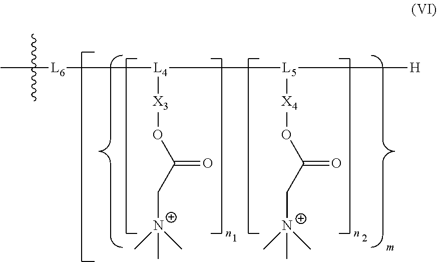

wherein
the core is an organic polymer or an inorganic material;
L₄ is a first linker or a bond;
L₅ is a second linker;
L₆ is a third linker or a bond;
X₃ and X₄ are each independently a bond, alkylene, arylene, alkenylene, alkynylene or any combination thereof;
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.

In another embodiment, the anti-microbial group is —N(CH₃)₃⁺ (connected covalently to {CH₂}).

In another embodiment, the anti-microbial active unit is represented by structure (VII):

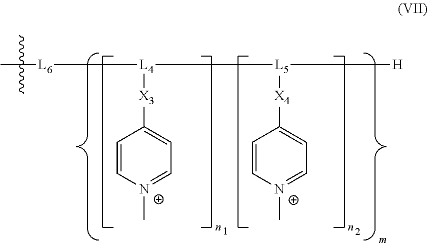

wherein
the core is an organic polymer or an inorganic material;
L₄ is a first linker or a bond;
L₅ is a second linker;
L₆ is a third linker or a bond;
X₃ and X₄ are each independently a bond, alkylene, arylene, alkenylene, alkynylene or any combination thereof,
n₁ is each independently an integer between 0 to 200;
n₂ is each independently an integer between 0 to 200;
wherein n₁+n₂≥1;
m is an integer between 1 to 200 and the repeating unit is the same or different; and
the notion “ [symbol] ” denotes a covalent bond to an organic or inorganic core.

In another embodiment, the anti-microbial group is

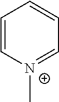

(connected covalently to X₃ or X₄).

In some embodiments, the anti-microbial particles comprising the anti-microbial active units of structures (I), (IE) and (II)-(VII) have high thermal stability. Without being bound by any mechanism or theory, it is suggested that the high stability stems from lack of available beta (β) hydrogens on the ammonium or a low number thereof, thus reducing the possibility of having a Hofmann elimination which in turn gives rise to reduced thermal stability.

In some embodiments, the term “anti-microbial group” and the term “monomeric anti-microbial group” refer to the same and comprise a quaternary ammonium and/or a pyridinium, as represented by the following formulas:

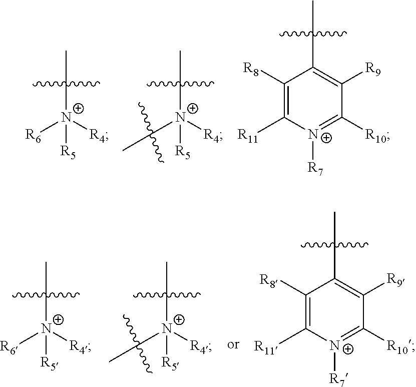

wherein:
R₄-R₁₁ and R₄′-R₁₁′ are as described hereinabove.

In another embodiment, the number of the anti-microbial groups per each anti-microbial active unit is at least two, e.g., n₁+n₂≥2 and m≥1. In another embodiment, the number of the anti-microbial groups per each anti-microbial active unit is one, e.g., n₁+n₂=1 and m=1.

In another embodiment, the anti-microbial active unit of structure (IE) comprise one monomeric unit per one anti-microbial active unit. In another embodiment, the anti-microbial active units of structures (I) and (II) to (VII) comprise one or more than one anti-microbial group per one anti-microbial active unit.

In another embodiment, the anti-microbial active units of structures (I), (IE) and (II)-(VII) has an inorganic core. In another embodiment, the anti-microbial active units of structure (I), (IE) and (II)-(VII) are connected to an organic core. In another embodiment, the organic core is a polymeric organic core. In another embodiment, the core is inert.

In one embodiment, Z₁ is

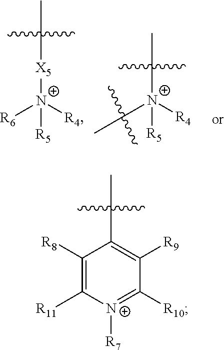

wherein X₅ and R₄-R₁₁ are as described hereinbelow. Each possibility represents a separate embodiment of this invention.

In one embodiment, Z₂ is

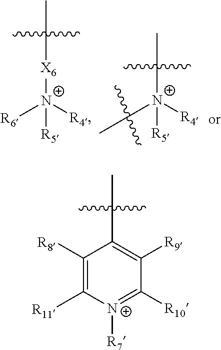

wherein X₆ and R_{4′}-R_{11′} are as described hereinbelow. Each possibility represents a separate embodiment of this invention.

In one embodiment, R₄ and/or R₄′, R₅ and/or R₅′ and R₇ and/or R₇′ are the same or different and are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, wherein R is described hereinbelow. Each possibility represents a separate embodiment of this invention.

In one embodiment, R₆ and R₆′ are each independently absent, methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof, Each possibility represents a separate embodiment of this invention.

In one embodiment, R₈ and/or R₈′, R₉ and/or R₉′, R₁₀ and/or R₁₀′ and R₁₁ and/or R₁₁′ are the same or different and are independently H, alkyl, terpenoid moiety, cycloalkyl, aryl, heterocycle, a conjugated alkyl, alkenyl, alkynyl or any combination thereof. Each possibility represents a separate embodiment of this invention.

In one embodiment, X₃ and/or X₄ are the same or different and are independently a bond, alkylene, arylene, alkenylene, alkynylene or any combination thereof. Each possibility represents a separate embodiment of this invention.

In one embodiment, X₅ and X₆ are each independently a bond, —O—C(═O)—, methylene, —O—C(═O)—CH₂—, 2,2-disubstituted C₂-C₂₀ alkylene, arylene, phenylene, benzylene, cycloalkylene, a heterocycle, a conjugated alkylene, a terpenoid moiety, 1-alkenylene, 1-alkynylene, 2-alkenylene, 2-alkynylene or any combination thereof. Each possibility represents a separate embodiment of this invention.

In one embodiment, R is alkyl, aryl, cycloalkyl, heterocycle or any combination thereof. Each possibility represents a separate embodiment of this invention.

In another embodiment R₄ and R₄′ are the same. In another embodiment R₅ and R₅′ are the same. In another embodiment R₆ and R₆′ are the same. In another embodiment R₇ and R₇′ are the same. In another embodiment R₈ and R₈′ are the same. In another embodiment R₉ and R₉′ are the same. In another embodiment R₁₀ and R₁₀′ are the same. In another embodiment R₁₁ and R₁₁′ are the same. In another embodiment X₃ and X₄ are the same. In another embodiment X₅ and X₆ are the same. In another embodiment R₄ and R₄′ are different. In another embodiment R₅ and R₅′ are different. In another embodiment R₆ and R₆′ are different. In another embodiment R₇ and R₇′ are different. In another embodiment R₈ and R₈′ are different. In another embodiment R₉ and R₉′ are different. In another embodiment R₁₀ and R₁₀′ are different. In another embodiment R₁₁ and R₁₁′ are different. In another embodiment X₃ and X₄ are different. In another embodiment X₅ and X₆ are different.

In another embodiment, at least one of R₄, R₅ and R₆ and/or at least one of R₄′, R₅′ and R₆′ of structure (I) is/are hydrophobic.

In another embodiment, at least one of R₆, R₈-R₁₁ and X₅ and/or at least one of R₆°, R_{8′}-R_{11′} and X₆ of structure (I) is a terpenoid. Each possibility represents a separate embodiment of this invention.

In one embodiment, n₃ and n₄ of structure (II) are each independently 0 or 1. Each possibility represents a separate embodiment of this invention.

In some embodiments L₄, L₅ or L₆ is each independently the same or a different linker. In some embodiments, L₄, L₅ and L₆ are connected to each other, in any possible way. In some embodiment, L₆ is nothing (or a bond) and L₄ or L₅ is connected to the core covalently. In another embodiment, L₆ is connected to the core covalently and L₄ or L₅ is connected to L₆. In another embodiment, L₄ is connected to X₃, L₅ and L₆ or core. In another embodiment, a “linker” comprises any possible chemical moiety capable of connecting at least two other chemical moieties which are adjacent to such linker. In another embodiment, the monomeric unit of the anti-microbial active unit comprises a first and/or second linker/s (L₄ or L₅) and an anti-microbial group. In another embodiment, L₄ and/or L₅ are/is the backbone (they are e.g., alkylene, polypeptide or oligosiloxane (—Si(OH)₂—O— or —Si(CH₃)₂—O—) moieties) of the anti-microbial active unit. In some embodiments, the linker comprises a functional group. In another embodiment, the linker comprises two (same or different) functional groups. In another embodiment, the functional group comprises phosphate, phosphonate, siloxane, silane, ether, acetal, hydroxyl, amide, amine, anhydride, ester, ketone, or aromatic ring or rings functionalized with any of the preceding moieties. Each possibility represents a separate embodiment of this invention.

In another embodiment, L₄, L₅, L₆, X₃, X₄, X₅, X₆ or any combination thereof is a C1 to C18 alkylene, alkenylene, alkynylene or aryl substituted with at least one carboxyl moiety, wherein the carboxyl end is attached to the core. It may be derived from a C1 to C18 alkylene substituted with at least one carboxyl moiety and having an amino end which is modified to anti-microbial group [—⁺N(R₄)(R₅)(R₆), —⁺N(R₄′)(R₅′)(R₆′),

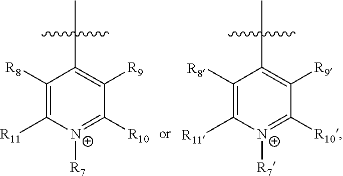

defined in structures (I) and (IE)]. This linker may be derived from an amino acid of natural or synthetic source having a chain length of between 2 and 18 carbon atoms (polypeptide), or an acyl halide of said amino acid. Non-limiting examples for such amino acids are 18-amino octadecanoic acid and 18-amino stearic acid. In another embodiment, L₄, L₅, L₆, X₃, X₄, X₅, X₆ or any combination thereof is a C1 to C18 alkylene substituted with at least one amine, amide or pyridinium

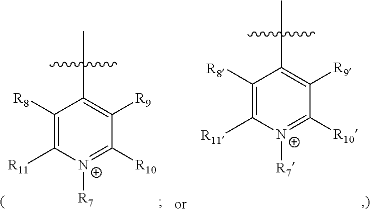

moiety.

In another embodiment, L₄, L₅, L₆, X₃, X₄, X₅, X₆ or any combination thereof is a C1 to C18 alkylene, alkenylene, alkynylene, arylene or aryl. This linker may be derived from a di-halo alkylene or di-haloarylene, which is functionalized at each end with the core and anti-microbial group, respectively, by replacement of the halogen moiety to a functional group that binds to the core and replacement of the halogen moiety to obtain —⁺N(R₄)(R₅)(R₆) or —⁺N(R₄′)(R₅′)(R₆′), which are defined in structures (I) to (II).

In another embodiment, L₄, L₅, L₆, X₃, X₄, X₅, X₆ or any combination thereof is an aromatic group derived from non-limiting examples of 4,4-biphenol, dibenzoic acid, dibenzoic halides, dibenzoic sulphonates, terephthalic acid, tetrphthalic halides, and terephthalic sulphonates. This linker is functionalized with the core and anti-microbial group, respectively, through the functional group thereof (e.g., hydroxyl, carboxy or sulfonate). In another embodiment, this linker is directly attached to the core at one end or indirectly, via a third linker (L) and is modified at the other end to anti-microbial group [—⁺N(R₄)(R₅)(R₆), —⁺N(R₄′)(R₅′)(R₆′),

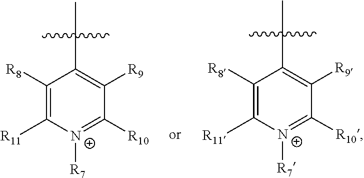

defined in structures (I) and (IE)].

In another embodiment, L₄, L₅, L₆, X₃, X₄, X₅, X₆ or any combination thereof, is a siloxane or silane group derived and/or selected from non-limiting examples of trialkoxyalkylsilane, trialkoxyarylsilane, trihaloalkylsilane, trihaloarylsilane, 3-aminopropyltriethoxysilane (APTES), (3-glycidyloxypropyl)trimethoxysilane and N-2-aminoethyl-3-aminopropyl trimethoxysilane. This linker is functionalized with the core and anti-microbial group, respectively, through the functional group thereof (e.g., hydroxyl, siloxane, carboxy, amide or sulfonate). In another embodiment, this linker is directly attached to the core at one end directly or indirectly, via a third linker (L₆) and is modified at the other end to anti-microbial group [—⁺N(R₄)(R₅)(R₆), —⁺N(R₄′)(R₅′)(R₆′),

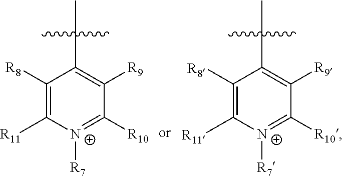

defined in structures (I) and (IE)].

This linker is functionalized with the core and anti-microbial group, respectively, through the functional group thereof (e.g., hydroxyl, siloxane, carboxy, amide or sulfonate). In another embodiment, this linker is directly attached to the core at one end or indirectly, via a third linker (L) and is modified at the other end to anti-microbial active [—⁺N(R₄)(R₅)(R₆), —⁺N(R₄′)(R₅′)(R₆′),

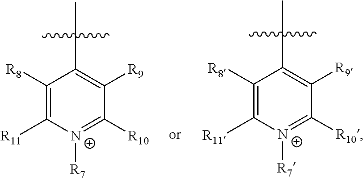

defined in structures (I) and (IE)].

In another embodiment, a monomeric unit (as described in e.g., FIGS. 2A-2B and formulas IE and I-VII) within the anti-microbial active unit of this invention is represented by the structure of formula IF1 or IF2:

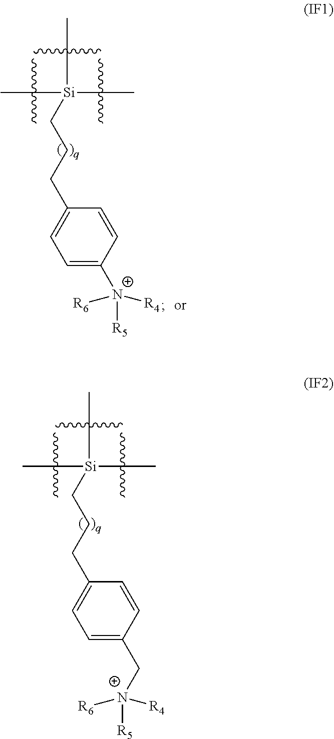

wherein
R₄ and R₅ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof;
R₆ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof;
R is alkyl, aryl, cycloalkyl, heterocycle or any combination thereof;
q is an integer between 0 and 16; and wherein said monomeric unit is chemically bound to the surface of an inorganic core directly or via a third linker (L₆).

In another embodiment, a monomeric unit (as described in e.g., FIGS. 2A-2B and formulas IE and I-VII) within the anti-microbial active unit of this invention is represented by the structure of formula IG1 or IG2.

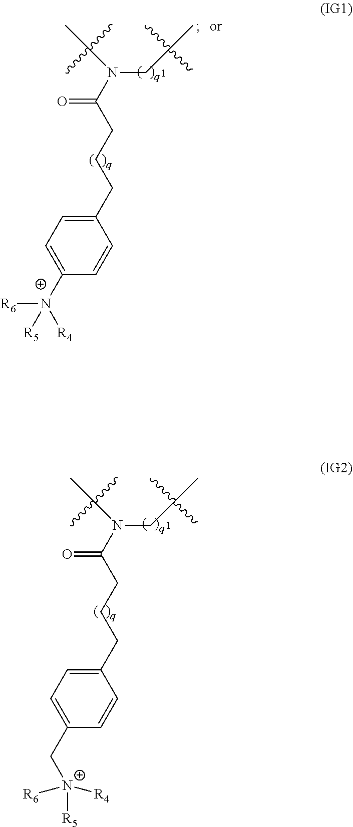

wherein
R₄-R₆ are as described hereinabove;
q and q¹ are independently an integer between 0 and 16; and
wherein said monomeric unit is chemically bound to the surface of an inorganic core directly or via a third linker (L₆).

In another embodiment, a linker molecule which may be used in the processes of preparing the anti-microbial particles of this invention is represented by the structure of formula IH1 or IH2:

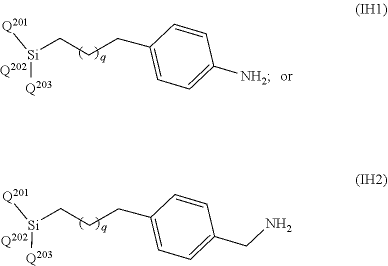

wherein
Q²⁰¹, Q²⁰² and Q²⁰³ are independently selected from the group consisting of alkoxy, methyl, ethyl, hydrogen, sulfonate and halide, wherein at least one of Q²⁰¹, Q²⁰² and Q²⁰³ is selected from ethoxy, methoxy, sulfonate (e.g., mesyl, tosyl) and halide;
q is an integer between 0 and 16;
the linker molecule is capable of being chemically bound to the surface of the inorganic core through the silicon atom; and
the anti-microbial group is introduced by functionalizing the primary amine to obtain an anti-microbial active quaternary ammonium group as described above.

In another embodiment, a linker molecule which may be used in the processes of preparing the anti-microbial particles of this invention is represented by the structure of formula IJ:

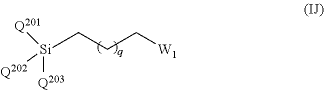

wherein
Q²⁰¹, Q²⁰² and Q²⁰³ are independently selected from the group consisting of alkoxy, methyl, ethyl, hydrogen, sulfonate and halide, wherein at least one of Q²⁰¹, Q²⁰² and Q²⁰³ is selected from ethoxy, methoxy, sulfonate (e.g., mesyl, tosyl) and halide;
W₁ is selected from the group consisting of arylene-NH₂, benzylene-NH₂, halide, sulfonate and hydroxyl;
q is an integer between 0 and 16;
said linker is capable of being chemically bound to the surface of said inorganic core through the silicon atom; and
the anti-microbial group is introduced by substituting the group W with an anti-microbial group, or converting the group W to an anti-microbial group.

### Anti-Microbial Groups Comprising One Long Alkyl Group.

In one embodiment, the anti-microbial group of this invention contains one alkyl group which have from 4 to 24 carbon atoms as R₈-R₁₁ and/or R_{8′}-R_{11′} of the anti-microbial groups

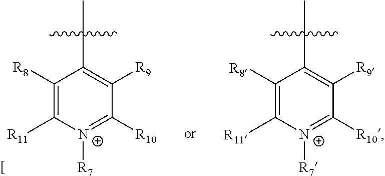

defined in structures (I) and (IE)].

The term “quaternary ammonium group” refers to a group of atoms consisting of a nitrogen atom with four substituents (different than hydrogen) attached thereto. In another embodiment, a “quaternary ammonium group” refers to a group of atoms consisting of a nitrogen atom with four groups wherein each of the group is attached to the nitrogen through a carbon atom. The term “long alkyl group” or chain refers to such an alkyl group or chain which is substituted on the nitrogen atom of the quaternary ammonium group or found as substituent to the pyridinum and which has between 4 and 24 carbon atoms. In some embodiments, the alkyl group is an alkyl group having 4 to 18 carbon atoms. In some embodiments, the alkyl group is an alkyl group having 4 to 8 carbon atoms. In some embodiments, the alkyl group is an alkyl group having 4 to 10 carbon atoms. In other embodiments, the alkyl group is an alkyl group having 6, 7, or 8 carbon atoms, with each possibility representing a separate embodiment of this invention.

### Polymerizable Units

In some embodiment, the polymerizable unit is represented by structure (7):

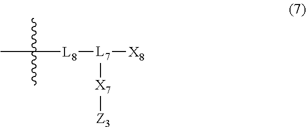

wherein
L₇ is a first linker or a bond;
L₈ is a third linker or a bond;
X₇ is a bond, alkylene, arylene, alkenylene, alkynylene or any combination thereof;
X₈ is nothing or hydrogen;
Z₃ is a functional polymerizable group, or a multiplicity of functional polymerizable groups, connected with a linker in-between, wherein each functional polymerizable group is independently selected from the group consisting essentially of substituted or non-substituted acrylate, substituted or non-substituted epoxy, substituted or non-substituted vinyl or isocyanate moiety, wherein each substituent is alkyl, alkoxy, haloalkyl, halide, cycloalkyl, aryl, heterocycle or C(═O)O(alkyl); and
the notion “ [symbol] ” denotes a covalent bond to an organic or inorganic core.

Non-limiting examples for epoxy groups of Z₃, which may be in certain embodiments substituted, include 3-Glycidyloxypropyl.

Non-limiting examples for vinyl groups of Z₃ include non-substituted or substituted vinyl.

In another embodiment, Z₃ is —O—C(O)—CH═CH₂ (acrylate); —O—C(O)—C(CH₃)=CH₂ (methacrylate);

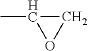

(epoxy); —CH═CH₂ (vinyl) or —N═C═O (isocyanate). In another embodiment, Z₃ is a multiplicity of functional polymerizable groups, connected with a linker in-between. In yet other embodiment, Z₃ is —(CH═CH)₁₋₆—CH═CH₂ (oligoacetylene); or

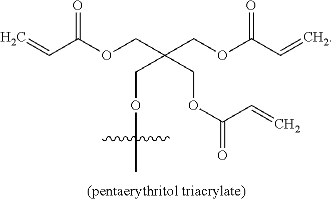 [SMILES: *OCC(COC(=O)C=C)(COC(=O)C=C)COC(=O)C=C]

In another embodiment, the polymerizable unit is Si-alkylene-O—C(O)—CH═CH₂ (Si-acrylate);

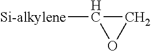

(Si-epoxy); Si-alkylene-CH═CH₂ (Si-vinyl); Si-C H═CH₂ (Si-direct-vinyl), Si-alkylene-N═C═O (Si-isocyanate), {N}-alkylene-O—C(O)—CH═CH₂ (N-acrylate);

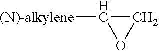

(N-epoxy); {N}-alkylene-CH═CH₂ (N-vinyl), {N}-CH═CH₂ (N-direct-vinyl), or {N}-alkylene-N═C═O (N-isocyanate); wherein the “alkylene” may include etheric oxygen as in e.g. 3-Glycidyloxypropyl (an epoxy moeity); wherein “Si” is a part of siloxane moiety connected to the core directly; and “{N}” is a nitrogenous (comprising nitrogen atom) linker, e.g., amide, amine, guanidine, imidazole etc.

Non-limiting examples for epoxy groups of the polymerizable unit, which may be in certain embodiments substituted, include 3-Glycidyloxypropyl.

In one embodiment, X₇ is a bond, alkylene, arylene, alkenylene, alkynylene or any combination thereof. Each possibility represents a separate embodiment of this invention.

In one embodiment, X₈ is nothing or hydrogen. Each possibility represents a separate embodiment of this invention.

In some embodiments of the polymerizable units, L₇ or L₈ is each independently the same or a different linker. In some embodiments, L₇ and L₈ are connected to each other, in any possible way. In some embodiment, L₈ is nothing (or a bond) and L₇ is connected to the core covalently. In another embodiment, L₈ is connected to the core covalently and L₇ is connected to L₈. In another embodiment, L₇ is connected to X₇, X₈ and L₈ or core. In another embodiment, a “linker” comprises any possible chemical moiety capable of connecting at least two other chemical moieties which are adjacent to such linker. In other embodiments, the linker comprises a functional group. In another embodiment, the linker comprises two (same or different) functional groups. In another embodiment, the functional group comprises phosphate, phosphonate, siloxane, silane, ether acetal, amide, amine, anhydride, ester, ketone, or aromatic ring or rings functionalized with any of the preceding moieties. Each possibility represents a separate embodiment of this invention.

In another embodiment, the monomeric unit of the polymerizable unit comprises a first and/or second linker/s (L₇ or L₈) and a functional polymerizable group. In another embodiment, L₇ and/or L₈ are/is the backbone (they are e.g., alkylene, polypeptide or oligosiloxane (—Si(OH)₂—O— or —Si(CH₃)₂—O—) moieties) of the polymerizable unit. In some embodiments, the linker comprises a functional group. In another embodiment, the linker comprises two (same or different) functional groups. In another embodiment, the functional group comprises phosphate, phosphonate, siloxane, silane, ether, acetal, hydroxyl, amide, amine, anhydride, ester, ketone, or aromatic ring or rings functionalized with any of the preceding moieties. Each possibility represents a separate embodiment of this invention.

In another embodiment, L₇, L₈, X₇, X₈ or any combination thereof is a C1 to C18 alkylene, alkenylene, alkynylene or aryl substituted with at least one carboxyl moiety, wherein the carboxyl end is attached to the core. It may be derived from a C1 to C18 alkylene substituted with at least one carboxyl moiety and having an amino end which is functionalized with e.g., alkylene terminated with epoxy, acrylate, isocyanate or vinyl. This linker may be derived from an amino acid of natural or synthetic source having a chain length of between 2 and 18 carbon atoms (polypeptide), or an acyl halide of said amino acid. Non-limiting examples for such amino acids are 18-amino octadecanoic acid and 18-amino stearic acid.

In another embodiment, L₇, L₈, X₇, X₈ or any combination thereof is a C1 to C18 alkylene, alkenylene, alkynylene, arylene or aryl. This linker may be derived from a di-halo alkylene or di-haloarylene, which is functionalized at each end with the core and functional polymerizable group, respectively, by replacement of the halogen moiety to a functional group that binds to the core and replacement of the halogen moiety to obtain epoxy, acrylate, isocyanate or vinyl moiety.

In another embodiment, L₇, L₈, X₇, X₈ or any combination thereof is an aromatic group derived from non-limiting examples of 4,4-biphenol, dibenzoic acid, dibenzoic halides, dibenzoic sulphonates, terephthalic acid, tetrphthalic halides, and terephthalic sulphonates. This linker is functionalized with the core and functional polymerizable group, respectively, through the functional group of the aromatic group mentioned herein (e.g., hydroxyl, carboxy or sulfonate).

In another embodiment, L₇, L₈, X₇, X₈, or any combination thereof, is a siloxane or silane group derived and/or selected from non-limiting examples of trialkoxyalkylsilane, trialkoxyarylsilane, trihaloalkylsilane, trihaloarylsilane, 3-aminopropyltriethoxysilane (APTES), (3-glycidyloxypropyl)trimethoxysilane and N-2-aminoethyl-3-aminopropyl trimethoxysilane. This linker is functionalized with the core and functional polymerizable group, respectively, through the functional group thereof (e.g., hydroxyl, siloxane, carboxy, amide or sulfonate).

General Definitions for Particles of this Invention

In some embodiments, the anti-microbial unit within the anti-microbial particles of this invention comprises an anti-microbial group comprising a tertiary amine or a tertiary ammonium; or a quaternary ammonium and/or a pyridinium, as represented by the following formulas: —⁺N(R₁)(R₂)(R₃), —⁺N(R₁)(R₂)—, —⁺NH(R₁)(R₂), —⁺NH(R₁)—, —N(R₁)(R₂), —N(R₁)—, —⁺N(R₁′)(R₂′)(R₃′), —⁺N(R₁′)(R₂′)—, —⁺NH(R₁′)(R₂′), —⁺NH(R₁′)—, —N(R₁′)(R₂′), —N(R₁′)—,

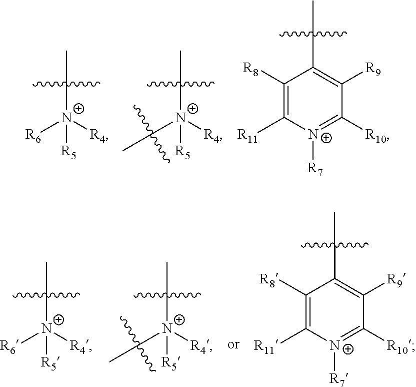

wherein:
R₁-R₁₁ and R₁′-R₁₁′ are as described hereinabove.

In one other embodiment, the quaternary ammonium's, tertiary amine's or tertiary ammonium's or pyridinium's activity remains strong at any pH.

In some embodiments, the polymerizable unit within the antimicrobial particles of this invention is Si-alkylene-O—C(O)—CH═CH₂ (Si-acrylate);

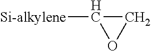

(Si-epoxy); Si-alkylene-CH═CH₂ (Si-vinyl); Si-alkylene-N═C═O (Si-isocyanate), {N}-alkylene-O—C(O)—CH═CH₂ (N-acrylate);

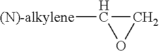

(N-epoxy); {N}-alkylene-CH═CH₂ (N-vinyl) or {N}-alkylene-N═C═O (N-isocyanate); wherein “Si” is apart of siloxane moiety connected to the core directly; and “{N}” is a nitrogenous (comprising nitrogen atom) linker, e.g., amide, amine, guanidine, imidazole etc.

As used herein, the term “alkyl” or “alkylene” refer to any linear- or branched-chain alkyl group containing up to about 24 carbons unless otherwise specified. In one embodiment, an alkyl includes C₁-C₃ carbons. In one embodiment, an alkyl includes C₁-C₄ carbons. In one embodiment, an alkyl includes C₁-C₅ carbons. In another embodiment, an alkyl includes C₁-C₆ carbons. In another embodiment, an alkyl includes C₁-C₈ carbons. In another embodiment, an alkyl includes C₁-C₁₀ carbons. In another embodiment, an alkyl includes C₁-C₁₂ carbons. In another embodiment, an alkyl includes C₄-C₈ carbons. In another embodiment, an alkyl includes C₄-C₁₀ carbons. In another embodiment, an alkyl include C₄-C₁₈ carbons. In another embodiment, an alkyl include C₄-C₂₄ carbons. In another embodiment, an alkyl includes C₁-C₁₈ carbons. In another embodiment, an alkyl includes C₂-C₁₈ carbons. In another embodiment, branched alkyl is an alkyl substituted by alkyl side chains of 1 to 5 carbons. In one embodiment, the alkyl group may be unsubstituted. In another embodiment, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the alkyl is a 2,2-disubstituted C₃-C₂₀ alkyl. The term “2,2-disubstituted C₃-C₂₀ alkyl” refers to alkyl as described herein, having between 3 and 20 carbons and is substituted thrice at the second carbon (from the connection point) with halogen, haloalkyl, alkyl, alkoxy, carbonyl, amido, alkylamido, dialkylamido, cyano, nitro, CO₂H, amino, alkylamino, dialkylamino, carboxyl, thio and/or thioalkyl, where such substitutions can be the same or different; or alternatively it is substituted once at the second carbon with oxo (=O) or with other double bond to an element (e.g., S) or a moiety (e.g., vinylic carbon or NH) and it's further substituted with a substitutent selected from the above list of the first possibility; in all cases—no hydrogen is available for abstraction at this second carbon position (e.g., no hydrogens are found at this position, only non-hydrogen substituents). Non-limiting examples of 2,2-disubstituted C₃-C₂₀ alkyl include neopentyl (—CH₂—C(CH₃)₃, —CH₂—C(CH₃)₂—CH₂CH₃, CH₂—CF₂CH₃ and —CH₂C(═O)CH₃. In another embodiment, the alkyl is a 2,2-disubstituted C₃-C₈ alkyl. In another embodiment, the alkyl is a 2,2-disubstituted C₃-C₁₀ alkyl. In another embodiment, the alkyl is a 2,2-disubstituted C₃-C₁₂ alkyl. In another embodiment, the alkyl is a 2,2-disubstituted C₃-C₁₈ alkyl. The terms “2,2-disubstituted C₃-C₈ alkyl”, “2,2-disubstituted C₃-C₁₀ alkyl”, “2,2-disubstituted C₃-C₁₂ alkyl” and “2,2-disubstituted C₃-C₁₈ alkyl” refer to similar moiety as “2,2-disubstituted C₃-C₂₀ alkyl” but with C₃-C₈, C₃-C₁₀, C₃-C₁₂ and C₃-C₁₈ alkyl, respectively. In another embodiment, alkylene is a 2,2-disubstituted C₂-C₂₀ alkylene. The term “2,2-disubstituted C₂-C₂₀ alkylene” refers to similar moiety as “2,2-disubstituted C₃-C₂₀ alkyl” but with alkylene as described herein which has between 2 and 20 carbons. Non-limiting examples of 2,2-disubstituted C₂-C₂₀ alkylene include neopentylene (—CH₂—C(CH₃)₂—CH₂—, —CH₂—C(CH₃)₂—CH₂CH₂—, —CH₂—CF₂CH₂— and —CH₂C(═O)CH₂—. In another embodiment, the alkylene is a 2,2-disubstituted C₂-C₈ alkylene. In another embodiment, the alkylene is a 2,2-disubstituted C₂-C₁₀ alkylene. In another embodiment, the alkylene is a 2,2-disubstituted C₂-C₁₂ alkylene. In another embodiment, the alkylene is a 2,2-disubstituted C₂-C₁₈ alkylene. The terms “2,2-disubstituted C₂-C₈ alkylene”, “2,2-disubstituted C₂-C₁₀ alkylene”, “2,2-disubstituted C₂-C₁₂ alkylene” and “2,2-disubstituted C₂-C₁₈ alkylene” refer to similar moiety as “2,2-disubstituted C₂-C₂₀ alkylene” but with C₂-C₈, C₂-C₁₀, C₂-C₁₂ and C₂-C₁₈ alkylene, respectively.

In another embodiment, the alkyl is a 2,2,2-trisubstituted ethyl. The term “2,2,2-trisubstituted ethyl” refers to ethyl substituted thrice at the second carbon (from the connection point) with halogen, haloalkyl, alkoxy, carbonyl, amido, alkylamido, dialkylamido, cyano, nitro, CO₂H, amino, alkylamino, dialkylamino, carboxyl, thio and/or thioalkyl, where such substitutions can be the same or different; or alternatively it is substituted once at the second carbon with oxo (=O) or with other double bond to an element (e.g., S) or a moiety (e.g., vinylic carbon or NH) and it's further substituted with a substituent selected from the above list of the first possibility; in all cases—no hydrogen is available for abstraction at this second carbon position (e.g., no hydrogens are found at this position, only non-hydrogen substituents). Non-limiting examples of 2,2,2-trisubstituted ethyl include 2,2,2 trihaloethyl and —CH₂C(═O)—NH₂. In another embodiment hydrophobic alkyl refers to an alkyl having at least four carbons. In another embodiment hydrophobic alkyl refers to a C₄-C₂₄ alkyl. In another embodiment hydrophobic alkyl refers to a C₄-C₈ alkyl. In another embodiment hydrophobic alkyl refers to a C₄ alkyl. In another embodiment hydrophobic alkyl refers to a C₅ alkyl. In another embodiment hydrophobic alkyl refers to a C₆ alkyl. In another embodiment hydrophobic alkyl refers to a C₇ alkyl. In another embodiment hydrophobic alkyl refers to a C₈ alkyl.

As used herein, the term “aryl” refers to any aromatic ring that is directly bonded to another group and can be either substituted or unsubstituted. As used herein, the term “Arylene” refers to the same where it is directly bonded to two groups (e.g., arylene is e.g., phenylene, —C₆H₄—). In another embodiment, it can be directly bonded to more than two groups. The aryl or arylene group can be a sole substituent, or it can be a component of a larger substituent, such as in an arylalkyl, arylamino, arylamido, etc. Exemplary aryl (and similarly, arylene) groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc. Substitutions include but are not limited to: F, Cl, Br, I, C₁-C₅ linear or branched alkyl, C₁-C₅ linear or branched haloalkyl, C₁-C₅ linear or branched alkyl or alkoxy, C₁-C₅ linear or branched haloalkyl or haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂. In another embodiment, hydrophobic aryl or arylene refers to aryl or arylene having at least six carbons.

As used herein, the term “benzyl” refers to the —CH₂—C₆H₅ moiety and can be unsubstituted or substituted with the following non-limiting list of substituents: F, Cl, Br, I, C₁-C₅ linear or branched alkyl, C₁-C₅ linear or branched haloalkyl, C₁-C₅ linear or branched alkyl or alkoxy, C₁-C₅ linear or branched haloalkyl or haloalkoxy, CF₃, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, or —C(O)NH₂. Similarly, “benzylene” refers to the —CH₂—C₆H₄— moiety and can be unsubstituted or substituted with the substituents described above for the benzyl moiety.

As used herein, the term “haloalkyl” refers to alkyl as described hereinabove and substituted at least once by halide (e.g., F, Cl, Br or I). In one embodiment, all of the alkyl is substituted by halides, e.g., no hydrogens are found in the haloalkyl, and is termed “perhaloalkyl” (e.g., CF₃: perfluoromethyl or CCl₃: perchloromethyl). In one embodiment, only part of the alkyl is substituted by halides (e.g., CH₂CF₃). In another embodiment, non limiting examples of haloalkyls include: CF₃, CCl₃, CH₂CF₃, CF₂CF₃, CCl₂CCl₃ and CI₃.

The term “alkenyl” or “alkenylene” refer to a substance that includes at least two carbon atoms and at least one double bond. The term “1-alkenyl” or “1-alkenylene” refers to the same, where the double bond is on the first carbon (from the connection point). The term “2-alkenyl” or “2-alkenylene” refers to the same, where the double bond is on the second carbon (from the connection point). The term “3-alkenyl” or “3-alkenylene” refers to the same, where the double bond is on the third carbon (from the connection point). In one embodiment, the alkenyl has 2-7 carbon atoms. In another embodiment, the alkenyl has 2-12 carbon atoms. In another embodiment, the alkenyl has 2-10 carbon atoms. In another embodiment, the alkenyl has 3-6 carbon atoms. In another embodiment, the alkenyl has 2-4 carbon atoms. In another embodiment, the alkenyl has 4-8 carbon atoms. In another embodiment hydrophobic alkenyl refers to alkenyl having at least four carbons. In another embodiment hydrophobic alkenyl refers to a C₄-C₈ alkenyl.

The term “alkynyl” or “alkynylene” refers to a substance that includes at least two carbon atoms and at least one triple bond. The term “1-alkynyl” or “1-alkynylene” refers to the same, where the triple bond is on the first carbon (from the connection point). The term “2-alkynyl” or “2-alkynylene” refers to the same, where the triple bond is on the second carbon (from the connection point). The term “3-alkynyl” or “3-alkynylene” refers to the same, where the triple bond is on the third carbon (from the connection point). In one embodiment, the alkynyl has 2-7 carbon atoms. In another embodiment, the alkynyl has 2-12 carbon atoms. In another embodiment, the alkynyl has 2-10 carbon atoms. In another embodiment, the alkynyl has 3-6 carbon atoms. In another embodiment, the alkynyl has 2-4 carbon atoms. In another embodiment, the alkynyl has 3-6 carbon atoms. In another embodiment, the alkynyl has 4-8 carbon atoms. In another embodiment hydrophobic alkynyl refers to alkynyl having at least four carbons. In another embodiment hydrophobic alkynyl refers to a C₄-C₈ alkenyl.

The term “alkoxy” refers in one embodiment to an alky as defined above bonded to an oxygen. Non limiting examples of alkoxy groups include: methoxy, ethoxy and isopropoxy.

A “cycloalkyl” group refers, in one embodiment, to a ring structure comprising carbon atoms as ring atoms, which may be either saturated or unsaturated, substituted or unsubstituted; and is directly bonded to one group (e.g., cyclohexyl-, C₆H₁₁—). In another embodiment the cycloalkyl is a 3-12 membered ring. In another embodiment the cycloalkyl is a 6 membered ring. In another embodiment the cycloalkyl is a 5-7 membered ring. In another embodiment the cycloalkyl is a 3-8 membered ring. In another embodiment, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the cycloalkyl ring may be fused to another saturated or unsaturated cycloalkyl or heterocyclic 3-8 membered ring. In another embodiment, the cycloalkyl ring is a saturated ring. In another embodiment, the cycloalkyl ring is an unsaturated ring. Non-limiting examples of a cycloalkyl group comprise cyclohexyl, cyclohexenyl, cyclopropyl, cyclopropenyl, cyclopentyl, cyclopentenyl, cyclobutyl, cyclobutenyl, cycloctyl, cycloctadienyl (COD), cycloctaene (COE) etc. In another embodiment hydrophobic cycloalkyl refers to a cycloalkyl having at least six carbons. A “cycloalkylene” group refers, in one embodiment, to the same definitions above of “cycloalkyl”, however the cycloalkylene is directly bonded to two groups (e.g., -cyclohexylene-, —C₆H₁₀—). In another embodiment, it is directly bonded to more than two groups.

A “heterocycle” group refers, in one embodiment, to a ring structure comprising in addition to carbon atoms, sulfur, oxygen, nitrogen or any combination thereof, as part of the ring. In another embodiment the heterocycle is a 3-12 membered ring. In another embodiment the heterocycle is a 6 membered ring. In another embodiment the heterocycle is a 5-7 membered ring. In another embodiment the heterocycle is a 3-8 membered ring. In another embodiment, the heterocycle group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio and/or thioalkyl. In another embodiment, the heterocycle ring may be fused to another saturated or unsaturated cycloalkyl or heterocyclic 3-8 membered ring. In another embodiment, the heterocyclic ring is a saturated ring. In another embodiment, the heterocyclic ring is an unsaturated ring. Non limiting examples of a heterocyclic rings comprise pyridine, piperidine, morpholine, piperazine, thiophene, pyrrole, benzodioxole, or indole. In another embodiment hydrophobic heterocyclic group refers to a heterocycle having at least six carbons. In one embodiment, the heterocycle is directly bonded to one group (e.g., pyridinyl,

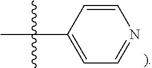 [SMILES: *c1ccncc1]

In one embodiment, the heterocycle is directly bonded to two groups (e.g., pyridinylene,

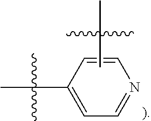

In one embodiment, the heterocycle is directly bonded to more than two groups.

The term “hydrophobic” refers to an alkyl, alkenyl or alkynyl having at least four carbons, or the term hydrophobic refers to terpenoid, to cycloalkyl, aryl or heterocycle having at least six carbons. Each possibility represents a separate embodiment of this invention.

The anti-microbial groups of this invention (e.g., of all anti-microbial active units, sections “A)”-“C)” above) are chemically bound to the core at a surface density of at least one anti-microbial group per 10 sq. nm of the core surface. In another embodiment at least 1 anti-microbial group per 1 sq nm of the core surface. In another embodiment between 0.001-300 anti-microbial groups per sq nm of the core surface. In another embodiment between 0.001-250 anti-microbial groups per sq nm of the core surface. In another embodiment between 0.001-200 anti-microbial groups per sq nm of the core surface. In another embodiment between 0.001-150 anti-microbial groups per sq nm of the core surface. In another embodiment between 0.001-100 anti-microbial groups per sq nm of the core surface. In another embodiment between 0.001-50 anti-microbial groups per sq nm of the core surface. In another embodiment between 0.001-20 anti-microbial groups per sq nm of the core surface. In another embodiment between 0.001-17 anti-microbial groups per sq nm of the core surface. In another embodiment between 0.001-15 anti-microbial groups per sq nm of the core surface. In another embodiment between 0.001-10 anti-microbial groups per sq nm of the core surface. In another embodiment between 0.001-4 anti-microbial groups per sq nm of the core surface. In another embodiment between 0.001-1 anti-microbial groups per sq nm of the core surface. In another embodiment between 50-100 anti-microbial groups per sq nm of the core surface. In another embodiment between 100-150 anti-microbial groups per sq nm of the core surface. In another embodiment between 150-200 anti-microbial groups per sq nm of the core surface. In another embodiment between 200-250 anti-microbial groups per sq nm of the core surface. In another embodiment between 250-300 anti-microbial groups per sq nm of the core surface. In another embodiment between 1-4 anti-microbial groups per sq nm of the core surface. In another embodiment between 1-6 anti-microbial groups per sq nm of the core surface. In another embodiment between 1-20 anti-microbial groups per sq nm of the core surface. In another embodiment between 1-10 anti-microbial groups per sq nm of the core surface. In another embodiment between 1-15 anti-microbial groups per sq nm of the core surface. Each possibility represents a separate embodiment of this invention.

In some embodiments, the number of the anti-microbial groups [(n₁+n₂)×m] per each anti-microbial active unit (e.g., as represented sections “A)”-“C)” above) is between 1-200. In another embodiment, the number of the anti-microbial groups per each anti-microbial active unit is between 1-150. In another embodiment, the number of the anti-microbial groups per each anti-microbial active unit is between 1-100. In another embodiment, the number of the anti-microbial groups per each anti-microbial active unit is between 1-50. In another embodiment, the number of the anti-microbial groups per each anti-microbial active unit is between 1-30. In another embodiment, the number of the anti-microbial groups per each anti-microbial active unit is between 1-20. In another embodiment, the number of the anti-microbial groups per each anti-microbial active unit is between 1-10. In another embodiment, the number of the anti-microbial groups per each anti-microbial active unit is between 50-100. In another embodiment, the number of the anti-microbial groups per each anti-microbial active unit is between 100-150. In another embodiment, the number of the anti-microbial active unit per each anti-microbial active unit is between 150-200. Each possibility represents a separate embodiment of this invention.

In some embodiments, the number of the monomeric units per each anti-microbial active unit (e.g., as represented sections “A)”-“C)” above) is between 1-200. In another embodiment, the number of the monomeric units per each anti-microbial active unit is between 1-150. In another embodiment, the number of the monomeric units per each anti-microbial active unit is between 1-100. In another embodiment, the number of the monomeric units per each anti-microbial active unit is between 1-50. In another embodiment, the number of the monomeric units per each anti-microbial active unit is between 1-30. In another embodiment, the number of monomeric units per each anti-microbial active unit is between 1-20. In another embodiment, the number of the monomeric units per each anti-microbial active unit is between 1-10. In another embodiment, the number of the monomeric units per each anti-microbial active unit is between 50-100. In another embodiment, the number of the monomeric units per each anti-microbial active unit is between 100-150. In another embodiment, the number of the monomeric units per each anti-microbial active unit is between 150-200. Each possibility represents a separate embodiment of this invention.

In one embodiment, n₁ is between 0-200. In another embodiment, n₁ is between 0-10. In another embodiment, n₁ is between 10-20. In another embodiment, n₁ is between 20-30. In another embodiment, n₁ is between 30-40. In another embodiment, n₁ is between 40-50. In another embodiment, n₁ is between 50-60. In another embodiment, n₁ is between 60-70. In another embodiment, n₁ is between 70-80. In another embodiment, n₁ is between 80-90. In another embodiment, n₁ is between 90-100. In another embodiment, n₁ is between 100-110. In another embodiment, n₁ is between 110-120. In another embodiment, n₁ is between 120-130. In another embodiment, n₁ is between 130-140. In another embodiment, n₁ is between 140-150. In another embodiment, n₁ is between 150-160. In another embodiment, n₁ is between 160-170. In another embodiment, n₁ is between 170-180. In another embodiment, n₁ is between 180-190. In another embodiment, n₁ is between 190-200. Each possibility represents a separate embodiment of this invention.

In one embodiment, n₂ is between 0-200. In another embodiment, n₂ is between 0-10. In another embodiment, n₂ is between 10-20. In another embodiment, n₂ is between 20-30. In another embodiment, n₂ is between 30-40. In another embodiment, n₂ is between 40-50. In another embodiment, n₂ is between 50-60. In another embodiment, n₂ is between 60-70. In another embodiment, n₂ is between 70-80. In another embodiment, n₂ is between 80-90. In another embodiment, n₂ is between 90-100. In another embodiment, n₂ is between 100-110. In another embodiment, n₂ is between 110-120. In another embodiment, n₂ is between 120-130. In another embodiment, n₂ is between 130-140. In another embodiment, n₂ is between 140-150. In another embodiment, n₂ is between 150-160. In another embodiment, n₂ is between 160-170. In another embodiment, n₂ is between 170-180. In another embodiment, n₂ is between 180-190. In another embodiment, n₂ is between 190-200. Each possibility represents a separate embodiment of this invention.

In one embodiment, m is between 1-200. In another embodiment, m is between 1-10. In another embodiment, m is between 10-20. In another embodiment, m is between 20-30. In another embodiment, m is between 30-40. In another embodiment, m is between 40-50. In another embodiment, m is between 50-60. In another embodiment, m is between 60-70. In another embodiment, m is between 70-80. In another embodiment, m is between 80-90. In another embodiment, m is between 90-100. In another embodiment, m is between 100-110. In another embodiment, m is between 110-120. In another embodiment, m is between 120-130. In another embodiment, m is between 130-140. In another embodiment, m is between 140-150. In another embodiment, m is between 150-160. In another embodiment, m is between 160-170. In another embodiment, m is between 170-180. In another embodiment, m is between 180-190. In another embodiment, m is between 190-200. Each possibility represents a separate embodiment of this invention.

In another embodiment, the anti-microbial group of this invention may be selected from: (a) a quaternary ammonium group comprising at least one terpenoid moiety or one hydrophobic group; and (b) a pyridinium group. Each possibility represents a separate embodiment of this invention.

The term “nanoparticle” as used herein refers to a particle having a diameter of less than about 1,000 nm. The term “microparticle” as used herein refers to a particle having a diameter of about 1,000 nm or larger.

The anti-microbial particles of this invention are characterized by having a diameter between about 5 to about 100,000 nm, and thus encompass both nanoparticulate and microparticulate compositions. For example, the particles may be between about 10 to about 50,000 nm. In other embodiments, the particles are more than 1,000 nm in diameter. In other embodiments, the particles are more than 10,000 nm in diameter. In other embodiment, the particles are between 1,000 and 50,000 nm in diameter. In other embodiment, the particles are between 5 and 250 nm in diameter. In other embodiment, the particles are between 5 and 500 nm in diameter. In another embodiment, the particles are between 5 and 1000 nm in diameter. It is apparent to a person of skill in the art that other particles size ranges are applicable and are encompassed within the scope of this invention.

The anti-microbial group of this invention may be in the form of a quaternary ammonium or pyridinium salt, as described hereinabove. Since any such groups are positively charged, their charge is balanced with an anion. Non-limiting examples of anions include: a halide, e.g., fluoride, chloride, bromide or iodide and fluoride, bicarbonate, nitrate, phosphate, acetate, fumarate, succinate, mesylate, triflate, tosylate, tetrafluoroborate, hexafluorophosphate and sulfate. Each possibility represents a separate embodiment of this invention.

In some embodiments, the term “quaternary ammonium group” refers to a group of atoms consisting of a nitrogen atom with four substituents (different than hydrogen) attached thereto. In another embodiment, a “quaternary ammonium group” refers to a group of atoms consisting of a nitrogen atom with four groups wherein each of the group is attached to the nitrogen through a carbon atom. In some embodiments the quaternary ammonium group provided herein comprises at least one long alkyl chain. In some embodiments the quaternary ammonium group provided herein comprises at least one terpenoid. The term “long alkyl group” or chain refers to such an alkyl group or chain which is substituted on the nitrogen atom of the quaternary ammonium group and which has between 4 and 24 carbon atoms. In some embodiments, the alkyl group is an alkyl group having 4 to 18 carbon atoms. In some embodiments, the alkyl group is an alkyl group having 4 to 8 carbon atoms. In some embodiments, the alkyl group is an alkyl group having 4 to 10 carbon atoms. In other embodiments, the alkyl group is an alkyl group having 6, 7, or 8 carbon atoms, with each possibility representing a separate embodiment of this invention.

### Anti-Microbial Groups Comprising Terpenoid Groups

In one embodiment, the anti-microbial group of this invention, as described hereinabove in regards to the anti-microbial particles and the anti-microbial active units thereof (e.g., Formulae (1)-(6), (I)-(VII), (IA)-(IE), (IF1)-(IF2), (IG1)-(IG2), (I1)-(IH2) and IJ) contains at least one terpenoid group. In another embodiment, the anti-microbial group is selected from: (a) a tertiary amine (R₃ and/or R₃′ is nothing) or tertiary ammonium (R₃ and/or R₃′ is H) comprising at least one terpenoid moiety; and (b) a quaternary ammonium group comprising at least one terpenoid moiety. In another embodiment, when the anti-microbial group of this invention contains at least one terpenoid group and/or R₁, R₂, R₃ and/or R₁′, R₂′, R₃′ of the anti-microbial groups as defined hereinabove are terpenoid moieties—the core of the particles of this invention is a polyhedral oligomeric silsesquioxane (POSS).

In some embodiments, the anti-microbial group of formula (1) to (6) is selected from: (a) a tertiary amine (R₃ and/or R₃′ is nothing) or tertiary ammonium (R₃ and/or R₃′ is H), wherein the nitrogen atom of each tertiary amine/ammonium having at least one bond to X₁ or X₂ and one bond to a terpenoid moiety; (b) a tertiary amine (R₃ and/or R₃′ is nothing), or tertiary ammonium (R₃ and/or R₃′ is H), the nitrogen atom of each tertiary amine/ammonium having one bond to X₁ or X₂ and two bonds to terpenoid moieties which may be the same or different from each other, or a salt of said tertiary amine; (c) a quaternary ammonium group the nitrogen atom of each quaternary ammonium group having at least one bond to X₁ or X₂ and one or two bonds to terpenoid moieties which may be the same or different from each other; Each possibility represents a separate embodiment of this invention.

In one embodiment, R₆, R₈-R₁₁, R_{6′} and/or R_{8′}-R_{11′} of the anti-microbial groups [—⁺N(R₄)(R₅)(R₆), —⁺N(R₄′)(R₅′)(R₆′),

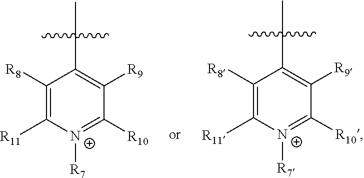

defined in structures (I) and (IE)] are the terpenoid moieties.

In another embodiment, when the anti-microbial group of this invention contains at least one terpenoid group and/or R₆, R₈-R₁₁, R₆, and/or R₈′-R₁₁′ of the anti-microbial groups as defined hereinabove are terpenoid moieties—the core of the particles of this invention is a polyhedral oligomeric silsesquioxane (POSS).

The term “terpenoid”, also known as “isoprenoid” refers to a large class of naturally occurring compounds that are derived from five-carbon isoprene units. A “terpenoid moiety” is derived from a terpenoid.

In some embodiments, the terpenoid moiety is a “terpenoidyl”, e.g., directly bonded to one group (e.g., cinnamyl:

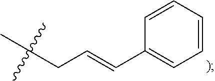

or a “terpenoidylene”, e.g., directly bonded to two groups (e.g., cinnamylene, e.g.,

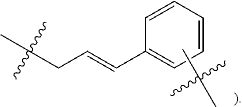

In one embodiment, the terpenoid moiety is directly bonded to more than two groups. In one embodiment, the terpenoid moiety is a cinammyl or cinnamylene group derived from cinnamaldehyde, cinnamic acid, curcumin, viscidone or cinnamyl alcohol. In another embodiment, the terpenoid moiety is a bornyl or a bornylene group derived from camphor, bornyl halide or bornyl alcohol. In another embodiment, the terpenoid moiety is derived from citral. In another embodiment, the terpenoid moiety is derived from perilaldehyde. Each possibility represents a separate embodiment of this invention.

Cinnamaldehyde is a natural aldehyde extracted from the genus Cinnamomum. It is known for its low toxicity and its effectiveness against various bacteria and fungi.

Camphor is found in the wood of the camphor laurel (Cinnamomum camphora), and also of the kapur tree. It also occurs in some other related trees in the laurel family, for example Ocotea usambarensis, as well as other natural sources. Camphor can also be synthetically produced from oil of turpentine. Camphor can be found as an R or S enantiomer, a mixture of enantiomers and a racemic mixture. Each possibility represents a separate embodiment of this invention.

Citral, or 3,7-dimethyl-2,6-octadienal or lemonal, is a mixture of two diastereomeric terpenoids. The two compounds are double bond isomers. The E-isomer is known as geranial or citral A. The Z-isomer is known as neral or citral B. Citral is known to have anti-microbial activity.

Perillaldehyde, also known as perilla aldehyde, is a natural terpenoid found most in the annual herb perilla, as well as in a wide variety of other plants and essential oils.

Other examples of terpenoids include, but are not limited to: curcuminoids found in turmeric and mustard seed, citronellal found in Cymbopogon (lemon grass) and carvacrol, found in Origanum vulgare (oregano), thyme, pepperwort, wild bergamot and Lippia graveolens. Each possibility represents a separate embodiment of this invention.

In accordance with the above embodiment, the anti-microbial active terpenoid moieties are selected from the group consisting of:

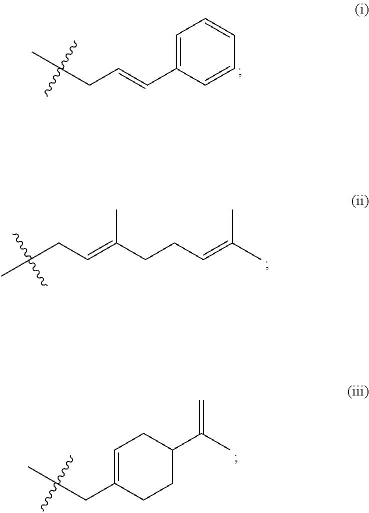 [SMILES: *C/C=C/c1ccccc1 ; *C/C=C(\C)CCC=C(C)C ; *CC1=CCC(C(=C)C)CC1]

or any combination thereof,

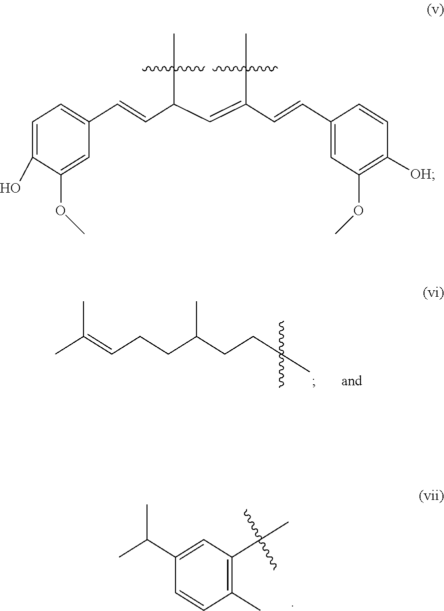 [SMILES: *C(/C=C/c1ccc(O)c(OC)c1)=C\C(*)/C=C/c1ccc(O)c(OC)c1 ; *CCC(C)CCC=C(C)C ; *c1cc(C(C)C)ccc1C]

Each possibility represents a separate embodiment of this invention.

Non-limiting examples of anti-microbial active quaternary ammonium groups in accordance with the principles of this invention are:

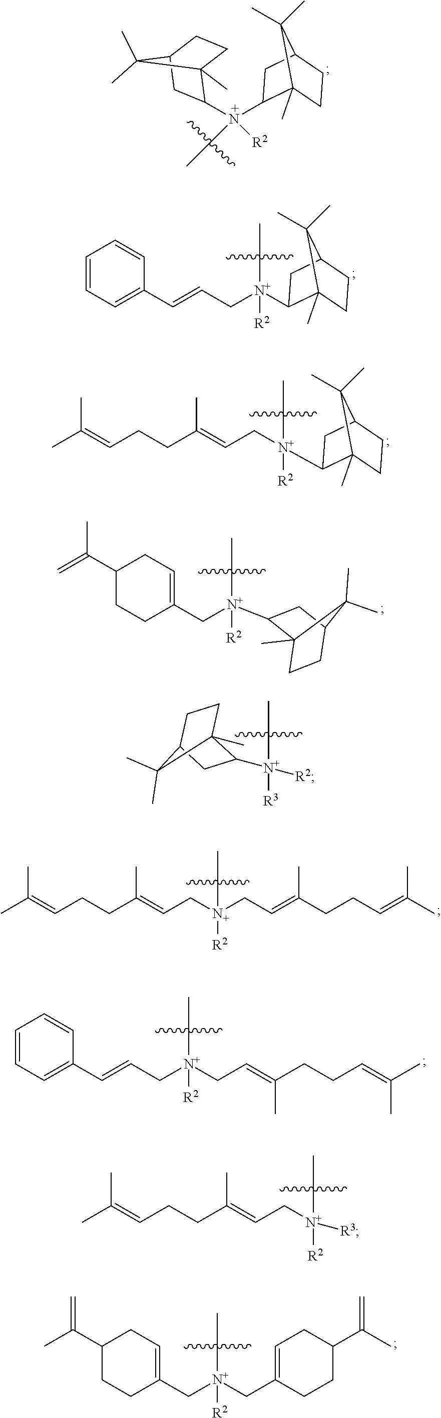

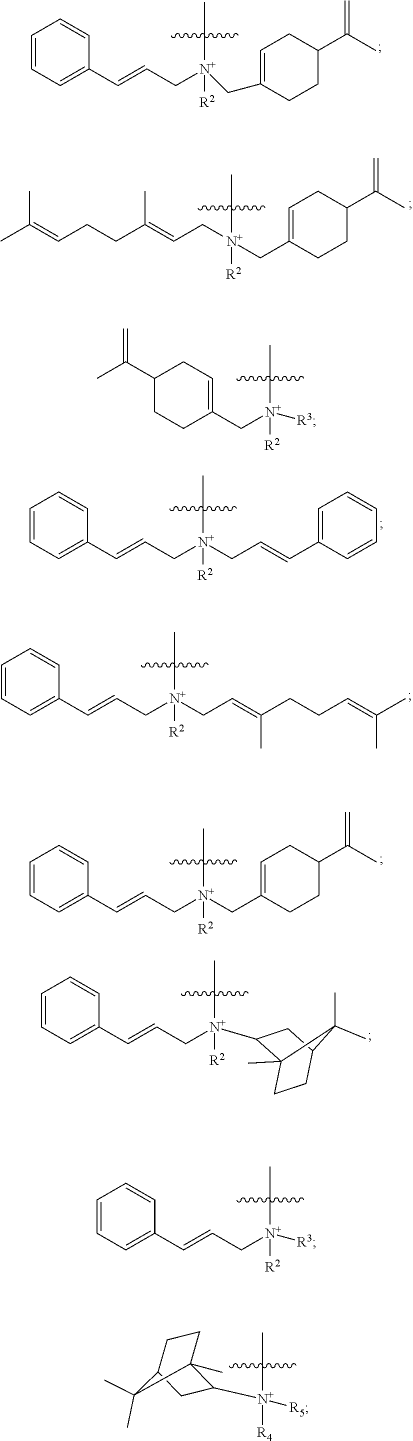

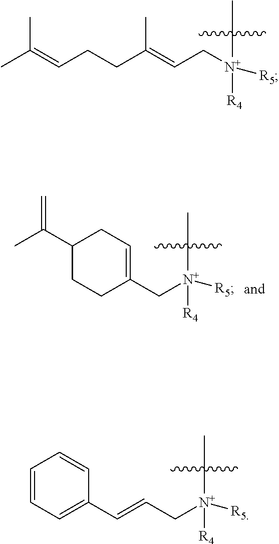

wherein
R² is alkyl, terpenoid, cycloalkyl, aryl, heterocycle, alkenyl, alkynyl or any combination thereof;
R₃ is alkyl, terpenoid moiety, cycloalkyl, aryl, heterocycle, alkenyl, alkynyl or any combination thereof,
R₄ and R₅ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, and
R is alkyl, aryl, cycloalkyl, heterocycle or any combination thereof

Non-limiting examples of functional anti-microbial active tertiary amine groups or its protonated form in accordance with the principles of this invention are:

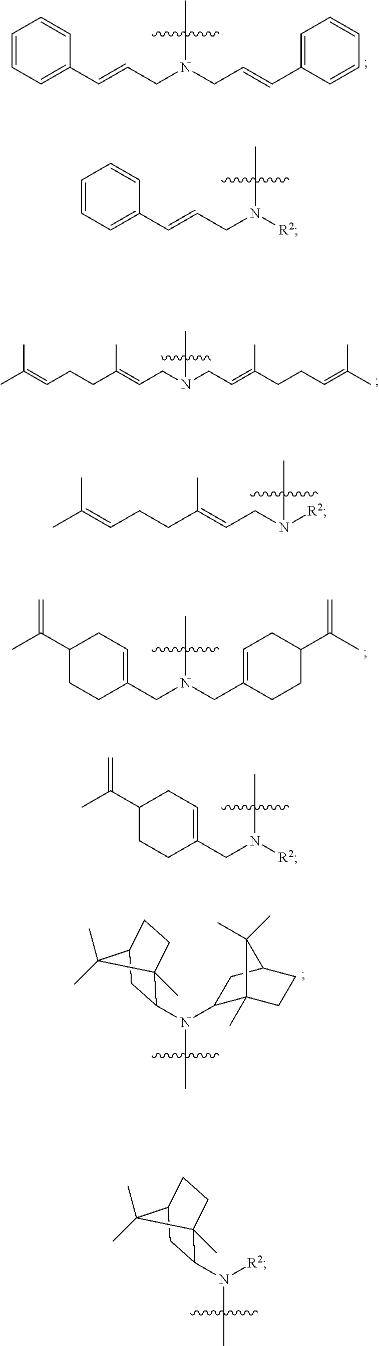

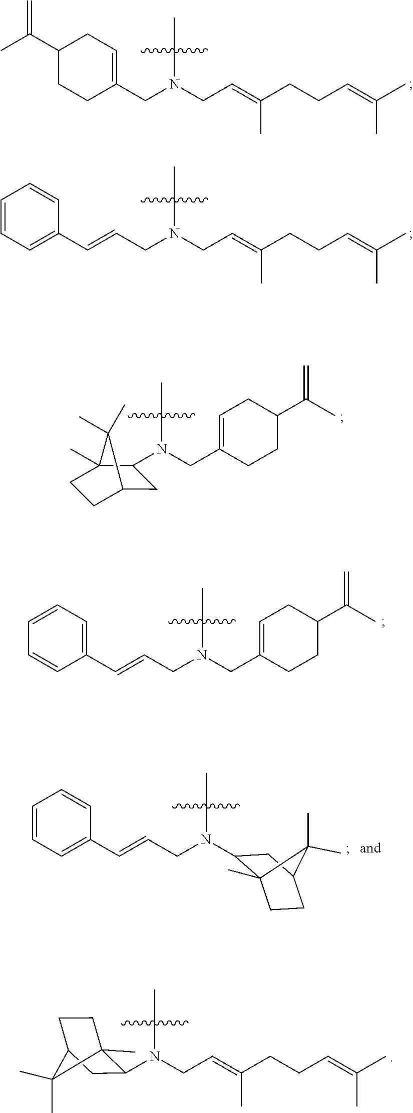

wherein R₂ is alkyl, terpenoid, cycloalkyl, aryl, heterocycle, alkenyl, alkynyl or any combination thereof.

### Inorganic Cores

In one embodiment, the inorganic core of the particles of this invention comprises silica, metal, metal oxide or a zeolite. Each possibility represents a separate embodiment of this invention.

In one embodiment, the core of the particles of this invention comprises silica (SiO₂). The silica may be in any form known in the art, non-limiting examples of which include polyhedral oligomeric silsesquioxane (POSS), amorphous silica, dense silica, aerogel silica, porous silica, mesoporous silica and fumed silica. Each possibility represents a separate embodiment of this invention.

The surface density of active groups onto the particle surface may have proportional impact on its anti-microbial activity. In another embodiment, the core of the particles of this invention comprises glasses or ceramics of silicate (SiO₄⁻⁴). Non-limiting examples of silicates include aluminosilicate, borosilicate, barium silicate, barium borosilicate and strontium borosilicate. Each possibility represents a separate embodiment of this invention.

In another embodiment, the core of the particles of this invention comprises surface activated metals selected from the group of: silver, gold, platinum, palladium, copper, zinc and iron. Each possibility represents a separate embodiment of this invention.

In another embodiment, the core of the particles of this invention comprises metal oxides selected from the group of: zirconium dioxide, titanium dioxide, vanadium dioxide, zinc oxide, copper oxide and magnetite. Each possibility represents a separate embodiment of this invention.

In one embodiment, the inorganic core is polyhedral oligomeric silsesquioxane (POSS) core.

In some embodiments, provided herein is an anti-microbial particle, comprising a POSS core, wherein each silicon atom of the POSS is functionalized by at least one anti-microbial group or by at least one functional polymerizable group, wherein the molar ratio of the silicon atom functionalized by at least one anti-microbial group and the silicon atom functionalized by at least one functional polymerizable group is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility represents a separate embodiment of this invention. In one embodiment, the anti-microbial group is a quaternary ammonium. In one embodiment, anti-microbial group is a tertiary amine. In one embodiment, the anti-microbial group is a tertiary ammonium. In one embodiment, the anti-microbial group comprises the following formulas: —⁺N(R₁)(R₂)(R₃), —⁺N(R₁)(R₂)-, —⁺NH(R₁)(R₂), —⁺NH(R₁)-, —N(R₁)(R₂), —N(R₁)-, —⁺N(R₁′)(R₂′)(R₃′), —⁺N(R₁′)(R₂′)-, —⁺NH(R₁′)(R₂′), —⁺NH(R₁′)-, —N(R₁′)(R₂′) or —N(R₁′)—; wherein R₁-R₃ and R₁′-R₃′ are as described hereinabove. In one embodiment, the anti-microbial group comprises a quaternary ammonium and/or a pyridinium, as represented by the following formulas:

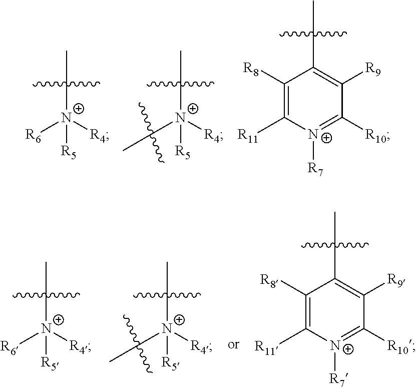

wherein:
R₄-R₁₁ and R₄′-R₁₁′ are as described hereinabove. In another embodiment, the anti-microbial group may be selected from: (a) a quaternary ammonium group comprising at least one terpenoid moiety or one hydrophobic group; and (b) a pyridinium group. Each possibility represents a separate embodiment of this invention.

In one embodiment, the functional polymerizable group comprises an acrylate, an epoxy, a vinyl and/or an isocyanate group.

In some embodiments, provided herein is an anti-microbial particle, comprising a polyhedral oligomeric silsesquioxane (POSS) core wherein each silicon atom of the POSS is functionalized by at least one quaternary ammonium group or by at least one functional polymerizable group wherein the functional polymerizable group comprises an acrylate, an epoxy, an isocyanate or a vinyl group; wherein the number of quaternary ammonium groups per silicon atom is between 1 to 200 and wherein the molar ratio of the silicon atom functionalized by at least one quaternary ammonium and the silicon atom functionalized by at least one functional polymerizable group is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility, as well as subranges thereof, represents a separate embodiment of this invention.

In some embodiments, provided herein is an anti-microbial particle, comprising a polyhedral oligomeric silsesquioxane (POSS) core wherein each silicon atom of the POSS is functionalized by at least one quaternary ammonium group or by at least one functional polymerizable group comprises an acrylate, an epoxy, an isocyanate or a vinyl group; wherein the number of quaternary ammonium groups per silicon atom is between 2 to 200 and wherein the molar ratio of the silicon atom functionalized by at least one quaternary ammonium and the silicon atom functionalized at least one functional polymerizable group is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility, as well as subranges thereof, represents a separate embodiment of this invention.

In some embodiments, the silicon atom of the POSS (core of the anti-microbial particle) is functionalized by at least one quaternary ammonium group. “A silicon atom of the POSS functionalized by at least one quaternary ammonium group” refers to a silicon atom of the POSS (core) which is linked to one or more quaternary ammonium groups by a linker. In other embodiments the silicon atom is linked to the ammonium groups via an alkylene group.

A non-limiting example include: Si-alkylene-[N⁺(R₁)(R₂)-alkylene]ₙ—N⁺(R₁)(R₂)(R₃), wherein n is between 1-200 and R₁, R₂, R₃ are described hereinabove. In other embodiments n is between 2-200. In other embodiments n is 1, 2, 3, 4, 5 or 6. In other embodiments n is between 1-10, 2-10, 2-50, 2-5.

In some embodiments, provided herein an anti-microbial particle, comprising a polyhedral oligomeric silsesquioxane (POSS) core wherein each silicon atom of the POSS is functionalized by N-alkylated 3-(2-Aminoethylamino)propyl wherein the propyl end group is attached to the silicon atom; or by at least one functional polymerizable group selected from an acrylate, an epoxy, an isocyanate and a vinyl group; wherein the number of quaternary ammonium groups per silicon atom is between 2 to 200 and wherein the molar ratio of the silicon atom functionalized by N-alkylated 3-(2-Aminoethylamino)propyl and the silicon atom functionalized by at least one functional polymerizable group is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility, as well as subranges thereof, represents a separate embodiment of this invention.

In some embodiments, the silicon atom of the POSS (core of the anti-microbial particle) is functionalized by at least one functional polymerizable group, wherein the functional polymerizable group comprises an acrylate, an epoxy, isocyanate or a vinyl group. In one embodiment, “A silicon atom of the POSS (core of the anti-microbial particle) functionalized by at least one functional polymerizable group” refers to a silicon atom of the POSS (core) which is linked to an acrylate, an epoxy, isocyanate or a vinyl group by a linker. In other embodiments the Si is linked to the acrylate, epoxy or vinyl via an alkylene group. A non-limiting example of such silicon atom functionalization may include: Si-alkylene-O—C(O)—CH═CH₂ (Si-acrylate);

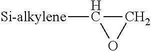

(Si-epoxy); Si-alkylene-CH═CH₂ (Si-vinyl) or Si-alkylene-N═C═O (Si-isocyanate). In some embodiments, the “Si” of said “(acrylate)”; “(epoxy)”; “(vinyl)”; or “(isocyanate)” structures is an integral part of the inorganic core and said Si atom is bonded/connected covalently to three oxygen atoms of said core (besides the covalent bonding/connection to “alkylene”). Each possibility representing a separate embodiment of this invention.

In some embodiments, when the core is inorganic, the polymerizable unit is Si-alkylene-O—C(O)—CH═CH₂ (Si-acrylate);

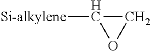

(Si-epoxy); Si-alkylene-CH═CH₂ (Si-vinyl) or Si-alkylene-N═C═O (Si-isocyanate), wherein “Si” is a part of siloxane moiety connected to the core directly.

In some embodiments, provided herein is an anti-microbial particle, comprising a polyhedral oligomeric silsesquioxane (POSS) core wherein each silicon atom of the POSS is functionalized by at least one quaternary ammonium group or by propyl acrylate, wherein the propyl end group is linked to the silicon atom; wherein the number of quaternary ammonium groups per silicon atom is between 2 to 200 and wherein the molar ratio of silicon atom functionalized by at least one quaternary ammonium and the silicon atom functionalized by propyl acrylate is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility, as well as subranges thereof, represents a separate embodiment of this invention.

In some embodiments, provided herein an anti-microbial particle, comprising a polyhedral oligomeric silsesquioxane (POSS) core wherein each silicon atom of the POSS is functionalized by N-alkylated 3-(2-Aminoethylamino)propyl wherein the propyl end group is attached to the silicon atom; or by propyl acrylate wherein the propyl end group is attached to the silicon atom; wherein the number of quaternary ammonium groups per silicon atom is between 2 to 200 and wherein the molar ratio of the silicon atom functionalized by N-alkylated 3-(2-Aminoethylamino)propyl and the silicon atom functionalized by propyl acrylate is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility, as well as subranges thereof, represents a separate embodiment of this invention.

In some embodiments, the anti-microbial particle provided herein comprises an inorganic or organic core, wherein the molar ratio of the anti-microbial active unit and the polymerizable unit is between 10:1 to 1:10, respectively. In other embodiments, the ratio is 4:1 to 1:1, respectively. In various embodiments, the ratio is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1 or 10:1 respectively, or any ranges or subranges thereof. In other embodiments, the anti-microbial unit comprises an anti-microbial group comprising a tertiary amine or a tertiary ammonium; or a quaternary ammonium and/or a pyridinium, as represented by the following formulas:
—⁺N(R₁)(R₂)(R₃), —⁺N(R₁)(R₂)-, —⁺NH(R₁)(R₂), —⁺NH(R₁)-, —N(R₁)(R₂), —N(R₁)-, —⁺N(R₁′)(R₂′)(R₃′), +N(R₁′)(R₂′)-, +NH(R₁′)(R₂′), +NH(R₁′)-, —N(R₁′)(R₂′), —N(R₁′)-,

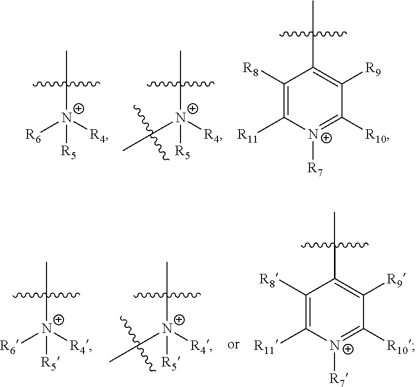

wherein:
R₁-R₁₁ and R₁′-R₁₁′ are as described hereinabove.

In one other embodiment, the activity of the quaternary ammonium, the tertiary amine, the tertiary ammonium or the pyridinium remains strong at any pH.

In some embodiments, the anti-microbial particle provided herein comprises a POSS core, wherein the molar ratio of the silicon atom (within the POSS) functionalized by at least one anti-microbial group and the silicon atom (within the POSS) functionalized by at least one functional polymerizable group is between 10:1 to 1:10, respectively. In other embodiments, the ratio is 4:1 to 1:1, respectively. In various embodiments, the ratio is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1 or 10:1 respectively, or any ranges or subranges thereof. In other embodiments, the anti-microbial group comprises, a tertiary amine or a tertiary ammonium; or a quaternary ammonium and/or a pyridinium, as represented by the following formulas:
—⁺N(R₁)(R₂)(R₃), —⁺N(R₁)(R₂)-, —⁺NH(R₁)(R₂), —⁺NH(R₁)—, —N(R₁)(R₂), —N(R₁)-, —⁺N(R₁′)(R₂′)(R₃′), —N(R₁′)(R₂′)-, —⁺NH(R₁′)(R₂′), —⁺NH(R₁′)-, —N(R₁′)(R₂′), —N(R₁′)-,

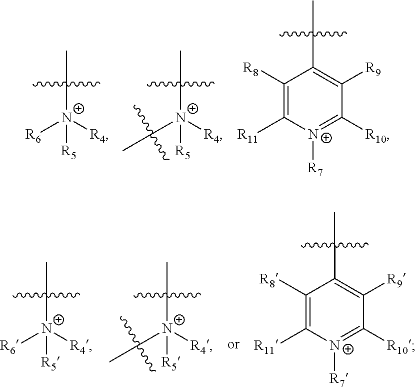

wherein:
R₁-R₁₁ and R₁′-R₁₁′ are as described hereinabove.

In one other embodiment, the activity of the quaternary ammonium, the tertiary amine, the tertiary ammonium or the pyridinium remains strong at any pH.

In some embodiments, provided herein an anti-microbial particle comprising a polyhedral oligomeric silsesquioxane (POSS) core wherein each silicon atom of the POSS is functionalized by at least one anti-microbial group or by at least one functional polymerizable group wherein the functional polymerizable group comprises an acrylate, an epoxy, an isocyanate or a vinyl group; and wherein the molar ratio of the silicon atom functionalized by at least one quaternary ammonium and the silicon atom functionalized at least one functional polymerizable group is between 10:1 to 1:10, respectively. In another embodiment, the ratio is between 9:1 to 1:9. In another embodiment, the ratio is between 8:1 to 1:8. In another embodiment, the ratio is between 7:1 to 1:7. In another embodiment, the ratio is between 6:1 to 1:6. In another embodiment, the ratio is between 5:1 to 1:5. In another embodiment, the ratio is between 4:1 to 1:4. In another embodiment, the ratio is between 3:1 to 1:3. In another embodiment, the ratio is between 2:1 to 1:2. Each possibility, as well as subranges thereof, represents a separate embodiment of this invention.

In some embodiments, the silicon atom of the POSS (core) is linked to one or more quaternary or pyridinium ammonium groups, tertiary ammonium, or a tertiary amine by a linker. In other embodiments the Si is linked to the ammonium/amine groups via an alkylene group. A non-limiting example of a quaternary ammonium linked to the Si atom: Si-alkylene-[N⁺(R₁)(R₂)-alkylene]ₙ—N⁺(R₁)(R₂)(R₃), wherein n is between 1-200 and R₁, R₂, R₃ are described hereinabove. In another embodiment, the quaternary ammonium linked to the silicon atom is N-alkylated 3-(2-Aminoethylamino)propyl, wherein the propyl end is linked to the silicon atom. In other embodiments, the N-alkylated group comprises C₁-C₁₈ alkyl units. In other embodiments, the N-alkylated group comprises C₁-C₁₈ alkyl units and at least one alkyl unit is a C₄-C₈ alkyl.

In some embodiments, the number of quaternary ammonium groups (=anti-microbial) per one sq nm (nm²) of the POSS core surface is of between 0.01-20 anti-microbial units per one sq nm (nm²) of the core surface of the particle. In another embodiment, the number of quaternary ammonium groups (=anti-microbial) per one sq nm (nm²) of the POSS core surface is of between 0.01-15, 0.01-12, 0.01-10, 0.01-8, 0.01-5, 0.01-3, 0.001-2, 0.001-3, 0.001-5, 0.5-3 or 0.5-5 anti-microbial units per one sq nm (nm²) of the core surface of the particle, each range represents a separate embodiment of this invention. The surface density of active groups onto particle surface have proportional impact on its anti-microbial activity.

In one embodiment, the core of the anti-microbial particles of this invention is a polyhedral oligomeric silsesquioxane (POSS), wherein at least one silicon atom thereof is capped. In one other embodiment, a “capped” silicon within a POSS means that said Si atom is not connected/bonded covalently to an anti-microbial active unit or polymerizable unit, rather, it is connected/bonded covalently e.g., to hydrogen or an organic moiety, e.g., alkyl, aryl; etc. In another embodiment, the ratio capped Si atoms to non-capped Si atoms is 4:1 to 1:4. In another embodiment, the ratio is 3:1 to 1:3. In another embodiment, the ratio is 2:1 to 1:2. Each possibility represents a separate embodiment of this invention.

For example, up to 70% of the Si atoms may be capped, e.g., using methylated, butyl, benzyl and/or propyl groups, which may be substituted or non-substituted. In various embodiments any of 0%, 10%, 20%, 30%, 40%, 50%, 60%, 70%, or intermediate proportions of the Si atoms may be capped.

### Organic Cores

In some embodiments, the core of the anti-microbial particles is an organic core. In one embodiment, the organic core comprises at least one aliphatic polymer. An “aliphatic polymer” as used within the scope of this invention refers to a polymer made of aliphatic monomers that may be substituted with various side groups, including (but not restricted to) aromatic side groups. Aliphatic polymers that may be included in particles according to this invention comprise nitrogen atoms (as well as other heteroatoms) as part of the polymeric backbone. In one embodiment, the core of the particles is an organic polymeric core including an amine which can be substituted with R₁, R₂ and/or R₃ as defined for structure 1; or including an imine which is chemically modified to amine and then substituted with R₁, R₂ and/or R₃ as defined for structure 1. In one embodiment, the core of the particles is an organic polymeric core including amines, as anti-microbial groups, which can be substituted with R₄, R₅, R₆, R_{4′}, R_{5′} and/or R_{6′} as defined for structure I; or including an imine which is chemically modified to amine and then substituted with R₄, R₅, R₆, R_{4′}, R_{5′}, and/or R_{6′} as defined for structure I. In other embodiments, the anti-microbial group comprises a tertiary amine or a tertiary ammonium; or a quaternary ammonium and/or a pyridinium, as represented by the following formulas:
—⁺N(R₁)(R₂)(R₃), —⁺N(R₁)(R₂)-, —⁺NH(R₁)(R₂), —⁺NH(R₁)—, —N(R₁)(R₂), —N(R₁)-, —⁺N(R₁′)(R₂′)(R₃′), —⁺N(R₁′)(R₂′)—, —⁺NH(R₁′)(R₂′), —⁺NH(R₁′)—, —N(R₁′)(R₂′), —N(R₁′)—,

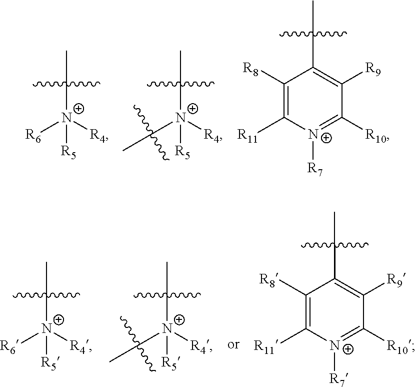

wherein:
R₁-R₁₁ and R₁′-R₁₁′ are as described hereinabove.

In one other embodiment, the activity of the quaternary ammonium, the tertiary amine, the tertiary ammonium or the pyridinium may remain strong at any pH.

In one embodiment, the functional polymerizable group comprises an acrylate, an epoxy, a vinyl or an isocyanate group.

In some embodiments, when the core is organic, the polymerizable unit comprises {N}-alkylene-O—C(O)—CH═CH₂ (N-acrylate);

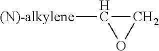

(N-epoxy); {N}-alkylene-CH═CH₂ (N-vinyl) or {N}-alkylene-N═C═O (N-isocyanate); wherein “Si” is a part of siloxane moiety connected to the core directly; and “{N}” is a nitrogenous (comprising nitrogen atom) linker, e.g., amide, amine, guanidine, imidazole etc.

Non-limiting examples of aliphatic polymers are polystyrene (PS), polyvinylchloride (PVC), polyethylene imine (PEI), polyvinyl amine (PVA), poly(allyl amine) (PAA), poly(aminoethyl acrylate), polypeptides with pending alkyl-amino groups, chitosan and co-polymers (combinations) thereof. Each possibility represents a separate embodiment of this invention. In one embodiment, the polymer is polyethylene imine (PEI).

In another embodiment, the organic core comprises at least one aromatic polymer selected from the following group: polystyrene, aminomethylated styrene polymers, aromatic polyesters, polyethylene terephthalate, and polyvinyl pyridine. For example, at least one aromatic polymer may comprise polyethylene terephthalate.

In another embodiment, the polymeric core may be linked to anti-microbial active unit directly (e.g., in structures (1)-(3): L₃ is a bond) or via a linker. In another embodiment, the polymeric core may be linked to anti-microbial active unit directly (e.g., in structures (I), (IE) and (II)-(VII): L₆ is a bond) or via a linker. Each possibility represents a separate embodiment of this invention.

In another embodiment, the polymeric core may be linked to the polymerizable unit directly (e.g., in structures (7): L₈ is a bond) or via a linker. Each possibility represents a separate embodiment of this invention.

In one embodiment, the organic polymeric core includes a combination of two or more different organic polymers. In another embodiment, the organic polymeric core includes a copolymer.

Resin/Composite Comprising Particles of this Invention

In some embodiments, the anti-microbial particles of this invention may be mixed with a polymeric matrix to form a resin/composite. In some embodiments, the functional polymerizable groups within the particles interact with the matrix and prevent leaching of the particles from the resin/composite. In one embodiment, the leaching is further prevented or reduced upon polymerization (e.g., in vivo) of the functional polymerizable groups.

In some embodiments, the ratio between the anti-microbial active units and the polymerizable units may be in the range between 10:1 to 1:10 or more specifically in the range between 4:1 to 1:1. Surprisingly, such ratios reduced the leakage of the particles from the composite by 1%, 10%, 20%, 30%, 40%, 50%, 60%, 70%, 80%, 90% or intermediate values, compared to anti-microbial particles which do not include polymerizable units. In some embodiment, the ratio between the silicon atom of the POSS (core) linked to an anti-microbial group (e.g., quaternary ammonium, tertiary amine, tertiary ammonium) and the silicon atom of the POSS (core) linked to the functional polymerizable group (e.g., acrylate, epoxy, vinyl, isocyanate) may be in the range between 10:1 to 1:10 or more specifically in the range between 4:1 to 1:1. Surprisingly, such ratios reduced the leakage of the particles from the composite by 1%, 10%, 20%, 30%, 40%, 50%, 60%, 70%, 80%, 90% or intermediate values, compared to anti-microbial particles which do not include the functional polymerizable groups.

The particles of this invention demonstrate an enhanced anti-microbial activity. Without being bound by any theory or mechanism, it can be postulated that such activity originates from the presence of closely packed anti-microbial groups on a given core's surface, as well as high density of particles packed on the surface of a host material. This density increases as each anti-microbial active unit in the particles of this invention comprise increasing number of anti-microbial groups and it yields a high local concentration of active functional polymerizable groups, which results in high effective concentration of the anti-microbial groups and enables the use of a relatively small number of particles to achieve effective bacterial/microbial annihilation. The close packing of the anti-microbial groups is due to, inter alia, numerous anti-microbial active units protruding from each particle surface. Accordingly, the anti-microbial groups cover large fraction of the particle's available surface area (width dimension covering the surface). The surface density of the anti-microbial group results in high effective concentration promoting anti-microbial inhibitory effect. According to the principles of this invention, high surface density dictates high anti-microbial efficiency.

The anti-microbial group of this invention may be in the form of a tertiary amine, or in the form of a protonated said tertiary amine, or in the form of a quaternary ammonium salt, or in the form of a pyridinium salt, as described hereinabove. Since an ammonium/pyridinium group is positively charged, its charge is balanced with an anion. In certain embodiments, in a particle according to this invention this anion is a halide, e.g., fluoride, chloride, bromide or iodide. For example, the anion may be fluoride. Other possible anions include, but are not limited to, bicarbonate, nitrate, phosphate, acetate, fumarate, succinate and sulfate. Each possibility represents a separate embodiment of this invention.

The core of the particle as described above may generally be in a form selected from a sphere, amorphous polygonal, shallow flake-like and a rod. In some embodiments, the core is spherical and has a diameter between about 5 to about 100,000 nm. In some embodiments, the core is spherical and has a diameter between about 1000-100,000 nm. In some embodiments, the core is spherical and has a diameter between about 100-1000 nm. In some other embodiments, the particles are in a size of about 20 to about 200 nm. In some other embodiments, the cores are in a size of about 20 to about 200 nm. In some other embodiments, the diameter of the cores may be in a size of about 20 to about 200 nm. In another embodiment, the diameter of the cores may be in a size of about 20 to about 200 nm. In one embodiment, the core is POSS and it is in a form selected from a sphere, amorphous polygonal, shallow flake-like and a rod. In some representative embodiments, the POSS core is spherical and has a diameter between about 5 to about 100,000 nm. In some representative embodiments, the POSS core is spherical and has a diameter between about 1000-100,000 nm. In some representative embodiments, the POSS core is spherical and has a diameter between about 100-1000 nm. In some other embodiments, the particles are in a size of about 20 to about 200 nm. In some other embodiments, the cores are in a size of about 20 to about 200 nm. In some other embodiments, the inorganic core is POSS and the particles are in a size of about 20 to about 200 nm. In another embodiment, the inorganic core is POSS and the core diameter is in a size of about 20 to about 200 nm. Each possibility represents a separate embodiment of this invention.

Specific Compositions Comprising the Particles of this Invention

Some embodiments include a composition comprising disclosed anti-microbial particles of one or more type and a polymeric material/resin as a matrix. For example, the particles may be homogeneously dispersed in the polymeric material (matrix). In some embodiments, the polymeric material may comprise organic polymers, inorganic polymers or any combination thereof. Organic polymers may comprise hydrogels, polyolefins, epoxy resin, acrylate resin, or any combination thereof. Inorganic polymers may comprise silicone polymers ceramics, metals or any combination thereof.

In various embodiments, the weight ratio of the particles to the polymeric material may be any of: between 0.25-5 wt %, 0.25-1 wt %, 1-2 wt %, 2-3 wt %, 3-4 wt %, 4-5 wt %, or within other subranges thereof.

In some embodiments, the composition provided herein is capable of filling of tooth decay cavities, is a dental restorative endodontic filling material for filling root canal space in root canal treatment, or is selected from the group consisting of a dental restorative material intended for provisional and final tooth restorations or tooth replacement, a dental inlay, a dental onlay, a crown, a partial denture, a complete denture, a dental implant, a dental implant abutment, and a cement intended for permanently cementing crowns bridges, onlays, partial dentures and orthodontic appliances onto tooth enamel and dentin.

In some embodiments, the composition provided herein, further comprises a filler. In other embodiments, the composition comprises 60-95% w/w filler and 10-30% w/w resin/polymeric material/matrix. In other embodiment, the composition comprises 78.5% w/w filler. In other embodiment, the composition comprises 20% w/w resin/polymeric material/matrix. In other embodiments, the composition comprises 78.5% w/w filler and 20% w/w resin/polymeric material/matrix.

In some embodiments, the resin is prepared by mixing bisphenol A glycidyl ether dimethacrylate (BisGMA), triethyleneglycol dimethacrylate (TEGDMA) and urethane dimethacrylate (UDMA), providing a clear blend; followed by mixing in camphoquinone (CQ), benzoin methyl ether (BME), ethyl 4-(dimethylamino) benzoate (EDB) and butylated hydroxytoluene (BHT), providing the resin.

In some embodiments, the composition provided herein comprises:
CQ 0.05-0.5% w/w;
BME 0.05-0.5% w/w;
EDB 0.05-0.5% w/w;
BHT 0.005-0.05% w/w;
BisGMA 3-10% ow/w;
TEGDMA 3-10% w/w;
UDMA 3-10% w/w;
Inorganic glass filler 60-95% w/w; and
Antimicrobial particles 0.25-5% w/w.

In some other embodiments, the composition provided herein comprises:
CQ 0.1% w/w;
BME 0.1% w/w;
EDB 0.1% w/w;
BHT 0.01% w/ww/w;
BisGMA 6.56% w/w;
TEGDMA 6.56% w/w;
UDMA 6.56% w/w;
Inorganic glass filler 78.5% w/w; and
Antimicrobial particles 1.5% w/w.

In some embodiments, the composition of this invention comprises the anti-microbial particles of this invention and a polymeric material comprising organic polymers, inorganic polymers or any combination thereof. In some embodiment, the particles as described herein are dispersed in the polymeric material. In another embodiment, the particles are homogeneously dispersed within the polymeric material. In another embodiment, the particles are found in the surface of the polymeric materials. In another embodiment, the particles interact covalently with the polymeric material. In another embodiment, the anti-microbial particles are mechanically embedded within the polymeric material. In another embodiment, these particles are three dimensionally “locked” between the polymer chains, preventing them from migrating out from the complex network. The strong hydrophobic nature of these particles also plays a role in preventing the particles from moving into the hydrophilic surrounds such as in the case of physiological, dental, orthopedic or other medical applications. In one embodiment, the particles comprise functional polymerizable groups, capable of reacting with moieties of the polymeric material. In another embodiment, the particles interact chemically with the polymeric material. In another embodiment, the particles comprise functional polymerizable groups which react with monomers to provide the polymeric material of the composition. In other embodiment, the particles comprise functional polymerizable groups which react with monomers to provide a polymer, in addition to (and/or with) the polymeric material of the composition. In one other embodiment, the reaction of the functional polymerizable group with the polymeric material and/or the monomers can be catalyzed and/or initiated by catalyst(s) and/or (photo)-initiator. In other embodiment, non-limiting examples of photo-initiators include: camphorquinone (CQ), benzoin methyl ether (BME) and ethyl 4-(dimethylamino) benzoate (EDB). In another embodiment, the particles are a mixture of different particles.

In some embodiments, the composition of this invention comprises the anti-microbial particles of this invention and a polymeric material comprising organic polymers, inorganic polymers or any combination thereof. In another embodiment, the polymeric material comprises thermoplastic polymers, thermoset polymers or any combination thereof. In another embodiment, the organic polymer comprises hydrogels, polyolefins such as polyvinylchloride (PVC), polyethylene, polystyrene and polypropylene, epoxy resins, acrylate resins such as poly methyl methacrylate, polyurethane or any combination thereof. In another embodiment, the inorganic polymer comprises silicone polymers such as polydimethylsiloxane (PDMS), ceramics, metals or any combination thereof. In another embodiment, the hydrogel is poloxamer or alginate. In another embodiment, the commercial poloxamer is used or it is formed by a reaction between a polymer and other reagent. In another embodiment, the polymer is poly(ethylene glycol) (PEG) with reactive end groups (such as epoxides in PEG-diglycidyl ether) and the reagent has multiple reactive sites (e.g., diethylenetriamine). Each possibility represents a separate embodiment of this invention.

In some embodiments, the weight ratio of the particles to the polymeric material is between 0.25-5%. In another embodiment, the weight ratio is between 0.5-2%. In another embodiment, the weight ratio is between 1-5%.

Another polymer material to be used in the context of this invention is resins used in dental, surgical, chirurgical and orthopedic composite materials. In such applications, anti-microbial particles could be first dispersed within the resin part or added simultaneously with filler or any other solid ingredients (if any). Most of these resins are acrylic or epoxy type monomers that undergo polymerization in-vivo. In some embodiment, the antimicrobial particles also undergo polymerization in-vivo (regardless of the identity or presence of the additional polymeric material of the matrix).

### Processes of Preparing the Anti-Microbial Particles

### Preparation of Anti-Microbial Particles, Comprising One Monomeric Unit Per One Anti-Microbial Active Unit

The particles of this invention may be prepared in accordance to a variety of processes, depending on the nature of the core, the anti-microbial active group, and the presence or absence of linkers. Some non-limiting examples of preparation methods are provided below.

In one embodiment, this invention provides processes for preparing anti-microbial particles, wherein the particles comprise at least one anti-microbial active unit and at least one polymerizable unit, wherein one monomeric unit is found per one anti-microbial active unit. In one further embodiment, in the particles prepared by said processes—the ratio between the number of anti-microbial units and polymerizable units is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1 or 10:1 respectively, or any ranges or subranges thereof. In the following, such processes will be presented in detail.

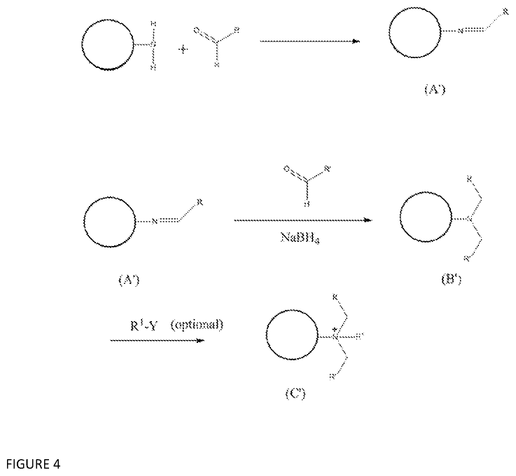

A representative method for preparing particles of this invention wherein the anti-microbial active group is a tertiary amine or a quaternary ammonium group comprising at least one terpenoid moiety is represented in FIG. 4, for standard particles. In accordance with FIG. 4, a core as defined herein is functionalized with a primary amine, and optionally said core already includes (at least one) polymerizable unit connected/bonded covalently (directly or indirectly via a linker as described herein) thereto. The primary amine reacts with an aldehyde to yield initially an imine (Schiff base) intermediate of formula (A′), which is then reacted with a second aldehyde under reductive amination conditions to yield a tertiary amine of formula (B′). RC(═O)H and R′C(═O)H each represent an aldehyde which is a terpenoid or which is derived from aterpenoid. RC(═O)H and R′C(═O)H may be the same or different from each other. Conversion of the tertiary amine to the quaternary ammonium group is optional, and involves reaction of the tertiary amine with a group R¹—Y wherein R¹ is a C₁-C₄ alkyl and Y is a leaving group such as halogen or sulfonate. In one embodiment, said core already includes (at least one) polymerizable unit connected/bonded covalently (directly or indirectly via a linker as described herein) thereto. In one other embodiment, the particles resulting from the above processes as depicted in FIG. 4 (B′ or C′) are further reacted to afford the antimicrobial particles of this invention, comprising at least one anti-microbial active unit and at least one polymerizable unit.

It is understood that the group

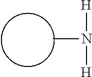

may represent any one or more of the following:
1. An organic core directly bound to NH₂ and, optionally directly bound to polymerizable unit or optionally indirectly bound to the same (polymerizable unit) through a linker as described herein.
2. An organic core bound to NH₂ through a linker as described herein and optionally directly bound to polymerizable unit or optionally indirectly bound to the same (polymerizable unit) through a linker as described herein.
3. An inorganic core directly bound to NH₂ and optionally directly bound to polymerizable unit or optionally indirectly bound to the same (polymerizable unit) through a linker as described herein.
4. An inorganic core bound to NH₂ through a linker as described herein and optionally directly bound to polymerizable unit or optionally indirectly bound to the same (polymerizable unit) through a linker as described herein.

The exemplified reaction (FIG. 4) may be a “one pot synthesis”, or it may include two sequential reactions with isolation of an intermediate formed in the first step. The first step is the formation of intermediate (A′), which is an imine (Schiff base), by reacting an amine functionalized core with a terpenoid moiety in the presence of a reducing agent, in this case cinnamyl in the presence of NaBH₄. The imine functionalized core can be isolated at this stage if desired. Alternatively, further reacting intermediate (A′) with a terpenoid moiety in the presence of a reducing agent yields a tertiary amine comprising two terpenoid moieties (B′). In order to obtain the quaternary ammonium, additional alkylation step is performed as described in FIG. 4.

Particles with enhanced thermal stability can be prepared in a similar fashion as described above and illustrated in FIG. 4 for standard particles, with a few notable differences: for standard particles R and R′ are terpenoid moieties, where for particles with enhanced thermal stability, R and R′ are each independently methyl, CF₃, perhaloalkyl, aryl, —C(═O)OR, —C(═O)OC(═O)R, —C(═S)OR, —C(═O)SR, —C(═O)—R, —C(═S)—R, 1-alkenyl or 1-alkynyl, where R is alkyl, aryl, cycloalkyl, heterocycle or any combination thereof; R¹ is a methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, where R is alkyl, aryl, cycloalkyl, heterocycle or any combination thereof. In this case of particles with enhanced thermal stability—the final reaction with R¹—Y is mandatory and not optional, in order to arrive at ammonium. In one embodiment, as noted above re. FIG. 4's processes, said core already includes (at least one) polymerizable unit connected/bonded covalently (directly or indirectly via a linker as described herein) thereto. In one other embodiment, the particles resulting from the above processes as depicted in FIG. 4 (C′) are further reacted to afford the antimicrobial particles of this invention, comprising at least one anti-microbial active unit and at least one polymerizable unit.

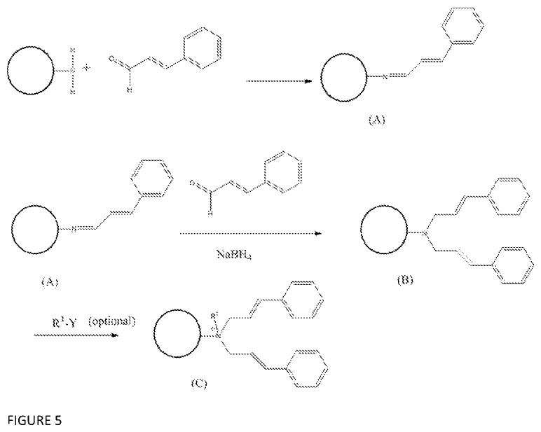

The process for the preparation of standard particles and presented in FIG. 5, uses cinnamaldehyde, but is applicable to other aldehydes. Thus, in some embodiments, this invention provides a particle comprising (i) an inorganic core or an organic polymeric core; (ii) an imine moiety chemically bound to the core, e.g., at a surface density of at least one imine group per 10 sq. nm, wherein the imine group comprises a terpenoid moiety; and optionally (iii) (at least one) polymerizable unit connected/bonded covalently (directly or indirectly via a linker as described herein) to the core. The imine moiety is generally represented by the structure of formula (A′) in FIG. 4. A more specific embodiment is the structure of formula (A) in FIG. 5. It is understood by a person of skill in the art that other imine intermediate compounds comprising other terpenoids groups as described herein, are also encompassed by this invention.

In one embodiment, the core in the processes of FIG. 5 already includes (at least one) polymerizable unit connected/bonded covalently (directly or indirectly via a linker as described herein) thereto. In one other embodiment, the particles resulting from the above processes as depicted in FIG. 5 (B or C) are further reacted to afford the antimicrobial particles of this invention, comprising at least one anti-microbial active unit and at least one polymerizable unit.

It is understood that that the group

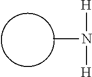

of FIG. 5 has any one of the meanings as described above for FIG. 4.

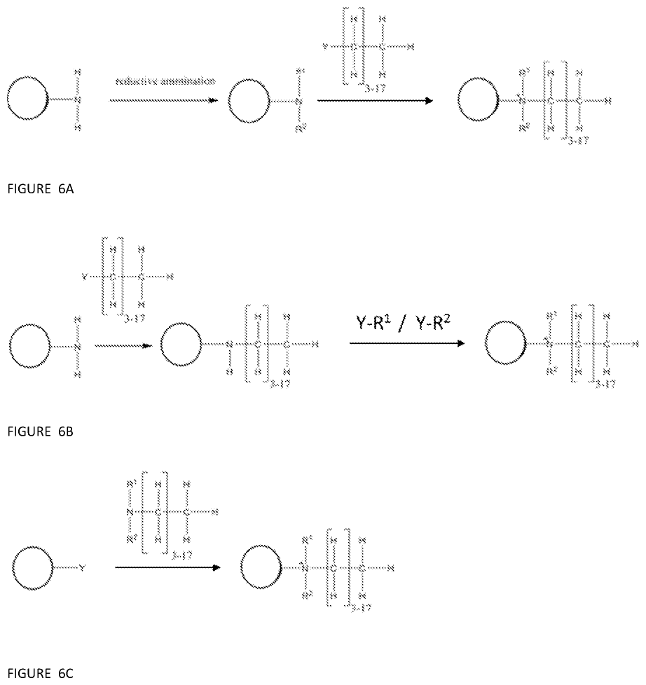

A representative method for preparing standard particles wherein the anti-microbial active group is a quaternary ammonium group containing one alkyl group having 4 to 18 carbon atoms is presented in FIGS. 6A-6C. The method includes three pathways to prepare quaternary ammonium salts (QAS) functionalized particle. FIG. 4A) by first utilizing reductive amination to achieve tertiary amine, followed by an alkylation reaction, FIG. 4B) by stepwise alkylation reactions; and FIG. 4C) by reacting a linker functionalized with a leaving group (e.g., Cl or other halogen) with tertiary amine. R¹ and R² represent C₁-C₄ alkyls such as methyl, ethyl, propyl or isopropyl. R¹ and R² may be different or the same group. Y represents any leaving group, for example Cl, Br or I, or a sulfonate (e.g., mesyl, tosyl). In one embodiment,

It is understood that that the group

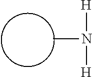

has any one of the meanings as described above for FIGS. 4A-4C and 5.

In one embodiment, the core in the processes of FIGS. 6A-6C already includes (at least one) polymerizable unit connected/bonded covalently (directly or indirectly via a linker as described herein) thereto. In one other embodiment, the particles resulting from the above processes as depicted in FIGS. 6A-6C are further reacted to afford the antimicrobial particles of this invention, comprising at least one anti-microbial active unit and at least one polymerizable unit. In some other embodiments, the further reactions are detailed hereinbelow in the sections titled “Solid support as method of preparation of anti-microbial particles comprising one monomeric unit per one anti-microbial active unit”; and “Solution method as method of preparation of anti-microbial particles comprising one monomeric unit per one anti-microbial active unit”.

It is understood that that the group

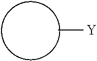

may represents any one or more of the following:
1. An organic core directly bound to Y and optionally directly bound to polymerizable unit or optionally indirectly bound to the same (polymerizable unit) through a linker as described herein.
2. An organic core bound to Y through a linker as described herein and optionally directly bound to polymerizable unit or optionally indirectly bound to the same (polymerizable unit) through a linker as described herein.
3. An inorganic core directly bound to Y and optionally directly bound to polymerizable unit or optionally indirectly bound to the same (polymerizable unit) through a linker as described herein.
4. An inorganic core bound to Y through a linker as described herein and optionally directly bound to polymerizable unit or optionally indirectly bound to the same (polymerizable unit) through a linker as described herein.

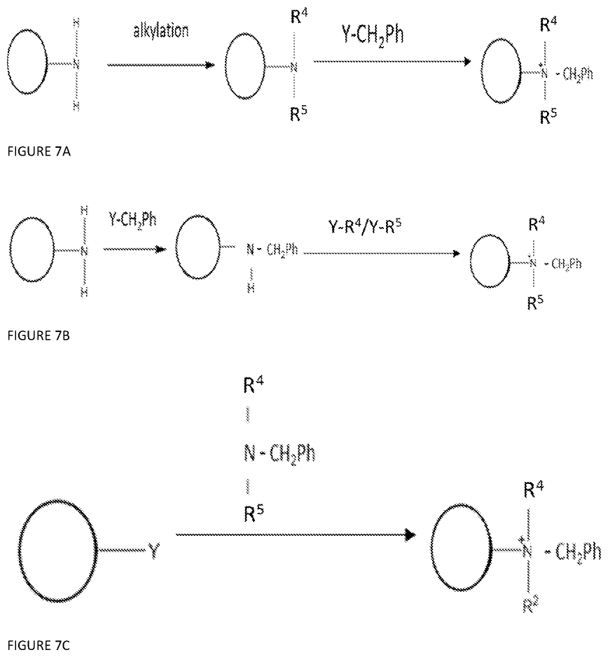

Similar method of preparing particles with enhanced thermal stability is represented in FIGS. 7A-7C. The method includes three pathways to prepare quaternary ammonium salts (QAS) functionalized particle. FIG. 7A) by reaction with R₁—Y/R₂—Y to achieve tertiary amine, followed by benzylation reaction; FIG. 7B) by a similar pathway as in FIGS. 7A), done in the reversed order; and FIG. 7C): by reacting a linker functionalized with a leaving group (e.g., Cl or other halogen) with tertiary amine. R₄ and R₅ are independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof. Y represents any leaving group, for example C₁, Br or I, or a sulfonate (e.g., mesyl, tosyl). In one embodiment, as noted above concerning the processes described in FIGS. 4-6, said core already includes (at least one) polymerizable unit connected/bonded covalently (directly or indirectly via a linker as described herein) thereto. In one other embodiment, the particles resulting from the above processes as depicted in FIGS. 7A-7C are further reacted to afford the antimicrobial particles of this invention, comprising at least one anti-microbial active unit and at least one polymerizable unit.

In some other embodiments, the further reactions are detailed hereinbelow in the sections titled “Solid support as method of preparation of anti-microbial particles comprising one monomeric unit per one anti-microbial active unit”; and “Solution method as method of preparation of anti-microbial particles comprising one monomeric unit per one anti-microbial active unit”.

Core functionalization can occur by a solid support method, or a solution method.

### Solid Support as Method of Preparation of Anti-Microbial Particles Comprising One Monomeric Unit Per One Anti-Microbial Active Unit

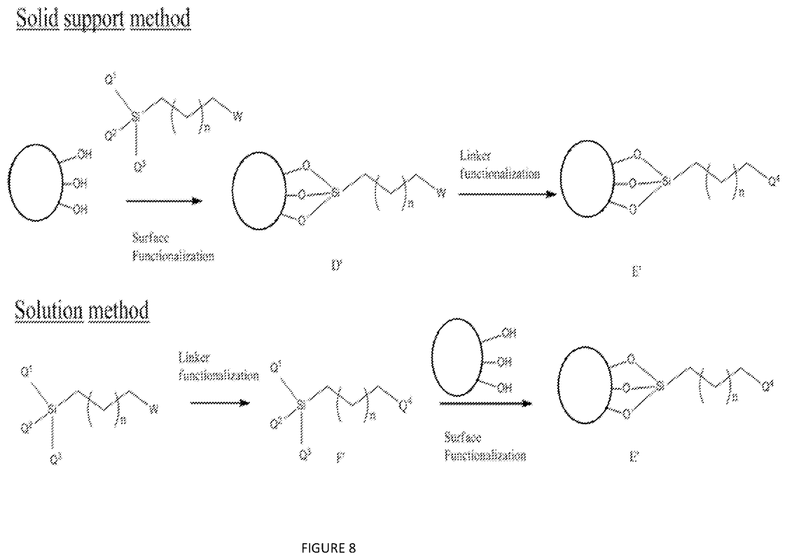

Preparation of functionalized standard particles is conducted in two general steps. First, the linker molecule is allowed to condense onto particles surface (surface functionalization) via hydrolysis of leaving groups to give an intermediate of formula (FIG. 8, D′). Second, functional sites of the linker molecule undergo further functionalization (linker functionalization) as mentioned in any ones of (FIGS. 4-7) to give a functionalized particle of formula E′ of FIG. 8. The circles in FIG. 8 represent an organic or inorganic core; Q¹, Q² and Q³ are independently selected from the group consisting of ethoxy, methoxy, methyl, ethyl, hydrogen, sulfonate and halide, wherein at least one of Q¹, Q² and Q³ is a leaving group selected from ethoxy, methoxy, sulfonate (e.g., mesyl, tosyl) and halide; W is selected from the group consisting of NH₂, halide, sulfonate and hydroxyl; and n is an integer between 1 and 16. For the sake of clarity the scheme presents a case where Q¹, Q² and Q³ represent leaving groups; Q⁴ represents an anti-microbial group. Similar process is used for the preparation of functionalized particles with enhanced thermal stability with the difference that W accommodates the same substituents with the exception that the NH₂ moiety is replaced with an arylene-NH₂ or benzylene-NH₂ moiety. In one embodiment, the polymerizable unit is added in a similar manner to this solid support processes of FIG. 8; in this case, W of

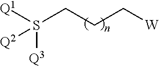

is the functional polymerizable group; Q¹-Q³ are as indicated hereinabove; and said polymerizable unit addition (surface functionalization step) is performed before or after the functionalization of the core (surface+linker functionalizaion) with the anti-microbial units. In another embodiment, the second step of linker functionalization is not performed when said polymerizable unit is added to the particles, e.g., only the step of “surface functionalization” is employed when the polymerizable unit is introduced to the cores/particles.

### Solution Method as Method of Preparation of Anti-Microbial Particles Comprising One Monomeric Unit Per One Anti-Microbial Active Unit

In this method, the linker molecule is first functionalized with antimicrobial active group to give an intermediate of formula (FIG. 8, F′). In the second stage intermediate (F′) is allowed to settle onto particle's solid surface (surface functionalization) to give a functionalized particle of formula (FIG. 8, E′).

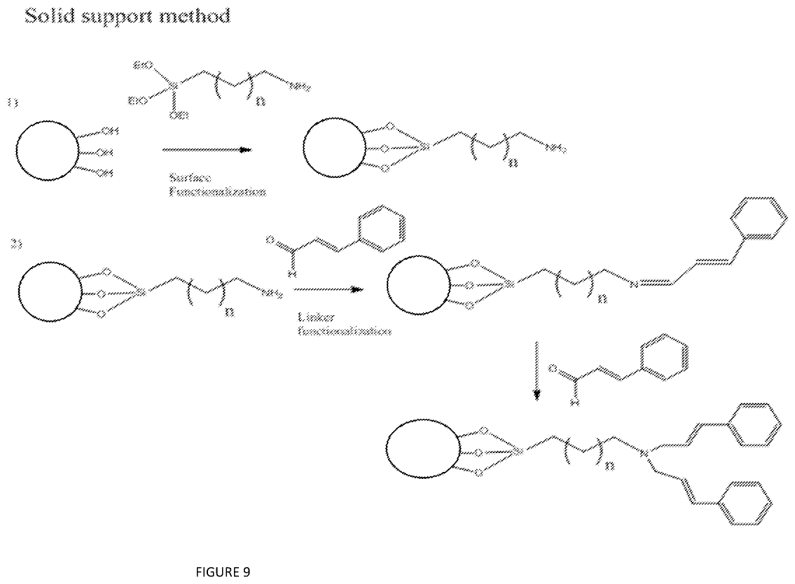

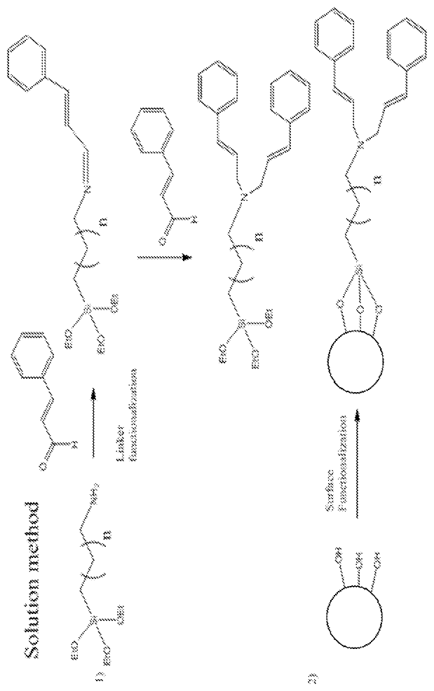

This process is exemplified in FIG. 9 for cinnamaldehyde standard particles, but is applicable to other aldehydes.

Similar process is used for the preparation of functionalized particles with enhanced thermal stability with the difference that W accommodates the same substituents with the exception that the NH₂ moiety is replaced with an arylene-NH₂ or benzylene-NH₂ moiety. In one embodiment, the polymerizable unit is added in a similar manner to this solution support processes of FIGS. 8-9; in this case, W of

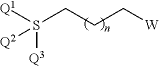

is the functional polymerizable group; Q¹-Q³ are as indicated hereinabove; and said polymerizable unit addition (surface functionalization step) is performed before or after the functionalization of the core (surface+linker functionalizaion) with the anti-microbial units. In another embodiment, the first step of linker functionalization is not performed when said polymerizable unit is added to the particles, e.g., only the step of “surface functionalization” is employed when the polymerizable unit is introduced to the cores/particles.
Preparation of Anti-Microbial Particles, Comprising More than One Monomeric Unit Per One Anti-Microbial Active Unit

In one embodiment, this invention provides processes for preparing particles of the composites of this invention, wherein the particles comprise more than one monomeric unit per one anti-microbial active unit In one further embodiment, in the particles prepared by said processes—the ratio between the number of anti-microbial units and polymerizable units is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1 or 10:1 respectively, or any ranges or subranges thereof. In the following, such processes will be presented in detail.

Solid Support as Method of Preparation of Anti-Microbial Particles Comprising More than One Monomeric Unit Per One Anti-Microbial Active Unit

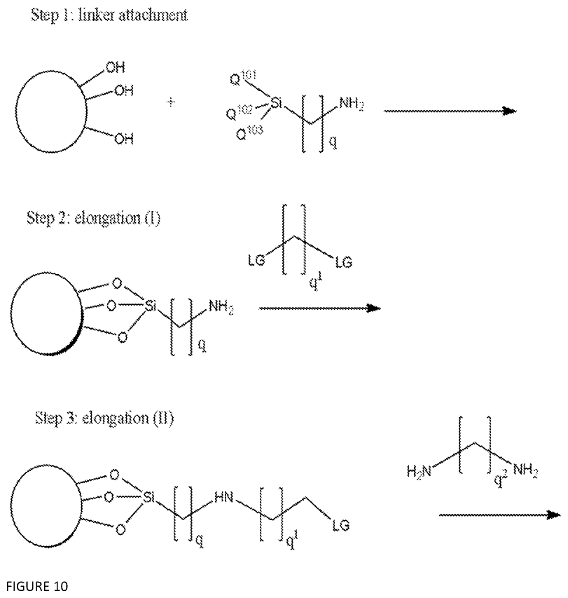

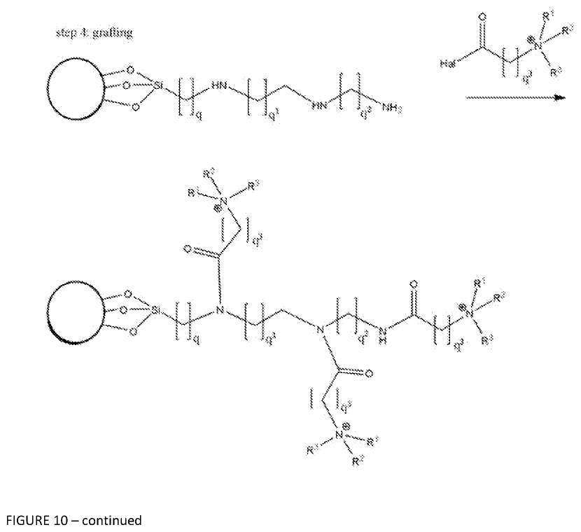

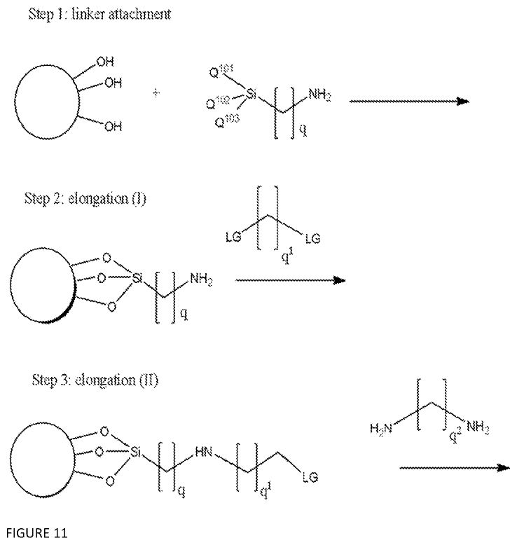

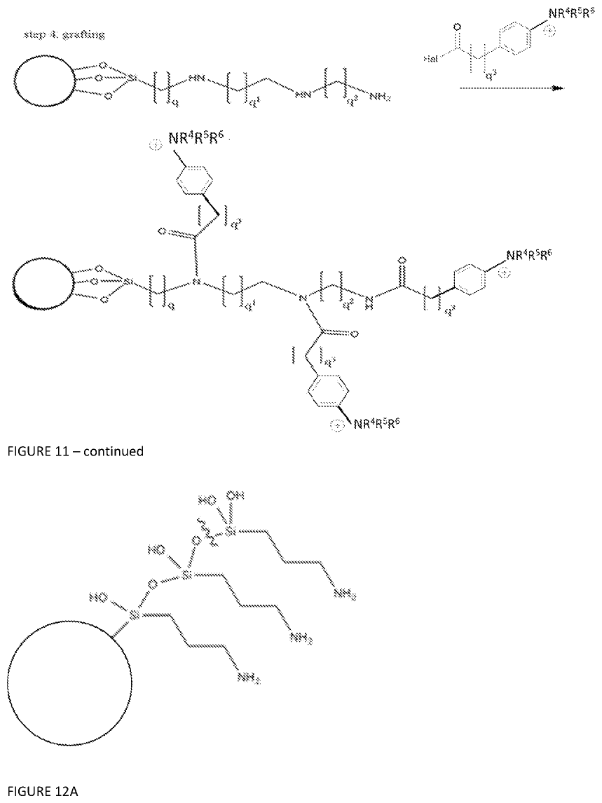

The solid support method comprises a few stages. First, for standard particle, the linker molecule (dilute solutions of a few percent) is allowed to condense onto particles surface (surface functionalization) via (acid catalyzed) hydrolysis of leaving groups, resulting in the attachment of the linker to the core (FIG. 10, step 1). Second, the attached linker is elongated. In another embodiment, this stage is achieved synthetically via one step or more. In another embodiment, elongation is achieved by consecutive addition of difunctionalized alkane and diaminoalkane, wherein amines (of attached linker and diaminoalkane) attack electrophilic centers of the difunctionalized alkane (FIG. 10, steps 2 and 3). In another embodiment, such consecutive addition is optionally repeated for 1-10 times. Finally, the anti-microbial active group (usually attached to an alkylene chain) is grafted to resulting attached and elongated linker. In another embodiment, grafting is accomplished when amines on the attached and elongated linker attack acyl halide moiety of the molecule of the anti-microbial active group which is grafted (FIG. 10, step 4). Similar process is presented for particles with enhanced thermal stability (FIG. 11), where the ammonium end of the anti-microbial active group is replaced with an anilinium end and R¹—R are replaced with R⁴-R⁶. In FIG. 10, R¹ and R² are each independently alkyl, terpenoid, cycloalkyl, aryl, heterocycle, a conjugated alkyl, alkenyl or any combination thereof, and R³ is nothing, hydrogen, alkyl, terpenoid moiety, cycloalkyl, aryl, heterocycle, alkenyl, alkynyl or any combination thereof. In FIG. 11, R₄ and R₅ are each independently methyl, CF₃, perhaloalkyl, aryl, benzyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, 1-alkenyl, 1-alkynyl, 2-alkenyl, 2-alkynyl or any combination thereof, and R₆ is methyl, CF₃, perhaloalkyl, 2,2-disubstituted C₃-C₂₀ alkyl, 2,2,2-trisubstituted ethyl, —CH₂C(═O)OR, —CH₂C(═O)OC(═O)R, —CH₂C(═S)OR, —CH₂C(═O)SR, —C(═O)OR, —C(═O)OC(═O)R, —C(═S)OR, —C(═O)SR, —C(═O)—R, —C(═S)—R, —CH₂C(═O)R, —CH₂C(═S)R, —CH₂CF₃, —CH₂NO₂, terpenoid moiety, cycloalkyl, aryl, phenyl, benzyl, heterocycle, a conjugated alkyl, 1-alkenyl, 1-alkynyl, 2-alkenyl, 2-alkynyl or any combination thereof.

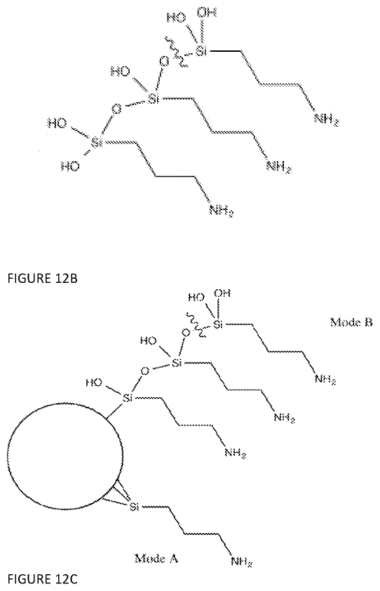

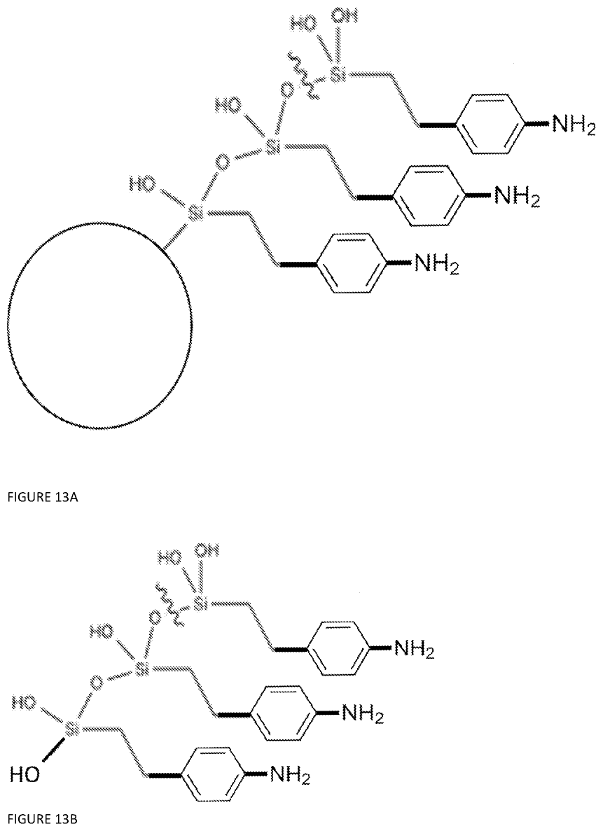

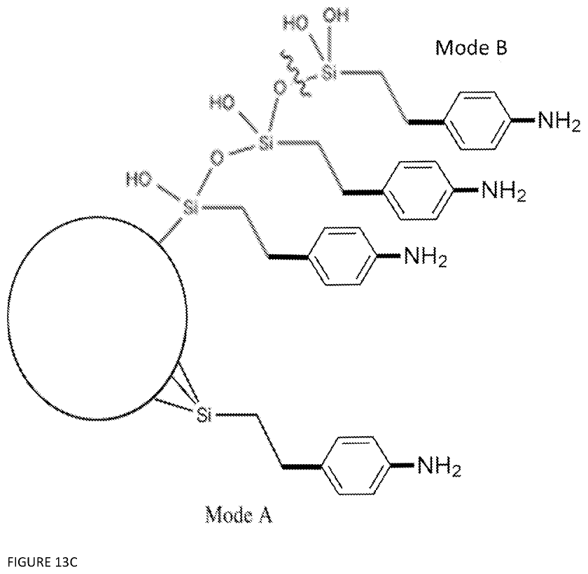

In another embodiment, the same trialkoxysilane linker molecule (of FIGS. 12-13) is used initially, however in a higher concentration (≥10% by wt) and it initially self-polymerizes (FIGS. 12A and 13A for standard and thermally stable enhanced particles, respectively) under basic catalysis. Functionalization of the solid supported linker progresses similarly as in the procedures described hereinabove for particles that comprise one monomeric unit per one anti-microbial active unit (FIGS. 4-9).

In some embodiments, the core in the processes of FIGS. 10-13 already includes (at least one) polymerizable unit connected/bonded covalently (directly or indirectly via a linker as described herein) thereto. In one other embodiment, the particles resulting from the above processes as depicted in FIGS. 10-13 are further reacted to afford the antimicrobial particles of this invention, comprising at least one anti-microbial active unit and at least one polymerizable unit. In some other embodiments, the further reactions are detailed hereinabove in the sections titled “Solid support as method of preparation of anti-microbial particles comprising one monomeric unit per one anti-microbial active unit”; and “Solution method as method of preparation of anti-microbial particles comprising one monomeric unit per one anti-microbial active unit”.

Solution Method as Method of Preparation of Anti-Microbial Particles Comprising More than One Monomeric Unit Per One Anti-Microbial Active Unit

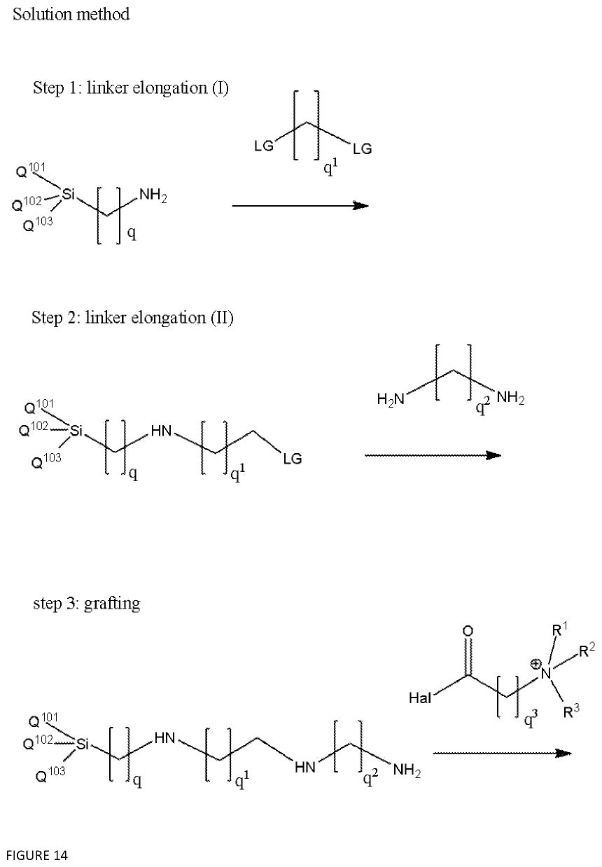

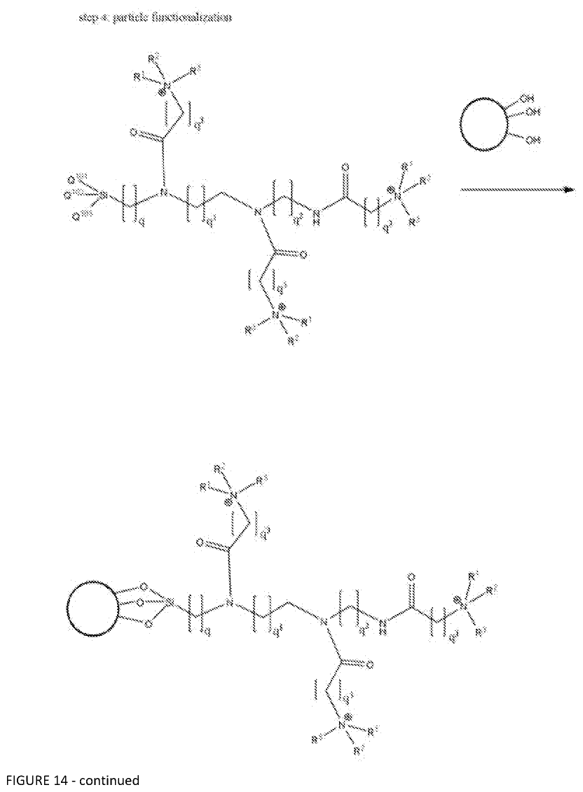

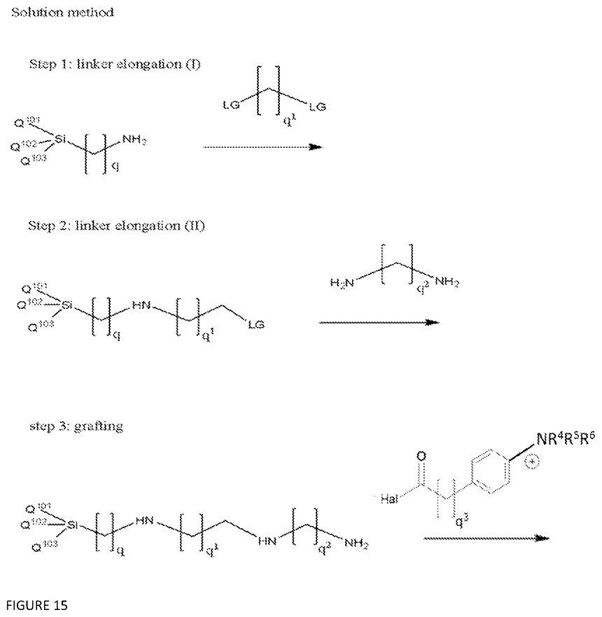

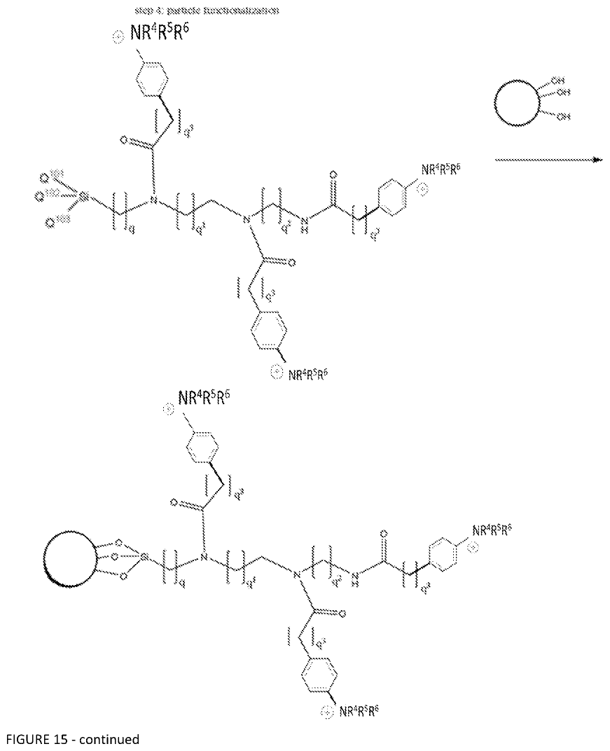

The solution method comprises a few stages. The first step involves elongation of the linker molecule. In another embodiment, this step is achieved synthetically via one step or more. In another embodiment, elongation is achieved by consecutive addition of difunctionalized alkane and diaminoalkane wherein amines (of linker and diaminoalkane) attack electrophilic centers of the difunctionalized alkane (FIGS. 14 and 15 for standard and thermally stable enhanced particles, respectively: steps 1 and 2). In another embodiment, such consecutive addition is optionally repeated for 1-10 times. In the second stage, the anti-microbial active group (usually attached to an alkylene chain) is grafted to resulting elongated linker. In another embodiment, grafting is accomplished when amines on the elongated linker attack acyl halide moiety of the molecule of the anti-microbial active group which is grafted (FIGS. 14 and 15, step 3). Finally, the elongated, anti-microbial active linker is attached to the core via functionalization thereof. In this step, the linker molecule (dilute solutions of a few percent) is allowed to condense onto particles surface (surface functionalization) via (acid catalyzed) hydrolysis of leaving groups, resulting in the attachment of the linker to the core (FIGS. 14 and 15, step 4).

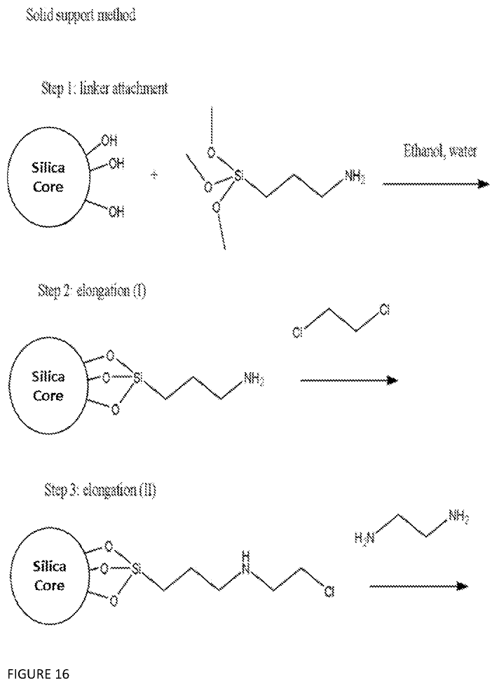

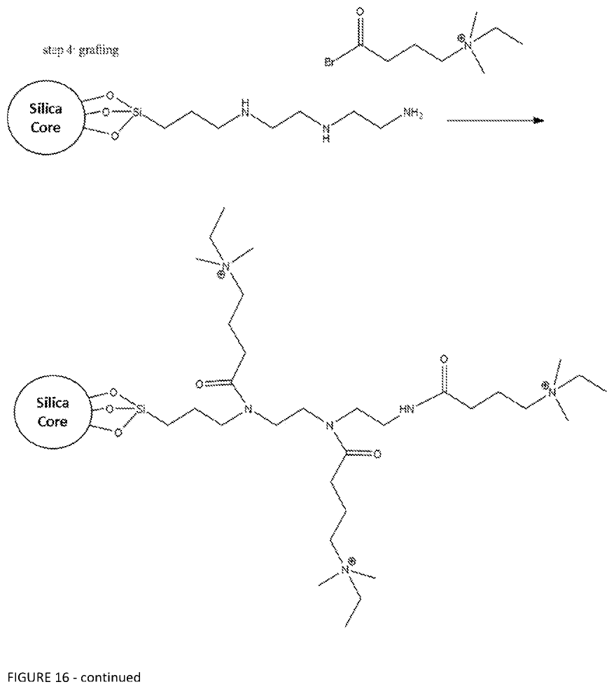

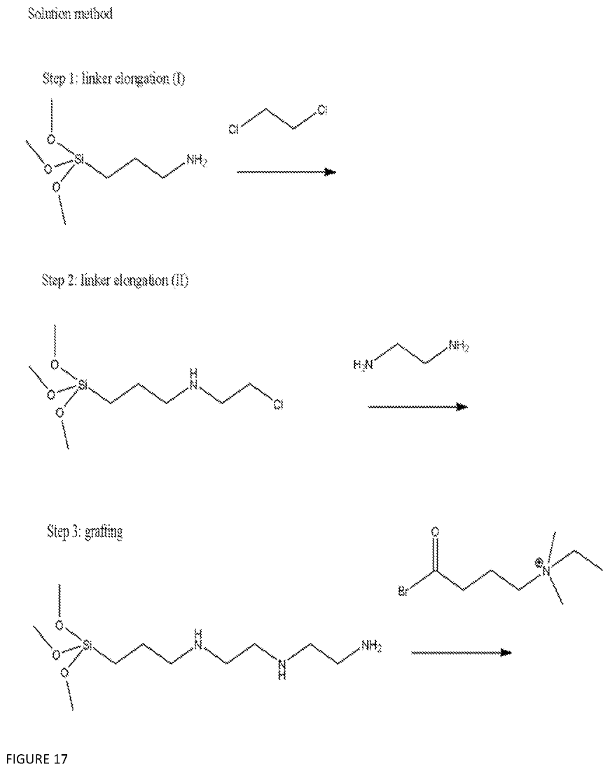

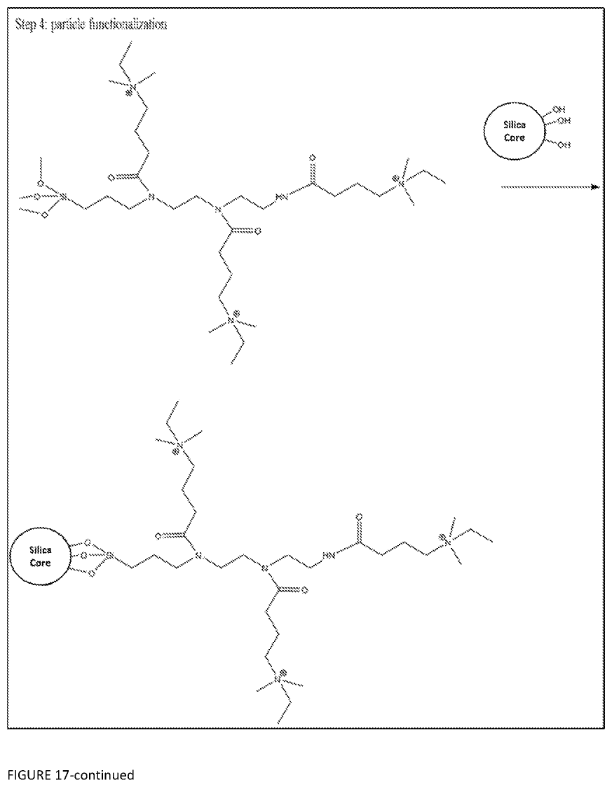

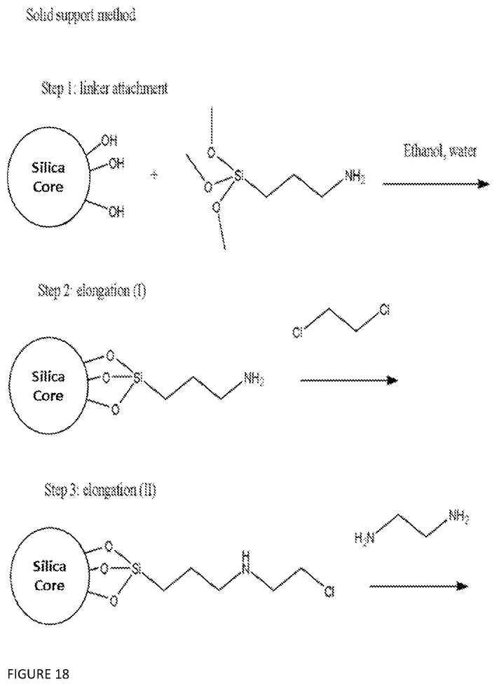

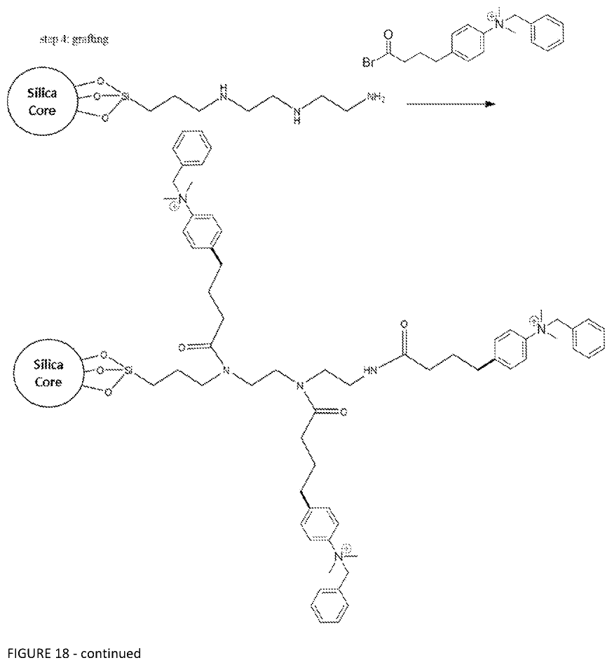

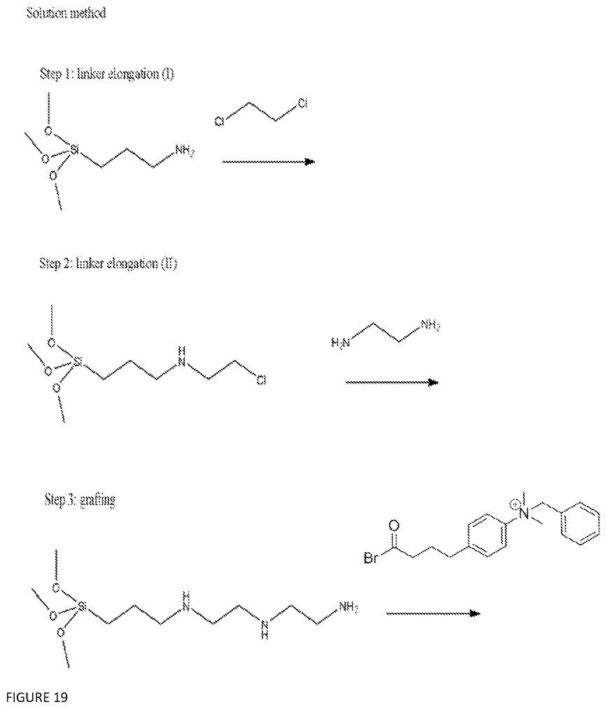

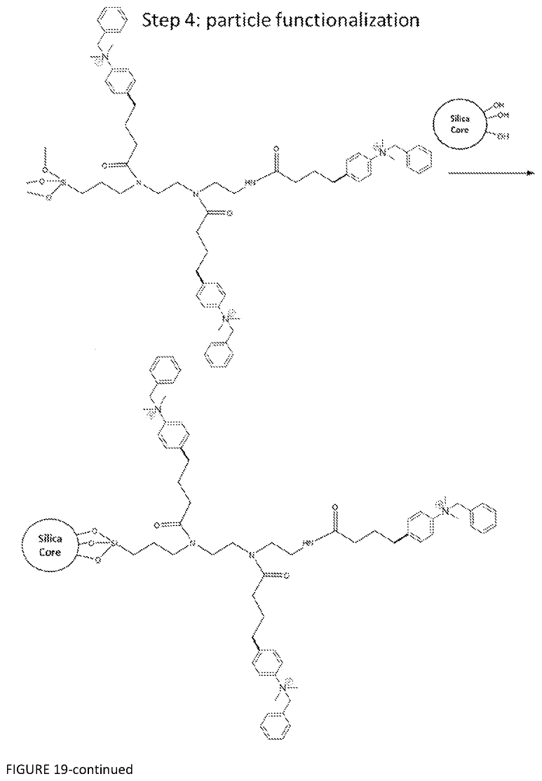

This process is exemplified in FIGS. 16-17 for silica standard particles functionalized with dimethylethylammonium, Similarly, the process is exemplified in FIGS. 18-19 for silica particles with enhanced thermal stability functionalized with dimethybenzylammonium, but is applicable to other hydroxyl-terminated cores and anti-microbial active groups. The processes of FIGS. 14-19 are applicable to other hydroxyl-terminated cores and anti-microbial active groups.

In another embodiment, the same trialkoxysilane linker molecule is used initially, however in a higher concentration (≥10% by weight) and it initially self-polymerizes (see, e.g., FIGS. 12B and 13B for standard and thermally stable enhanced particles, respectively) under basic catalysis. Functionalization of the linker progresses similarly as in the procedures described hereinabove for particles that comprise one monomeric unit per one anti-microbial active part (see, e.g., FIGS. 4-9).

In some embodiments, the core in the processes of FIGS. 14-19 already includes (at least one) polymerizable unit connected/bonded covalently (directly or indirectly via a linker as described herein) thereto. In one other embodiment, the particles resulting from the above processes as depicted in FIGS. 14-19 are further reacted to afford the antimicrobial particles of this invention, comprising at least one anti-microbial active unit and at least one polymerizable unit. In some other embodiments, the further reactions are detailed hereinabove in the sections titled “Solid support as method of preparation of anti-microbial particles comprising one monomeric unit per one anti-microbial active unit”; and “Solution method as method of preparation of anti-microbial particles comprising one monomeric unit per one anti-microbial active unit”.

In some embodiments, when it is noted herein that the core in the processes of FIGS. 4-19 already includes (at least one) polymerizable unit connected/bonded covalently (directly or indirectly via a linker as described herein) thereto—it is meant that said polymerizable unit was connected/bonded to the core via any one of the methods as described hereinabove, e.g., as in the sections titled “Solid support as method of preparation of anti-microbial particles comprising one monomeric unit per one anti-microbial active unit”; and “Solution method as method of preparation of anti-microbial particles comprising one monomeric unit per one anti-microbial active unit”.

In one embodiment, an anti-microbial particle is prepared according to any one of the processes as described hereinabove (having one or more monomeric unit(s) per one anti microbial unit), wherein the actual polymerizable functional group comprises a multiplicity of functional polymerizable groups; see Z₃ of Formula (7). In another embodiment, processes similar to the ones described hereinabove are provided to result in said antimicrobial particles having Z₃ comprising a multiplicity of functional polymerizable groups; Z₃ is attached as described above (where Z₃ “mono-functional” polymerizable groups).

### Preparation of Core Particles

In some embodiments, the particles of the composites of this invention which comprise one or more monomeric units per one anti-microbial active part, comprise cores which are prepared according to the following.

Porous silica materials can be prepared by reaction of SiCl₄ with alcohol or water, followed by drying using centrifugation and/or heating utilizing airflow or under vacuum conditions. Dense fumed silica particles (pyrogenic) were prepared by pyrolysis of SiCl₄.

An alternative preparation method of silica core material can be carried by the hydrolysis of tetraethylorthosilicate (TEOS) or tetramethyl orthosilicate (TMS) in the presence of alcohol or water solution and under basic (Stober) or acidic catalytic conditions.

Mesoporous silica particles can be prepared by hydrolysis of TEOS or TMS at low temperatures, e.g., in a temperature not exceeding 60° C., followed by dehydration by centrifugation and/or evaporation under airflow or vacuum conditions.

Dense particles can be prepared utilizing intense heating in a process called calcination. Typically, such process takes place at high temperatures at about 250° C.

In some embodiments, the core is POSS, and said POSS does not require any further functionalization; rather, the core already includes antimicrobial active units and polymerizable units attached thereto (via siloxane moieties).

In one embodiment, this invention provides an antimicrobial particle comprising POSS as the core, antimicrobial active units and polymerizable units attached to said POSS core via the siloxane units of the POSS.

In one embodiment, this invention provides a method of preparing antimicrobial POSS, comprising:
Optionally, alkylating an antimicrobial siloxane precursor with an alkylating agent, to form a quaternary ammonium, tertiary amine or any combination thereof,
Mixing the optionally alkylated antimicrobial siloxane precursor with a polymerizable siloxane precursor;
Hydrolyzing said siloxane precursors, forming the antimicrobial POSS; and
Providing work-up conditions to obtain the antimicrobial POSS particles.

In some embodiment, the antimicrobial siloxane precursor is Formula (IC), as described hereinabove. In one embodiment, the antimicrobial siloxane precursor is an alkyl-trialkoxysilane, trialkoxyarylsilane, trihaloalkylsilane ortrihaloarylsilane, wherein said alkyl or aryl is end-terminated with an anti-microbial group as described hereinabove. In another embodiment, the antimicrobial siloxane precursor is 3-aminopropyltriethoxysilane (APTES) or [3-(2-Aminoethylamino)propyl]trimethoxysilane (AEAPTS).

In some embodiments, the polymerizable precursor is an alkyl-trialkoxysilane, trialkoxyarylsilane, trihaloalkylsilane or trihaloarylsilane, wherein said alkyl or aryl is end-terminated with Z₃ of Formula (7) as described hereinabove. In one embodiment, the polymerizable precursor is 3-(Trimethoxysilyl)propyl acrylate.

In some embodiments, the alkylating agent is a C1-C20 alkyl functionalized with a leaving group selected from ethoxy, methoxy, sulfonate (e.g., mesyl, tosyl) and halide. In one embodiment the alkylating agent is iodomethane or 1-iodooctane. In another embodiment, the alkylation is done first with first alkylating agent and at a second stage with a second alkylating agent. In another embodiment, first the alkylation is performed with 1-iodooctane and then at a second stage with iodomethane.

In some embodiments, the hydrolysis is acidic or basic. In one embodiment, the hydrolysis is basic. In one embodiment, the hydrolysis is acidic. In one embodiment, the hydrolysis is basic and performed using any suitable base in the art (e.g., NaHCO₃, NaOH, Na₂CO₃). In one embodiment, the hydrolysis is acidic and performed using any suitable acid in the art (e.g., HCl, HOAc, CF₃CO₂H).

In some embodiments, the work up conditions may comprise any suitable technique as known in the art. In one embodiment, the work up comprises freeze-drying and then mechanically powdering of the particles, providing dry antimicrobial POSS particles.

In some embodiments, this invention provides an antimicrobial POSS particle prepared according to any one of the hereinabove methods.

In some embodiments, provided herein an anti-microbial particle, comprising a polyhedral oligomeric silsesquioxane (POSS) core wherein each silicon atom of the POSS is functionalized by at least one quaternary ammonium group or by at least one functional polymerizable group; and wherein the molar ratio of the silicon atom functionalized by at least one quaternary ammonium and the silicon atom functionalized at least one functional polymerizable group is between 10:1 to 1:10, respectively. In one embodiment, the POSS antimicrobial particle comprises antimicrobial active units and polymerizable units attached to said POSS core via the siloxane units of the POSS.

In other embodiments, the method for the preparation of the anti-microbial particle provided herein comprises:
mixing a first monomeric unit comprising trialkoxysilane linked to a quaternary ammonium group by a linker and a second monomeric unit comprising trialkoxysilane linked to a functional polymerizable group by a linker in a ratio of between 10:1 and 1:10, respectively; and
hydrolyzing the first and second monomeric units in basic conditions to polymerize and form a POSS core which is functionalized by an anti-microbial group and a functional polymerizable group.

In some embodiments, the particle is prepared by hydrolysis of N-alkylated [3-(2-Aminoethylamino)propyl]trimethoxysilane (AEAPTS) and 3-(Trimethoxysilyl)propyl acrylate. In other embodiments, the hydrolysis is basic. In other embodiments, the N-alkylation of AEAPTS comprises alkylation with 1-iodoctane followed by alkylation with iodomethane.

Preparation of the Compositions of this Invention

In some embodiments, the composites of this invention are prepared by embedding the anti-microbial particles into the polymeric materials of this invention. In another embodiment, one type of particle is embedded in the polymeric materials. In another embodiment, a combination of different particle types is embedded in the polymeric materials. In some embodiments, the embedding may be achieved by a variety of methodologies.

In some embodiments, embedding functionalized microparticles into a polymeric material is obtained by two methodologies: A) Extrusion technology: the particles are added into molten thermoplastic polymer into an extruder, e.g., into a twin-coned extruder. B) A thermoplastic or thermoset polymer is heated in an organic solvent (non-limiting examples comprise xylene, toluene, their derivatives or any combination thereof) under reflux conditions to achieve the complete dissolution of the polymer. The anti-microbial particles are then dispersed in the same solvent as used for the polymer and the mixture is added to the dissolved polymer using overhead stirrer or homogenizer. After complete dispersion of particles within the polymer, the solvent is evaporated using conventional distillation or evaporation method.

In some embodiments, embedding functionalized microparticles into a silicone based polymeric material is obtained by several methodologies: A) Room temperature vulcanization (RTV) of silicone precursor is achieved, wherein particles are incorporated into unpolymerized or pre-polymerized silicone before final curing at final concentration of 0.5-8% wt particles/silicone polymer. In another embodiment, the curing is activated by moisture. In another embodiment, the curing is activated by heat. B) RTV of silicone precursor is achieved, wherein polymerization is induced by mixing two components of the polymerization mixture. In another embodiment, particles are incorporated into both parts at final concentration of 0.5-8% wt. particles/silicone polymer, or in one of the parts at doubled concentration, giving the 0.5-8% wt. particles/silicone polymer final concentration.

Thus, according to some embodiments, this invention provides a method for preparing a composition comprising embedding a plurality of anti-microbial particles in a polymeric material as described above, wherein the particles are embedded in the material, the method comprises a step of adding the particles as described above, into a molten polymer material utilizing extrusion or to a polymer solution in solvent or via polymerization with the particles and polymer precursors. In one embodiment, an identical substituent (e.g., R₁, R₂, R₃ etc.) within particles found in the “plurality of particles” has the same scope as indicated above in all said particles; however, the specific value in each one of the particles for the same substituent can be different; e.g., R₁=methyl in “particle #1”, but R₁=propyl in “particle #2”, thus at least due to this and in this specific embodiment, particles 1-2 are different.

In some embodiments, particles according to this invention are homogeneously distributed on the outer surface of the polymeric material in a surface concentration of between about 0.1 to about 100 particles per sq. micrometer. In another embodiment, particles according to this invention are homogeneously distributed on the outer surface of the polymeric material in a surface concentration of between about 1 to about 100 particles per sq. micrometer. The term “homogeneous distribution” is used to denote a distribution, characterized in that the standard deviation of the number of particles per sq. um is no more than the average number of particles per sq. micrometer. A homogeneous distribution may be advantageous for reproducibility and product specifications. If the distribution is not even, the product may exhibit different properties at different areas. The distribution of the particles away from the outer surface, that is, their bulk concentration, may be similar to that on the outer surface. As a general rule, the total surface of the particles may occupy at most about 20% of the surface of the material, for example, the total surface of the particles may occupy between 1% to 15%, or, in some embodiments, between 1% and 5% or between 1% and 3% of the surface of the material.

According to some embodiments, on the average, every sq. micrometer of the outer surface of polymeric material has at least one particle of this invention.

### Compositions and Methods of Use Thereof

According to another aspect of the invention there is provided a method for inhibition of bacteria, by contacting the bacteria with an anti-microbial particle of this invention, or a composition or pharmaceutical composition comprising the particle(s) of this invention. The term “inhibition” may refer to any of the following: destruction, e.g., annihilation, of at least 99% of the bacteria, at least 99.9% of the bacteria, of at least 99.99% of the bacteria; reduction in the growth rate of the bacteria; reduction in the size of the population of the bacteria; prevention of growth of the bacteria; causing irreparable damage to the bacteria; destruction of a biofilm of such bacteria; inducing damage, short term or long term, to a part or a whole existing biofilm; preventing formation of such biofilm; inducing biofilm management; or bringing about any other type of consequence which may affect such population or biofilm and impose thereto an immediate or long term damage (partial or complete).

The term “biofilm” refers to a population of biological species (bacteria) attached to a solid surface.

In some embodiments, the inhibition is achieved by contacting the bacteria with a matrix containing up to 5% w/w, or e.g., up to 1% particles according to this invention, or compositions comprising them.

In one embodiment, this invention further provides a composition or a pharmaceutical composition comprising anti-microbial particles as referred hereinabove. In another embodiment, the composition/pharmaceutical composition comprises one type of particle. In another embodiment, the composition/pharmaceutical composition comprises a combination of different particle types. In one embodiment, non-limiting examples for a composition/pharmaceutical composition of this invention are dental adhesives, bone cement, dental restorative materials such as all types of composite based materials for filling tooth-decay cavities, endodontic filling materials (cements and fillers) for filling the root canal space in root canal treatment, materials used for provisional and final tooth restorations or tooth replacement, including but not restricted to inlays, onlays, crowns, partial dentures (fixed or removable) dental implants, and permanent and temporary cements used in dentistry for various known purposes, dental and orthopedic resin based cements, sealers, composite materials, adhesives and cements, dental restorative composites, bone cements, tooth pastes, lotions, hand-sanitizers, ointments and creams used for dermatology, wound care or in the cosmetic industry, plastic wear for medical and research laboratories; food packaging, mainly for dairy products and fresh meat and fish; pharmaceuticals packaging, paints for ships, that prevent growth of biofilm or treats, breaks down and/or kills a biofilm or bacteria within, paints for bathrooms, paint for hospitals and clean rooms; water filtration media and many others. Each possibility represents a separate embodiment of this invention. In some embodiments, the particles or composition comprising thereof are used for dental and orthopedic resin based cements, sealers, composite materials, adhesives and cements; for dental and orthopedic metal implants and wires; for surgical sutures; for catheters, metal surgical tools, non-surgical medical devices. Each possibility represents a separate embodiment of this invention.

In one embodiment the composition or composite of this invention is a varnish or glaze which is applied to the tooth surface, a restoration of tooth or a crown comprising the particles of this invention. In another embodiment the varnish or glaze provide a protective coating, lacquer; superficially polished appearance to the tooth surface, restoration or crown of the tooth. In another embodiment, the varnish is a fluoride varnish which is a highly concentrated form of fluoride which is applied to the tooth's surface, as a type of topical fluoride therapy. In another embodiment, the aim of glazing is to seal the open pores in the surface of a fired porcelain. Dental glazes are composed of colorless glass powder, applied to the fired crown surface, so as to produce a glossy surface. Unglazed or trimmed porcelain may also lead to inflammation of the soft tissues it contacts.

In one embodiment, the composition/pharmaceutical composition of this invention is in a form selected from the group consisting of a cream, an ointment, a paste, a dressing and a gel, for example, wherein the composition is formulated for topical application or administration. In another embodiment, the composition is intended for administration into an oral cavity. The composition may be formulated as a tooth paste, and/or may be applied to a surface or medical device selected from the group consisting of a denture cleaner, post hygienic treatment dressing or gel, mucosal adhesive paste, a dental adhesive, a dental restorative composite based material for filling tooth, decay cavities, a dental restorative endodontic filling material for filling root canal space in root canal treatment, a dental restorative material used for provisional and final tooth restorations or tooth replacement, a dental inlay, a dental onlay, a crown, a partial denture, a complete denture, a dental implant and a dental implant abutment.

In one embodiment, the pharmaceutical composition further comprises at least one pharmaceutically active ingredient. In another embodiment, non-limiting examples of pharmaceutically active ingredients include Analgesics, Antibiotics, Anticoagulants, Antidepressants, Anticancers, Antiepileptics, Antipsychotics, Antivirals, Sedatives and Antidiabetics. In another embodiment, non-limiting examples of Analgesics include paracetamol, non-steroidal anti-inflammatory drugs (NSAIDs), morphine and oxycodone. In another embodiment, non-limiting examples of Antibiotics include penicillin, cephalosporin, ciprofolxacin and erythromycin. In another embodiment, non-limiting examples of Anticoagulants include warfarin, dabigatran, apixaban and rivaroxaban. In another embodiment, non-limiting examples of Antidepressants include sertraline, fluoxetine, citalopram and paroxetine. In another embodiment, non-limiting examples of Anticancers include Capecitabine, Mitomycin, Etoposide and Pembrolizumab. In another embodiment, non-limiting examples of Antiepileptics include Acetazolamide, Clobazam, Ethosuximide and lacosamide. In another embodiment, non-limiting examples of Antipsychotics include Risperidone, Ziprasidone, Paliperidone and Lurasidone. In another embodiment, non-limiting examples of Antivirals include amantadine, rimantadine, oseltamivir and zanamivir. In another embodiment, non-limiting examples of Sedatives include Alprazolam, Clorazepate, Diazepam and Estazolam. In another embodiment, non-limiting examples of Antidiabetics include glimepiride, gliclazide, glyburide and glipizide.

In another embodiment, the pharmaceutical composition further comprises excipients.

In another embodiment, the excipient comprises binders, coatings, lubricants, flavors, preservatives, sweeteners, vehicles and disintegrants. In another embodiment, non-limiting examples of binders include saccharides, gelatin, polyvinylpyrolidone (PVP) and polyethylene glycol (PEG). In another embodiment, non-limiting examples of coatings include hydroxypropylmethylcellulose, polysaccharides and gelatin. In another embodiment, non-limiting examples of lubricants include talc, stearin, silica and magnesium stearate. In another embodiment, non-limiting examples of disintegrants include crosslinked polyvinylpyrolidone, crosslinked sodium carboxymethyl cellulose (croscarmellose sodium) and modified starch sodium starch glycolate.

In one embodiment, the invention is directed to a packaging composition comprising a thermoplastic polymer and/or hydrogel embedded with anti-microbial particles as referred hereinabove. In another embodiment, the thermoplastic polymer and/or hydrogel is embedded with a mixture of two or more different particles. In another embodiment, the packaging composition is used in the packaging of food, beverage, pharmaceutical ingredients, medical devices, surgical equipment before operation, pre operation equipment, cosmetics, and sterilized equipment/materials.

In one embodiment the packaging composition comprises a thermoplastic polymer and/or hydrogel embedded with the particles as referred hereinabove. In another embodiment, the thermoplastic polymer is polyvinylchloride (PVC), polyethylene, polypropylene, silicone, epoxy resin or acrylic polymers. In another embodiment, the thermoplastic polymer is poly methylmethacrylate or polyurethane.

In another embodiment, the packaging composition further comprises binders, coatings, lubricants and disintegrants. In another embodiment, non-limiting examples of binders include saccharides, gelatin, polyvinylpyrolidone (PVP) and polyethylene glycol (PEG). In another embodiment, non-limiting examples of coatings include hydroxypropylmethylcellulose, polysaccharides and gelatin. In another embodiment, non-limiting examples of lubricants include talc, stearin, silica and magnesium stearate. In another embodiment, non-limiting examples of disintegrants include crosslinked polyvinylpyrolidone, crosslinked sodium carboxymethyl cellulose (croscarmellose sodium) and modified starch sodium starch glycolate.

In one embodiment, the packaging composition is used for packaging pharmaceutical ingredients. In another embodiment, non-limiting examples of pharmaceutical ingredients include analgesics, antibiotics, anticoagulants, antidepressants, anti-cancers, antiepileptics, antipsychotics, antivirals, Sedatives and antidiabetics. In another embodiment, non-limiting examples of analgesics include paracetamol, non-steroidal anti-inflammatory drugs (NSAIDs), morphine and oxycodone. In another embodiment, non-limiting examples of antibiotics include penicillin, cephalosporin, ciprofloxacin and erythromycin. In another embodiment, non-limiting examples of anticoagulants include warfarin, dabigatran, apixaban and rivaroxaban. In another embodiment, non-limiting examples of Antidepressants include sertraline, fluoxetine, citalopram and paroxetine. In another embodiment, non-limiting examples of anti-cancers include Capecitabine, Mitomycin, Etoposide and Pembrolizumab. In another embodiment, non-limiting examples of antiepileptics include Acetazolamide, Clobazam, Ethosuximide and lacosamide. In another embodiment, non-limiting examples of antipsychotics include Risperidone, Ziprasidone, Paliperidone and Lurasidone. In another embodiment, non-limiting examples of antivirals include amantadine, rimantadine, oseltamivir and zanamivir. In another embodiment, non-limiting examples of sedatives include Alprazolam, Clorazepate, Diazepam and Estazolam. In another embodiment, non-limiting examples of antidiabetics include glimepiride, gliclazide, glyburide and glipizide.

In one embodiment, the packaging composition is used in the packaging of food ingredients. In another embodiment, non-limiting examples of food ingredients packaged with the packaging material of the invention include fresh food, preservatives, sweeteners, color additives, flavors and spices, nutrients, emulsifiers, binders and thickeners. In another embodiment, non-limiting examples of fresh food include: meat, poultry, fish, dairy products, fruits and vegetables. In another embodiment, non-limiting examples of preservatives include Ascorbic acid, citric acid, sodium benzoate, calcium propionate, sodium erythorbate, butylated hydroxy toluene (BHT), silver, chlorhexidine, trichlozan and sodium nitrite. In another embodiment, non-limiting examples of sweeteners include Sucrose (sugar), glucose, fructose, sorbitol, mannitol and corn syrup. In another embodiment, non-limiting examples of color additives include Orange B, Citrus Red No. 2, annatto extract, beta-carotene, grape skin extract, cochineal extract or carmine and paprika oleoresin. In another embodiment, non-limiting examples of flavors and spices include monosodium glutamate, glycine slats, inosinic acid, isoamyl acetate, and limonene and allyl hexanoate. In another embodiment, non-limiting examples of nutrients include Thiamine hydrochloride, riboflavin (Vitamin B₂), niacin, niacinamide, folate or folic acid. In another embodiment, non-limiting examples of emulsifiers include Soy lecithin, mono- and diglycerides, egg yolks, polysorbates and sorbitan monostearate. In another embodiment, non-limiting examples of binders and thickeners include Gelatin, pectin, guar gum, carrageenan, xanthan gum and whey.

In one embodiment, this invention provides a method for inhibiting or preventing biofilm formation, comprising applying onto a susceptible or infected surface or a medical device a composition of this invention.

In another embodiment, this invention provides a composition of this invention for use in inhibiting or preventing a biofilm formation.

In one embodiment, this invention provides a method for inhibiting or preventing biofilm formation or growth comprising placing a medical device of this invention (comprising a composition of this invention as referred hereinabove) on the surface to be treated. In another embodiment, the medical device is a wound dressing.

In another embodiment, this invention provides a medical device of this invention for use in inhibiting or preventing biofilm formation or growth.

In one embodiment, this invention provides a method for inhibition of bacteria, the method comprising the step of contacting the bacteria with the pharmaceutical or packaging composition or composite of this invention.

In another embodiment, this invention provides a pharmaceutical or packaging composition or for use in inhibiting bacteria.

In one embodiment, this invention provides a method for treating, breaking down or killing biofilm or bacteria within, comprising applying onto a susceptible or infected surface or a medical device the pharmaceutical or packaging composition or composite of this invention.

In another embodiment, this invention provides a composite or a pharmaceutical or packaging composition of this invention for use in treating, breaking down or killing biofilm or bacteria within.

Applications out of the medical field may for example be in clothing (e.g., for sports or outdoor activity; to prevent bacteria-induced sweat odor), athlete shoes or the inner part of a shoe wherein bacteria tend to collect, sportswear and clothing for outdoor activity, tooth brushes and any brush that are in contact with the human body, air and water filters, water treatment and distribution systems, pet cages as well as other veterinary items, etc.

In some embodiments, the anti-microbial compositions or composites of this invention affect annihilation of at least about 99% of the contacted bacteria, or in some embodiments at least about 99.9% or 99.99% of the contacted bacteria.

It was further surprisingly discovered that the particles within compositions/composites/medical devices of this invention maintain high anti-microbial properties over time without leaching out and with no alteration of the properties of the hosting matrix. Such particles demonstrate enhanced anti-bacterial activity originating from the presence of closely packed anti-bacterial groups on a given particle's surface.

Medical Devices of this Invention

In one embodiment, this invention further provides a medical device comprising a composition of this invention. In one embodiment, non-limiting examples for medical devices of this invention are catheters, stents, surgical mesh, breast implants, joint replacements, artificial bones, artificial blood vessels, artificial heart valves (cardiology), artificial skin, plastic surgery implants or prostheses, intra uterine devices (gynecology), neurosurgical shunts, contact lenses (ophthalmology), intraocular lenses, ocular prosthesis, urethral stents, coating for subcutaneous (such as orthopedic or dental) implants, insulin pumps, contraceptives, pacemakers, tubing and cannulas used for intra venous infusion, tubing and cannulas used for dialysis, surgical drainage tubing, urinary catheters, endotracheal tubes, wound covering (dressing and adhesive bandage) and treatment (e.g., gels, ointments, pastes and creams for wound care which reduce biofilm and bacteria to aid wound healing) materials, sutures, catheters of all kinds that are inserted temporarily or permanently in blood vessels as well as the urinary system, shunt for use in brain applications, surgical gloves, tips for ear examination, statoscope ends and other elements used by the medical personnel; tooth brushes, tooth pick, dental floss, interdental and tongue brushes, surgical sutures, metal surgical tools, non-surgical medical devices, dental, and orthopedic metal implants and wires and surgical drains, syringes, trays, tips, gloves and other accessories used in common medical and dental procedures.

In one embodiment, this invention further provides a medical device comprising a dental appliance. In one embodiment, this invention further provides a medical device comprising an orthodontic appliance. The dental appliance and the orthodontal appliance comprise the particles and composition of this invention. In some embodiments, the orthodontal appliance include an aligner for accelerating the tooth aligning, a bracket, a dental attachment, a bracket auxiliary, a ligature tie, a pin, a bracket slot cap, a wire, a screw, a micro-staple, cements for bracket and attachments and other orthodontic appliances, a denture, a partial denture, a dental implant, a periodontal probe, a periodontal chip, a film, or a space between teeth. In some embodiments, the dental appliance may include a mouth guard, used to prevent tooth grinding (bruxer, Bruxism), night guard, an oral device used for treatment/prevention sleep apnea, teeth guard used in sport activities.

In one embodiment, this invention further provides a trans dermal medical device such as orthopedic external fixation screws and wires used for bone fixations and stabilization and trans mucosal elements used in dental implants such as healing caps, abutments (such as multiunit), for screw retained or for cement retained dental prosthesis.

In one embodiment, this invention further provides a medical device comprising an endoscope (rigid and flexible), including, and not limited to a colonoscope, gastroscope, duodenoscope, bronchoscope, cystoscope, ENT scopes, laparoscope, laryngoscope and similar instruments for examination or treatment the inside of the patient's body, including any parts thereof, as well as accessories and other devices used in the procedure which either come in contact with body tissue or fluids; tubes, pumps, containers and connectors (used inside or outside the body) through which fluids, air or gas may be pumped into or suctioned out from the patient and could become contaminated by the patient or transfer contaminants from other patients; items such as brushes, trays, covers, tubes, connectors cabinets and bags used for reprocessing, cleaning, transporting and storing such equipment and can transmit or host biological contaminants, as well as filters for air or water used in dental or medical procedures, hospital surfaces (such as floors, tabletops), drapes, curtains, linen, handles and the like.

The antimicrobial property may protect the patient and the medical staff from cross contamination from patient to patient or from patient to the examiner. Self-sterilizing packaging for medicines and items that enter the operation room are also beneficial.

In one embodiment, this invention further provides processes for preparing the medical devices comprising the composites. In another embodiment, the medical devices are prepared via the steps of: providing a fluid phase of the composite of this invention; shaping the fluid; and hardening of the shaped fluid, affording the desired medical device. In another embodiment, the medical devices are prepared via the steps of: providing a solid phase of the composite; and shaping of the solid, affording the desired medical device. In another embodiment, the shaping is accomplished via extrusion or molding. In another embodiment, fluid phase of the composite comprises melted composite or a composite dissolved in a solvent.

Another polymer material to be used in the context of this invention is resins used in dental, surgical, chirurgical and orthopedic composite materials. In such applications, anti-microbial particles could be first dispersed within the resin part or added simultaneously with filler or any other solid ingredients (if any). Most of these resins are acrylic or epoxy type monomers that undergo polymerization in-vivo.

In some embodiments, the terms “anti-microbial” and “anti-bacterial” are used herein interchangeably.

The following examples are presented in order to more fully illustrate the preferred embodiments of this invention. They should in no way, however, be construed as limiting the broad scope of this invention.

### EXAMPLES

### Example 1

### Preparation of QASi-PF Particles (No Acrylate)

[3-(2-Aminoethylamino)propyl]trimethoxysilane (AEAPTS) (1 eq.; 10% w/w in THF) was reacted with 1-iodooctane (2 eq.) and then with iodomethane (4 eq.) to convert all amines into hydrophobic quaternary ammonium groups. Subsequently, silane groups were hydrolyzed in aqueous NaHCO₃ solution followed by condensation and POSS particles formation.

Dry POSS particles were obtained by freeze-drying and then mechanically powdering.

### Example 2

Preparation of QASi-PA Particles (with Acrylate)

[3-(2-Aminoethylamino)propyl]trimethoxysilane (AEAPTS) (1 eq.; 10% w/w in THF) was reacted with 1-iodooctane (2 eq.) and then with iodomethane (4 eq.) to convert all amines into hydrophobic quaternary ammonium groups. 3-(Trimethoxysilyl)propyl acrylate (0.25 eq.) were added to the solution. Subsequently, silane groups were hydrolyzed in aqueous NaHCO₃ solution followed by condensation and POSS particles formation. Dry POSS particles were obtained by freeze-drying and then mechanically powdering.

### Example 3

### Preparation of Dental Composites

### Resin Preparation:

Bisphenol A glycidyl ether dimethacrylate (BisGMA), triethyleneglycol dimethacrylate (TEGDMA), urethane dimethacrylate (UDMA) were mixed together until clear blend. 0.10% of camphorquinone (CQ), 0.1% of benzoin methyl ether (BME), 0.10% of ethyl 4-(dimethylamino) benzoate (EDB) and 0.01% of butylated hydroxytoluene (BHT) were added and mixed until complete dissolution.

### Composite Kneading:

Into prepared resin, glass powder was added to arrive at ratio of 78.5% filler and 20% of resin; and 1.5% of QASi particles (Example 1 or Example 2). Composites were mixed under vacuum using planetary kneading machine until homogeneous paste.

### Example 4

### Leaching Tests of the Particles—Validation

The following validates a limit test to detect QASi on polar and non-polar extracts. During the study the Specificity and the Limit of detection were verified, using the sample preparation and biological evaluation guidelines of ISO 10993-12 and 18. A HPLC/MS (high performance liquid chromatography mass spectrometry) system was used (Agilent 1260 HPLC coupled with a LC/MS Triple Quad), with which the analytical method for the analyte quantification was developed, and with standard laboratory equipment and reagents (water, acetonitrile, formic acid, ethanol and NaCl 0.9%).

MS1 standard solution 500 g/ml of QASi-PF was prepared using about 12.5 mg of the particles dissolved and diluted to 25 ml with ethanol; and diluted as illustrated in the following table.

| | Starting | Starting | | Final | |
|---|---|---|---|---|---|
| Concentration | solution | volume | | volume | Solvent |
| 20 μg/ml | MS1 | 0.2 | ml | 5 ml | Ethanol:Water |
| (MS3) | | | | | 50:50 |
| 10 μg/ml | MS3 | 0.5 | ml | 1 ml | |
| 1 μg/ml | | 0.05 | ml | 1 ml | |

MS2 standard solution 500 g/ml of QASi-PA was prepared using about 12.5 mg of the particles dissolved and diluted to 25 ml with ethanol; and diluted as illustrated in the following table.

| | Starting | Starting | | Final | |
|---|---|---|---|---|---|
| Concentration | solution | volume | | volume | Solvent |
| 20 μg/ml | MS2 | 0.2 | ml | 5 ml | Ethanol:Water |
| (MS4) | | | | | 50:50 |
| 10 μg/ml | MS4 | 0.5 | ml | 1 ml | |
| 1 μg/ml | | 0.05 | ml | 1 ml | |

A preliminary scan in ESI+ mode from m/z 100 to m/z 1000 was performed in order to find the precursor ion for each reference. The ion signal 266.3 m/z was found for QASi-PF and the ion signal 273.2 m/z was found for QASi-PA. The following conditions were used to

| | |
|---|---|
| Parameter | Settings |

| | |
|---|---|
| Column | Phenomenex Luna 4.6 mm × |
| | 150 mm × 5 μm (C1041 HP) |
| Column temperature | 60° C. |
| Mobile phase | A) 0.1% Formic Acid in water |
| | B) Acetonitrile |

| Time (min) | % A | % B |
|---|---|---|
| 0.01 | 95 | 5 |
| 10.00 | 0 | 100 |
| 14.00 | 0 | 100 |
| 14.50 | 95 | 5 |
| 18.00 | 95 | 5 |

| | |
|---|---|
| Flow rate | 0.5 ml/min |
| Detection | MS ESI+ |
| Injection volume | 1 μl positive mode |
| Masses | 266.3-273.2 |

test the samples, and under these conditions 1 g/ml samples were detected in both solvents and without interfering signals.

Accordingly, it was concluded that the method is able to assess the presence of masses at 266.3 and 273.2, which are not present on the solvent blanks, in order to verify the presence of QASi leaching from an extracted device.

### Example 5

### Leaching Test of QASi-PF and QASi-PA Particles (Examples 1 and 2, Respectively) in a Dental Composite (a Composite Material for Cavity Filling and Tooth Restoration)

The particles were prepared as in Examples 1 and 2, compositions were prepared as in Example 3 and the leachability test was performed as in Example 4. The following solutions and concentrations were used.

| Concentrati [symbol] | | Starti [symbol] | Startin [symbol] | | Final [symbol] | Solven [symbol] |
|---|---|---|---|---|---|---|
| 2 | μg/ml | MS2 | 0.1 | ml | 1 ml | Ethanol:Water |
| 5 | μg/ml | | 0.25 | ml | 1 ml | 50:50 |
| 10 | μg/ml | | 0.5 | ml | 1 ml | |
| 2 | μg/ml | MS3 | 0.1 | ml | 1 ml | NaCl 0.9% |
| 5 | μg/ml | | 0.25 | ml | 1 ml | |
| 10 | μg/ml | | 0.5 | ml | 1 ml | |

[symbol]

indicates data missing or illegible when filed

The low leachability of QASi-PA particles (with acrylate) was demonstrated in comparison to QASi-PF particles (no acrylate), which were more easily removed from the matrix, as exemplified in the following.

The results for QASi-PA particles (with acrylate) for both types of solutions are the following, with the conclusion that the extraction of the QASi-PA has to be considered exhaustive at 120 hours in Ethanol:Water and 96 hours in saline solution. A total of 268.2 pg/g of QASi-PA were leached by Ethanol:Water while no QASi-PA was leached by saline solution at exhaustive conditions. Hence, QASi-PA particles (with acrylate) were shown to have low leachability from the matrix.

### QASi-PA Results:

EtOH:Water (50:50)

| Time | 72 | 96 hours | 120 hours | Total |
|---|---|---|---|---|
| Total QASi | 224.7 | 25.0 | 18.5 | 268.2 |
| % of T1 | // | 11.1% | 8.2% | |
| | | [symbol] | [symbol] | |

[symbol]

indicates data missing or illegible when filed

NaCl 0.9%

| Time | 72 | 96 hours | Total |
|---|---|---|---|
| Total QASi | <10 | <10 | <10 |
| % of T1 | // | // | |
| | | [symbol] | |

[symbol]

indicates data missing or illegible when filed

### QASi-PF Results (Control):

The extraction of the QASi-PF has to be considered exhaustive at 96 hours for both extraction solvents. A total of 1454.4 μg/g of QASi-PF were leached by Ethanol:Water while no QASi-PF was leached by saline solution at exhaustive conditions.

EtOH:Water (50:50)

| Time | 72 | 96 hours | Total |
|---|---|---|---|
| Total QASi | 1363.5 | 90.9 | 1454.4 |
| % of T1 | // | 6.7% | |
| | | [symbol] | |

[symbol]

indicates data missing or illegible when filed

NaCl 0.9%

| Time | 72 | 96 hours | Total |
|---|---|---|---|
| Total QASi | <10 | <10 | <10 |
| % of T1 | // | // | |
| | | [symbol] | |

[symbol]

indicates data missing or illegible when filed

Accordingly, leaching in particles absent of acrylate (≥1400 mg/g) was far more significant than the corresponding value in particles comprising acrylate (268.2 mg/g).

### Example 6

### Leaching Test of QASi-PA Particles (Example 2) in Bond (a Dental Adhesive for Fixing Direct and Indirect Tooth Restorations)

A leachability test as provided in Example 4 was performed to detect QASi on the test item after exhaustive extraction with NaCl 0.9% and Water:Ethanol 50:50 solution following ISO10993-12 guidelines. Discs of the bonded material were completely soaked in the extraction solvent, using a solvent ratio of 0.2 g/ml. Two solvents were used: NaCl 0.9% and Water/Ethanol (50/50), and the samples were kept at the following conditions: (i) 72±2 hours at 50±2° C., (ii) 96±2 hours at 50±2° C., and (iii) 120±2 hours at 50±2° C. (tested only in case exhaustivity was not reached at 96 hours). Amounts of each mixture were left at the same condition of the samples to be used as blank. The solutions which were used are: MS1 standard solution 500 μg/ml that included about 12.5 mg of the QASi-PA dissolved and diluted to 25 ml with ethanol; MS2 standard solution 20 μg/ml (Ethanol:Water 50:50) that included 0.2 ml of MS1 diluted to 5 ml with Ethanol:Water 50:50; and MS2 standard solution 20 μg/ml (NaCl 0.9%) that included 0.2 ml of MS1 diluted to 5 ml with NaCl 0.9%.

| | | Starting | Starting | | Final | |
|---|---|---|---|---|---|---|
| Concentration | | solution | volume | | volume | Solvent |
| 2 | μg/ml | MS2 | 0.1 | ml | 1 ml | Ethanol:Water |
| 5 | μg/ml | | 0.25 | ml | 1 ml | 50:50 |
| 10 | μg/ml | | 0.5 | ml | 1 ml | |
| 2 | μg/ml | MS3 | 0.1 | ml | 1 ml | NaCl 0.9% |
| 5 | μg/ml | | 0.25 | ml | 1 ml | |
| 10 | μg/ml | | 0.5 | ml | 1 ml | |

Sample Preparations—The samples were injected without any further treatment and after dilution when needed. For 1:100 Dilution, 0.1 ml of each of the extraction solutions was diluted to 10 ml with the same extraction solvent. For 1:10 Dilution, 0.1 ml of each of the extraction solutions was diluted to 1 ml with the same extraction solvent. Leachability testing setting was as in Example 4.

The results are shown in the tables below. The extraction of the QASi-PA has to be considered exhaustive at 96 hours in Ethanol:Water and 120 hours in saline solution.

A total of 436.5 μg/g of QASi-PA were leached by Ethanol:Water and 66.8 μg/g QASi-PA was leached by saline solution at exhaustive conditions.

EtOH:Water (50:50)

| Time | 72 | 96 hours | Total |
|---|---|---|---|
| Total QASi | 413.2 | 23.3 | 436.5 |
| % of T1 | // | 5.6% | |
| | | [symbol] | |

[symbol]

indicates data missing or illegible when filed

NaCl 0.9%

| Time | 72 | 96 hours | 120 | Total |
|---|---|---|---|---|
| Total QASi | 55.2 | 11.6 | <10 | 66.8 |
| % of T1 | // | 21.0% | // | |
| | | [symbol] | [symbol] | |

[symbol]

indicates data missing or illegible when filed

### Example 7

Preparation of QASi-PV Particles (with Silicone Rubber)

[3-(2-Aminoethylamino)propyl]trimethoxysilane (AEAPTS) (1 eq.; 10% w/w in THF) was reacted with 1-iodooctane (2 eq.) and then with iodomethane (4 eq.) to convert all amines into hydrophobic quaternary ammonium groups. Vinyltrimethoxysilane (0.25 eq.) were added to the solution. Subsequently, silane groups were hydrolyzed in aqueous NaHCO₃ solution followed by condensation and POSS particles formation. Dry POSS particles were obtained by freeze-drying and then mechanically powdering.

### Preparation of Silicone Rubber

Composite kneading: Pt-catalyst silicone rubber was prepared by mixing unpolymerized silicone with 2% wt/wt QASi-PV and then cured at 80° C. for 2 hours. Control samples with 2% wt/wt QASi-PF were prepared simultaneously in the same conditions.

### Leachability Study

10 gr samples of each silicone (test and control) were immersed in 25 ml of saline and kept at 37° C. for 72 hours. The liquid then was collected and examined using UV spectrophotometer. To determine the UV absorbance intensity correlation to the QASi concentration in the liquid, calibration curve of standard concentrations in saline were prepared and tested using the same UV spectrophotometer.

The results were that the QASi-PV concentration in saline after 72 hours: 0 mg per 10 gr of silicone sample—indicating that the particles remain in the silicone matrix. This in contrast to QASi-PF (control) concentration in saline after 72 hours: 25 mg per 10 gr of silicone sample—indicating the leaching of the control particles (without the vinyl groups).

### Example 8

Preparation of QASi-PE Particles (with Epoxy)

[3-(2-Aminoethylamino)propyl]trimethoxysilane (AEAPTS) (1 eq.; 10% w/w in THF) was reacted with 1-iodooctane (2 eq.) and then with iodomethane (4 eq.) to convert all amines into hydrophobic quaternary ammonium groups. (3-Glycidyloxypropyl)trimethoxysilane (0.25 eq.) were added to the solution. Subsequently, silane groups were hydrolyzed in aqueous NaHCO₃ solution followed by condensation and POSS particles formation. Dry POSS particles were obtained by freeze-drying and then mechanically powdering.

### Preparation of Epoxy Resin Samples

Composite kneading: Diglycidyl ether of bisphenol-a (DGEBA) resin was mixed with diethylenediamine crossliner (DETA) at 5:1 ration and QASi-PE particles were added to adjust total 2% wt/wt and mixed using overhead stirrer. Samples were allowed to polymerize for 24 h at 37° C.

Control samples with 2% wt/wt QASi-PF were prepared simultaneously in the same conditions.

### Leachability Study

12 gr samples of each silicone (test and control) were immersed in 25 ml of saline and kept at 37° C. for 72 hours. The liquid then was collected and examined using UV spectrophotometer. To determine the UV absorbance intensity correlation to the QASi concentration in the liquid, calibration curve of standard concentrations in saline were prepared and tested using the same UV spectrophotometer.

The results were that the QASi-PE concentration in saline after 72 hours was 25 mg per 12 gr of silicone sample, indicating that most of the particles remain in the silicone matrix, in contrast to the QASi-PF (control) concentration in saline after 72 hours: 250 mg per 12 gr of silicone sample—indicating the leaching of the control particles (without the epoxy groups).

While certain features of this invention have been illustrated and described herein, many modifications, substitutions, changes, and equivalents will now occur to those of ordinary skill in the art. It is, therefore, to be understood that the appended claims are intended to cover all such modifications and changes as fall within the true spirit of this invention.

1. An anti-microbial particle comprising a polyhedral oligomeric silsesquioxane (POSS) core wherein the silicon atoms of the POSS are functionalized by at least one anti-microbial group and by at least one functional polymerizable group,
wherein the at least one functional polymerizable group comprises an acrylate, an epoxy, a vinyl or isocyanate group;
wherein the molar ratio of the silicon atoms functionalized by the at least one anti-microbial group and the silicon atoms functionalized by the at least one functional polymerizable group is between 10:1 to 1:10, respectively.

2. The anti-microbial particle of claim 1, wherein the at least one antimicrobial group comprises a quaternary ammonium, a tertiary ammonium or a tertiary amine.

3. (canceled)

4. (canceled)

5. The anti-microbial particle of claim 1, wherein the acrylate is propyl acrylate, wherein the propyl end group is attached to the silicon chain.

6. The anti-microbial particle of claim 1, wherein said molar ratio is between 4:1 to 1:1.

7. (canceled)

8. (canceled)

9. (canceled)

10. The anti-microbial particle of claim 1, wherein the anti-microbial particle is prepared by hydrolysis of N-alkylated [3-(2-Aminoethylamino)propyl]trimethoxysilane (AEAPTS) and 3-(Trimethoxysilyl)propyl acrylate.

11. (canceled)

12. (canceled)

13. The anti-microbial particle of claim 1, wherein the at least one functional polymerizable group comprises an epoxy group.

14. The anti-microbial particle of claim 13, wherein the epoxy group is a substituted or non-substituted 3-Glycidyloxypropyl.

15. The anti-microbial particle of claim 1, wherein the at least one functional polymerizable group comprises a vinyl group.

16. The anti-microbial particle of claim 15, wherein the vinyl group is a substituted or non-substituted vinyltrimethoxysilane.

17. (canceled)

18. (canceled)

19. A composition comprising a plurality of anti-microbial particles of claim 1 and a polymeric material as a matrix.

20. The composition of claim 19, wherein the anti-microbial particles are homogeneously dispersed in the polymeric material.

21. (canceled)

22. The composition of claim 19, wherein the polymeric material comprises organic polymer that comprises hydrogels, polyolefins, epoxy resin, acrylate resin, or any combination thereof.

23. (canceled)

24. (canceled)

25. The composition of claim 19, wherein the polymeric material comprises inorganic polymer that comprises silicone polymers ceramics, metals or any combination thereof.

26. (canceled)

27. The composition of claim 19, wherein the at least one functional polymerizable group comprises a vinyl.

28. (canceled)

29. The composition of claim 19, wherein the weight ratio of the anti-microbial particles to the polymeric material is between 0.25-5%.

30. (canceled)

31. The composition of claim 19, wherein up to 70% of the silicon atoms of the POSS are capped.

32. (canceled)

33. The composition of claim 19, wherein said composition is a dental restorative endodontic filling material for filling root canal space in root canal treatment.

34. (canceled)

35. (canceled)

36. The composition of claim 19, wherein the matrix comprises resin that is prepared by mixing Bisphenol A glycidyl ether dimethacrylate (BisGMA), triethyleneglycol dimethacrylate (TEGDMA) and urethane dimethacrylate (UDMA), providing a clear blend; followed by mixing in camphoquinone (CQ), benzoin methyl ether (BME), ethyl 4-(dimethylamino) benzoate (EDB) and butylated hydroxytoluene (BHT), providing the resin.

37. An anti-microbial particle comprising a polyhedral oligomeric silsesquioxane (POSS) core wherein the silicon atoms of the POSS are functionalized by at least one anti-microbial group and by at least one functional polymerizable group,
wherein the at least one functional polymerizable group comprises a substituted and/or non-substituted acrylate group, a substituted and/or non-substituted epoxy group, a substituted and/or non-substituted vinyl group and/or an isocyanate group; and
wherein the anti-microbial active unit is represented by structure (1): 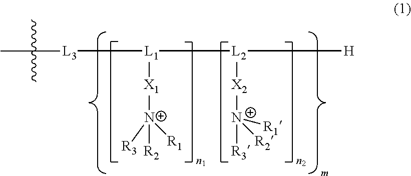
wherein
L₁ is a first linker or a bond;
L₂ is a second linker;
L₃ is a third linker or a bond;
R₁ and R₁′ are each independently alkyl, terpenoid, cycloalkyl, aryl, heterocycle, alkenyl, alkynyl or any combination thereof,
R₂ and R₂′ are each independently alkyl, terpenoid, cycloalkyl, aryl, heterocycle, alkenyl, alkynyl or any combination thereof,
R₃ and R₃′ are each independently nothing, hydrogen, alkyl, terpenoid moiety, cycloalkyl, aryl, heterocycle, a conjugated alkyl, alkenyl, alkynyl or any combination thereof; wherein if R₃ or R₃′ are nothing, the nitrogen is not charged;
X₁ and X₂ is each independently a bond, alkylene, alkenylene, or alkynylene;
n₁ is each independently an integer between 0 to 200;
n₂ is each independently an integer between 0 to 200;
wherein n₁+n₂≥1;
m is an integer between 1 to 200 and the repeating unit is the same or different; and
the notation “ [symbol] ” denotes a covalent bond to an organic or inorganic core.
In another embodiment, the anti-microbial group is —N(R₁)(R₂)(R₃)⁺, —N(R₁)(R₂)⁺—, —N(R₁′)(R₂′)(R₃′)⁺ or —N(R₁′)(R₂′)⁺— (all possibilities are connected covalently to X₁ or X₂).

38. A composition comprising a plurality of anti-microbial particles of claim 19 and a polymeric material as a matrix.

39. The composition of claim 19 wherein said composition is a dental restorative material configured for use in at least one of: provisional and final tooth restorations or tooth re lacement a dental inlay, a dental onlay, a crown, a partial denture, a complete denture, a dental implant, a dental implant abutment and a cement for permanently cementing crowns bridges, onlays, partial dentures and orthodontic appliances onto tooth enamel and dentin.
